(19) United States
(12) Patent Application Publication
(10) Pub. No.: US 2024/0059668 A1
(43) Pub. Date: Feb. 22, 2024

(54) THIOHYDROXYPYRIDINE CHELATES

(71) Applicant: LUMIPHORE, INC., Berkeley, CA (US)

(72) Inventors: Darren MAGDA, San Leandro, CA (US); David TATUM, Berkeley, CA (US)

(21) Appl. No.: 18/250,096

(22) PCT Filed: Oct. 21, 2021
(86) PCT No.: PCT/US2021/056117

§ 371 Date: Apr. 21, 2023

Related U.S. Application Data
(60) Provisional application No. 63/094,752, filed on Oct. 21, 2020.

(51) Int. Cl. C07D 401/12; C07D 498/22; C07F 3/02; C07F 3/06; C07F 1/08; C07D 401/14; C07D 471/22

(52) U.S. Cl. CPC C07D 401/12; C07D 498/22; C07F 3/02; C07F 3/06; C07F 1/08; C07D 401/14; C07D 471/22

(57) ABSTRACT

There is a provided metal ion chelating agents capable of stably binding soft Lewis acid metal ions, metal ion chelates based on these chelating agents, and methods of using the compounds of the invention in diagnostic and therapeutics methods. Also provided are methods of sequestering soft Lewis acid metal ions. Exemplary soft Lewis acid metal ions are radionuclides.

### FIELD OF THE INVENTION

The invention relates to chemical compounds and complexes that can be used in therapeutic and diagnostic applications.

### BACKGROUND OF THE INVENTION

Targeted radionuclide therapy is of growing interest and utility, and access to a wider range of radionuclides is highly desirable. Therapeutic and diagnostic agents based on soft Lewis acid ions, such as radioisotopes of copper(II) (e.g., copper-64 and copper-67), lead(II) (e.g., lead-212), gallium(III) (e.g., gallium-68), or harder Lewis acid ions, such as actinium(III), cobalt(II), manganese(III), iron(III), indium(III) and lanthanide(III) ions, are underdeveloped, though radiopharmaceutical applications such as targeted cancer imaging and therapy using copper-64 and lead-212, respectively, have been reported. The paucity of agents based on soft Lewis acid metal ions is partially attributible to a lack of bifunctional, targetable chelators capable of stable chelation of the soft Lewis acid metal ions.

For the targeting moiety on a targeted metal chelate to retain its desired target specificity, it is often undesirable to subject the chelating agent (conjugated to the targeting moiety) to extreme conditions (e.g., heat, pH) in order to complex the metal ion with the chelating agent. Current highly stable chelating agents for soft Lewis acid metal ions often require heating above ambient temperature and, for some, for prolonged periods of time to achieve acceptable complexation of the metal ion by the chelating agent. This is clearly incompatible with the goal of retaining the targeting efficacy of the targeting moiety. Thus, it remains a goal to identify classes of chelating agents that will complex soft Lewis acid metal ions at reasonably low temperatures, which do not degrade the targeting moiety.

Pyrithiones have been employed as as active site coordinating groups in enzyme inhibitors (WO 2009/015203; Agrawal, A., et al., J. Med. Chem. 2009, 52, 1063-1074; Muthyala, R., et al., Bioorg. Med. Chem. Lett. 2014, 24, 2535-2538; Muthyala, R., et al., Bioorg. Med. Chem. Lett. 2015, 25, 4320-4324; WO 2017/017631; Shin, W. S., et al., Chem. Med. Chem. 2017, 12, 845-849; US 2018/0369217).

Pyrithiones have also been incorporated into chelating agents (Abu-Dari, K.; Raymond, K. N. J. Inorg. Chem. 1991, 30, 519-524; Abu-Dari, K.; Raymond, K. N. J. Coord. Chem. 1992, 26, 1-14; Abu-Dari, K.; Karpishin, T. B.; Raymond, K. N. J. Inorg. Chem. 1993, 32, 3052-3055; (Magda, D., et al., “Synthesis and Anticancer Properties of Water-Soluble Zinc Ionophores,” Cancer Res. 2008, 68, 5318-5325; US 2013/8507531, Water Soluble Zinc Ionophores, Zinc Chelators, and/or Zinc Complexes and Use for Treating Cancer; US 12/140034 Thiohydroxamates as Inhibitors of Histone Deacetylase).

There is an ever-increasing need for metal chelate-based therapeutics and diagnostics, particularly targeted therapeutics and diagnostics. The present invention addresses this need.

### SUMMARY OF THE INVENTION

There are several factors to be considered in the design for a chelating agent for anticancer therapy. Some of the key issues apart from the kinetics are the high affinity for the target metal (such as a “soft ion”) which at the same time needs to have a low exchange rate for other biologically significant metal ions. In the compounds of the invention, the electronic properties of the target metal and ligand are considered and matched, providing a novel class of metal chelates in which a soft Lewis acid metal is complexed by a compatible organic ligand. The chelate should also be able to assume the appropriate coordination cavity size and geometry for the desired metal. In an exemplary embodiment, the metal ion is an ion of copper, a “soft” cation. Hence, copper prefers “soft” electron donors such as sulfur.

For agents having a targeting moiety conjugated thereto, the conditions under which the chelating agent is complexed with the metal ion is also an important factor. For instance, chelating agents that only complex useful amounts of a soft Lewis metal ion upon heating and/or prolonged exposure of the complexation mixture to high temperatures are non-ideal as the targeting agents (e.g. antibodies, antibody fragments, etc.) are generally sensitive to heat and other extreme conditions, will degrade and lose their specificity for their intended target. Addressing this problem, exemplary compounds of the invention complex soft Lewis acid metal ions under gentle conditions compatible with the presence of a targeting moiety and retain the target specificity and avidity of the targeting agent.

According to various embodiments of the present invention, metal chelating agents based on a sulfur-containing chelating moiety are provided. In various embodiments, the present invention provides chelators incorporating pyrithiones or cyclic thiohydroxamic acids as the chelating moiety.

In various embodiments, the agents are of use in radiopharmaceutical applications such as targeted cancer imaging and/or therapy.

Exempary metal chelates of the invention incorporate metal ions such as copper (e.g., copper-64 and copper-67), lead (e.g., lead-212), gallium(III), actinium(III), cobalt(II), manganese(III), iron(III), indium(III) and lanthanides.

### DESCRIPTION OF THE DRAWINGS

FIG. 1. Stability testing of macrocyclic copper chelates. FIG. 1A: HPLC traces from compound 27 before and after DTPA treatment. FIG. 1B: HPLC traces from compound 39 before and after DTPA treatment. FIG. 1C: HPLC traces from compound 44 before and after DTPA treatment.

### DESCRIPTION OF EMBODIMENTS

### 1. Definitions

Where substituent groups are specified by their conventional chemical formulae, written from left to right, they optionally equally encompass the chemically identical substituents, which would result from writing the structure from right to left, e.g., —CH₂O— optionally also recites —OCH2—.

The term “alkyl”, by itself or as part of another substituent, means a straight or branched chain hydrocarbon, which may be fully saturated, mono- or polyunsaturated and includes mono-, di- and multivalent radicals. Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds (i.e., alkenyl and alkynyl moieties). Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl” can refer to “alkylene”, which by itself or as part of another substituent means a divalent radical derived from an alkane, as exemplified, but not limited, by —CH₂CH₂CH₂CH₂—. Typically, an alkyl (or alkylene) group will have from 1 to 30 carbon atoms. A “lower alkyl” or “lower alkylene” is a shorter chain alkyl or alkylene group, generally having eight or fewer carbon atoms. In some embodiments, alkyl refers to an alkyl or combination of alkyls selected from C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉ and C₃₀ alkyl. In some embodiments, alkyl refers to C₁-C₂₅ alkyl. In some embodiments, alkyl refers to C₁-C₂₀ alkyl. In some embodiments, alkyl refers to C₁-C₁₅ alkyl. In some embodiments, alkyl refers to C₁-C₁₀ alkyl. In some embodiments, alkyl refers to C₁-C₆ alkyl.

The term “heteroalkyl,” by itself or in combination with another term, means an alkyl in which one or more carbons are replaced with one or more heteroatoms selected from the group consisting of O, N, Si and S, (preferably O, N and S), wherein the nitrogen and sulfur atoms may optionally be oxidized and the nitrogen heteroatom may optionally be quaternized. The heteroatoms O, N, Si and S may be placed at any interior position of the heteroalkyl group or at the position at which the alkyl group is attached to the remainder of the molecule. In some embodiments, depending on whether a heteroatom terminates a chain or is in an interior position, the heteroatom may be bonded to one or more H or substituents such as (C₁, C₂, C₃, C₄, C₅ or C₆) alkyl according to the valence of the heteroatom. Examples of heteroalkyl groups include, but are not limited to, —CH₂—CH₂—O—CH₃, —CH₂—CH₂—NH—CH₃, —CH2—CH₂—N(CH₃)—CH₃, —CH₂—S—CH₂—CH₃, —CH₂—CH₂, —S(O)—CH₃, —CH₂—CH₂—S(O)2—CH₃, —CH═CH—O—CH₃, —Si(CH₃)₃, —CH₂—CH═N—OCH₃, and —CH═CH—N(CH₃)—CH₃. No more than two heteroatoms may be consecutive, as in, for example, —CH₂—NH—OCH3 and —CH₂—O —Si(CH₃)₃, and in some instances, this may place a limit on the number of heteroatom substitutions. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. The designated number of carbons in heteroforms of alkyl, alkenyl and alkynyl includes the heteroatom count. For example, a (C₁, C₂, C₃, C₄, C₅ or C₆) heteroalkyl will contain, respectively, 1, 2, 3, 4, 5 or 6 atoms selected from C, N, O, Si and S such that the heteroalkyl contains at least one C atom and at least one heteroatom, for example 1-5 C and 1 N or 1-4 C and 2 N. Further, a heteroalkyl may also contain one or more carbonyl groups. In some embodiments, a heteroalkyl is any C₂-C₃₀ alkyl, C₂-C₂₅ alkyl, C₂-C₂₀ alkyl, C₂-C₁₅ alkyl, C₂-C₁₀ alkyl or C₂-C₆ alkyl in any of which one or more carbons are replaced by one or more heteroatoms selected from O, N, Si and S (or from O, N and S). In some embodiments, each of 1, 2, 3, 4 or 5 carbons is replaced with a heteroatom. The terms “alkoxy,” “alkylamino” and “alkylthio” (or thioalkoxy) are used in their conventional sense, and refer to those alkyl and heteroalkyl groups attached to the remainder of the molecule via an oxygen atom, a nitrogen atom (e.g., an amine group), or a sulfur atom, respectively.

The terms “cycloalkyl” and “heterocycloalkyl”, by themselves or in combination with other terms, refer to cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

The term “aryl” means a polyunsaturated, aromatic substituent that can be a single ring or optionally multiple rings (preferably 1, 2 or 3 rings) that are fused together or linked covalently. In some embodiments, aryl is a 3, 4, 5, 6, 7 or 8 membered ring, which is optionally fused to one or two other 3, 4, 5, 6, 7 or 8 membered rings. The term “heteroaryl” refers to aryl groups (or rings) that contain 1, 2, 3 or 4 heteroatoms selected from N, O, and S, wherein the nitrogen and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl.

In some embodiments, any of alkyl, heteroalkyl, cycloalkyl, heterocycloalkyl, aryl and heteroaryl is optionally substituted. That is, in some embodiments, any of these groups is substituted or unsubstituted. In some embodiments, substituents for each type of radical are selected from those provided below.

Substituents for the alkyl, heteroalkyl, cycloalkyl and heterocycloalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) are generically referred to as “alkyl group substituents”. In some embodiments, an alkyl group substituent is selected from -halogen, —OR′, ═O, ═NR′, ═N—OR′, —NR′R″, —SR′, —SiR′R″R″′, —OC(O)R′, —C(O)R′, —CO₂R′, —CONR′R″, —OC(O)NR′R″, —NR″C(O)R′, —NR′—C(O)NR″R″′, —NR″C(O)₂R′, —NR—C(NR′R″R′″)═NR″″, —NR—C(NR′R″)═NR ′″, —S(O)R′, —S(O)₂R′, —S(O)₂NR′R″, —NRSO₂R′, —CN and —NO₂ in a number ranging from zero to (2m′+1), where m′ is the total number of carbon atoms in such radical. In one embodiment, R′, R″, R″′ and R″″ are each independently selected from hydrogen, alkyl (e.g., C₁, C₂, C₃, C₄, C₅ and C₆ alkyl). In one embodiment, R′, R″, R″′ and R″″ each independently refer to hydrogen, substituted or unsubstituted heteroalkyl, substituted or unsubstituted aryl, e.g., aryl substituted with 1-3 halogens, substituted or unsubstituted alkyl, alkoxy or thioalkoxy groups, or arylalkyl groups. In one embodiment, R′, R″, R″′ and R″″ are each independently selected from hydrogen, alkyl, heteroalkyl, cycloalkyl, heterocycloalkyl, aryl, heteroaryl, alkoxy, thioalkoxy groups, and arylalkyl. When R′ and R″ are attached to the same nitrogen atom, they can be combined with the nitrogen atom to form a 5-, 6-, or 7-membered ring. For example, —NR′R″ can include 1-pyrrolidinyl and 4-morpholinyl. In some embodiments, an alkyl group substituent is selected from substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl.

Similar to the substituents described for the alkyl radical, substituents for the aryl and heteroaryl groups are generically referred to as “aryl group substituents”. In some embodiments, an aryl group substituent is selected from -halogen, —OR′, ═O, ═NR′, ═N—OR′, —NR′R″, —SR′, —SiR'R″R″′, —OC(O)R′, —C(O)R′, —CO₂R′, —CONR′R″, —OC(O)NR′R″, —NR″C(O)R′, —NR′—C(O)NR″R″′, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system. In some embodiments, R′, R″, R″′ and R″″ are independently selected from hydrogen and alkyl (e.g., C₁, C₂, C₃, C₄, C₅ and C₆ alkyl). In some embodiments, R′, R″, R″′ and R″″ are independently selected from hydrogen, substituted or unsubstituted alkyl, substituted or unsubstituted heteroalkyl, substituted or unsubstituted aryl and substituted or unsubstituted heteroaryl. In some embodiments, R′, R″, R″′ and R″″ are independently selected from hydrogen, alkyl, heteroalkyl, aryl and heteroaryl. In some embodiments, an aryl group substituent is selected from substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl.

Two of the substituents on adjacent atoms of the aryl or heteroaryl ring may optionally be replaced with a substituent of the formula —T—C(O)—(CRR′)_{q}—U—, wherein T and U are independently —NR—, —O—, —CRR′— or a single bond, and q is an integer of from 0 to 3. Alternatively, two of the substituents on adjacent atoms of the aryl or heteroaryl ring may optionally be replaced with a substituent of the formula —A—(CH₂)ᵣ—B—, wherein A and B are independently —CRR′—, —O—, —NR—, —S—, —S(O)—, —S(O)₂—, —S(O)₂NR′— or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X—(CR″R′″)_{d}—, where s and d are independently integers of from 0 to 3, and X is —O—, —NR′—, —S—, —S(O)—, —S(O)₂—, or —S(O)₂NR′—. The substituents R, R′, R″ and R′″ are preferably independently selected from hydrogen or substituted or unsubstituted (C₁-C₆)alkyl.

The term “acyl” refers to a species that includes the moiety —C(O)R, where R has the meaning defined herein. Exemplary species for R include H, halogen, substituted or unsubstituted alkyl, substituted or unsubstituted aryl, substituted or unsubstituted heteroaryl, and substituted or unsubstituted heterocycloalkyl. In some embodiments, R is selected from H and (C₁-C₆)alkyl.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is mean to include, but not be limited to, trifluoromethyl, 2,2,2-trifluoroethyl, 4-chlorobutyl, 3-bromopropyl, and the like. In some embodiments, halogen refers to an atom selected from F, Cl and Br.

The term “heteroatom” includes oxygen (O), nitrogen (N), sulfur (S) and silicon (Si). In some embodiments, a heteroatom is selected from N and S. In some embodiments, the heteroatom is O.

Unless otherwise specified, the symbol “R” is a general abbreviation that represents a substituent group that is selected from acyl, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. When a compound includes more than one R, R′, R″, R′″ and R″″ group, they are each independently selected.

For groups with solvent exchangeable protons, the ionized form is equally contemplated. For example, —COOH also refers to —COO⁻ and —OH also refers to —O⁻.

Any of the compounds disclosed herein can be made into a pharmaceutically acceptable salt. The term “pharmaceutically acceptable salts” includes salts of compounds that are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., Journal of Pharmaceutical Science, 66: 1-19 (1977)). Certain specific compounds of the present invention contain both basic and acidic functionalities that allow the compounds to be converted into either base or acid addition salts. The neutral forms of the compounds are preferably regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

In addition to salt forms, the present invention provides any of the compounds disclosed herein in a prodrug form. Prodrugs of the compounds described herein are those compounds that readily undergo chemical changes under physiological conditions to provide the compounds of the present invention.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

The compounds of the present invention may also contain unnatural proportions of atomic isotopes at one or more of the atoms that constitute such compounds. For example, the compounds may be labeled with deuterium (²H) or radiolabeled with radioactive isotopes, such as for example tritium (³H), iodine-125 (¹²⁵I) or carbon-14 (¹⁴C). All isotopic variations of the compounds of the present invention, whether radioactive or not, are intended to be encompassed within the scope of the present invention.

The symbol [symbol] , displayed perpendicular to a bond, indicates the point at which the displayed moiety is attached to the remainder of the molecule.

In some embodiments, the definition of terms used herein is according to IUPAC.

### 2. Compositions

The invention provides numerous compounds (ligands) and metal ion complexes thereof. Generally, a ligand comprises a plurality of chelating moieties that are linked together by way of a scaffold moiety.

There are several factors to be considered in the design for a chelating agent for anticancer therapy. Some of the key issues apart from the kinetics will be the high affinity for the target metal (such as a “soft ion”) which at the same time needs to have a low exchange rate for other biologically significant metal ions. In the compounds of the invention, the electronic properties of the target metal and ligand are considered and matched. The chelate should also be able to assume the appropriate coordination cavity size and geometry for the desired metal. In an exemplary embodiment, the metal ion is an ion of copper, a “soft” cation. Hence, copper prefers “soft” electron donors such as sulfur.

A ligand can comprise numerous chelating moieties. Particularly useful ligands contain a number of chelating moieties sufficient to provide, for example, 4, 6, 8 or 10 heteroatoms such as sulfur that coordinate with a metal ion to form a complex. The heteroatoms such as sulfur provide electron density for forming coordinate bonds with a positively charged ion, and such heteroatoms can thus be considered “donors”. In some embodiments, the plurality of chelating moieties of a ligand comprises a plurality of sulfur donors and a metal ion (such as a radionuclide) is chelated to the ligand via at least one of the sulfur donors. In some embodiments, a ligand comprises a plurality of sulfur donors and a metal ion (such as a radionuclide) is chelated to the ligand via a plurality or all of the sulfur donors.

In various embodiments, the metal chelating agents of the invention are able to complex soft Lewis acid metal ions without resort to extreme conditions. Thus, in an exemplary embodiment, in a complexing mixture containing X moles of chelating agent and at least a stoichiometric equivalent of X of the soft Lewis acid metal ion, following reaction between the chelating agent and the soft Lewis acid metal ion, at least about 0.6×, 0.7×, 0.8×, 0.9×, 0.94× or 0.98× of the chelating agent will be complexed with the soft Lewis acid metal ion. In an exemplary embodiment, the above level of complexation occurs at a reaction temperature of no more than about 15° C., no more than about 25° C., no more than about 35° C. or no more than about 45° C.

### Chelating Agents

In one aspect, the invention provides a chelating agent having the structure:

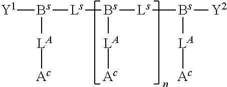

wherein n is an integer selected from 0, 1, 2, 3, and 4; Y¹ and Y² are independently selected from H, substituted or unsubstituted alkyl, and substituted or unsubstituted heteroalkyl; each B⁵ is independently selected from N, CR^{s}, B, SiR^{s}, and P; wherein R^{s} is selected from H and unsubstituted C₁-C₃ alkyl; each L^{A} is independently selected from a bond, —C(O)—, substituted or unsubstituted alkyl, and substituted or unsubstituted heteroalkyl; each L^{s} is independently selected from substituted or unsubstituted alkyl and substituted or unsubstituted heteroalkyl; and each A^{c} is an independently selected chelating moiety. Chelating moieties are as defined herein.

Any of the combinations of n, Y¹, Y², B^{s}, L^{A}, L^{s}, and A^{c} are encompassed by this disclosure and specifically provided by the invention.

In some embodiments, each L A is independently selected from a bond, —C(O)—, —(CH₂)ₐC(O)—, and —O(CH₂)ₐC(O)—, wherein a is an integer selected from 1, 2, 3, 4, 5, and 6. In some embodiments, each L A is the same. In some embodiments, L A is —C(O)—.

In some embodiments, the chelating agent comprises a linker. In some embodiments, at least one Ls is substituted with a linker. Linkers are as defined herein. In some embodiments, the linker is attached to a reactive functional group or a targeting moiety. In some embodiments, the ligand comprises a targeting moiety.

In some embodiments, the ligand comprises one or more modifying moieties. The modifying moieties can be the same or different.

In some embodiments, the compound (ligand) has a structure selected from:

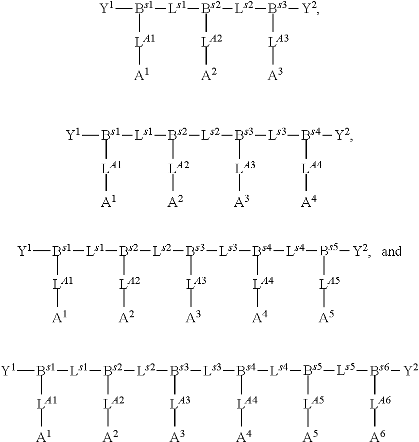

wherein Y¹ and Y² are independently selected from H, substituted or unsubstituted alkyl, and substituted or unsubstituted heteroalkyl; B^{s1}, B^{s2}, B^{s3}, B^{s4}, B^{s5}, and B^{s6} are independently selected from N, CR^{s}, B, SiR^{s}, and P; wherein R^{s} is selected from H and unsubstituted C₁-C₃ alkyl; L^{A1}, L^{A2}, L^{A3}, L^{A4}, L^{A5}, and L^{A6} are independently selected from a bond, —C(O)—, substituted or unsubstituted alkyl, and substituted or unsubstituted heteroalkyl; L^{s1}, L^{s2}, L^{s3}, L^{s4}, and L^{s5} are independently selected from substituted or unsubstituted alkyl and substituted or unsubstituted heteroalkyl, and A¹, A², A³, A⁴, A⁵, and A⁶ are independently selected chelating moieties.

Any of the combinations of Y¹, Y², B^{s1}, B^{s2}, B^{s3}, B^{s4}, B^{s5}, B^{s6}, L^{A1}, L^{A2}, L^{A3}, L^{A4}, L^{A5}, L^{A6}, L^{s1}, L^{s2}, L^{s3}, L^{s4}, L^{s5}, A¹, A², A³, A⁴, A⁵, and A⁶ are encompassed by this disclosure and specifically provided by the invention.

In some embodiments, at least one of L^{s1}, L^{s2}, L^{s3}, L^{s4}, and L^{s5} is substituted with a linker. In some embodiments, one, two, three, four, or five of L^{s1}, L^{s2}, L^{s3}, L^{s4}, and L^{s5} is/are substituted with a linker. In some embodiments, one of L^{s1}, L^{s2}, L^{s3}, L^{s4}, and L^{s5} is substituted with a linker. In some embodiments, two of L^{s1}, L^{s2}, L^{s3}, L^{s4}, and L^{s5} are substituted with a linker. In some embodiments, L^{s1} is substituted with a linker. In some embodiments, L^{s2} is substituted with a linker. In some embodiments, L^{s3} is substituted with a linker. In some embodiments, L^{s4} is substituted with a linker. In some embodiments, L^{s5} is substituted with a linker. Linkers are as defined herein.

In some embodiments, L^{s1}, L^{s2}, L^{s3}, L^{s4}, and L^{s5} are each independently selected from substituted or unsubstituted C₁-C₈ (i.e., C₁, C₂, C₃, C₄, C₅, C₆, C₇, C₈) alkyl and substituted or unsubstituted C₁-C₈ (i.e., C₁, C₂, C₃, C₄, C₅, C₆, C₇, C₈) heteroalkyl. In some embodiments, L^{s1}, L^{s2}, L^{s3}, L^{s4}, and L^{s5} are each independently selected from substituted or unsubstituted C₁, C₂, C₃, C₄, C₅, C₆, C₇ and C₈ alkyl. In some embodiments, L^{s1}, L^{s2}, L^{s3}, L^{s4}, and L^{s5} are each independently selected from substituted or unsubstituted C₁, C₂, C₃, C₄, C₅, C₆, C₇ and C₈ alkyl, wherein at least one of L^{s1}, L^{s2}, L^{s3}, L^{s4}, and L^{s5} is substituted with a linker. In some embodiments, L^{s1}, L^{s2}, L^{s3}, L^{s4}, and L^{s5} are each independently selected from substituted or unsubstituted C₂-C₅ alkyl. In some embodiments, L^{s1}, L^{s2}, L^{s3}, L^{s4}, and L^{s5} are each independently selected from substituted or unsubstituted C₂-C₅ alkyl, wherein at least one of L^{s1}, L^{s2}, L^{s3}, L^{s4}, and L^{s5} is substituted with a linker. In some embodiments, L^{s1}, L^{s2}, L^{s3}, L^{s4}, and L^{s5} are each independently selected from substituted or unsubstituted C₃-C₄ alkyl. In some embodiments, L^{s1}, L^{s2}, L^{s3}, L^{s4}, and L^{s5} are each independently selected from substituted or unsubstituted C₃-C₄ alkyl, wherein at least one of L^{s1}, L^{s2}, L^{s3}, L^{s4}, and L^{s5} is substituted with a linker.

In some embodiments, B^{s1}, B^{s2}, B^{s3}, B^{s4}, B^{s5}, and B^{s6} are N.

In some embodiments, L^{A1}, L^{A2}, L^{A3}, L^{A4}, L^{A5}, and L^{A6} are independently selected from a bond, —C(O)—, —(CH₂)ₐC(O)—, and —O(CH₂)ₐC(O)—, wherein a is an integer selected from 1, 2, 3, 4, 5, and 6. In some embodiments, L^{A1}, L^{A2}, L^{A3}, L^{A4}, L^{A5}, and L^{A6} are the same. In some embodiments, L^{A1}, L^{A2}, L^{A3}, L^{A4}, L^{A5}, and L^{A6} are —C(O)—.

In some embodiments, Y¹ and Y² are H.

### Chelating Moieties

In various embodiments, the chelating agents of the compounds of the invention include a first chelating moiety according to Formula I:

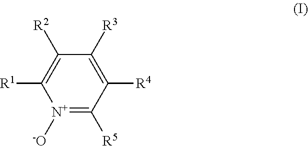

wherein R¹, R², R³, and R⁴ are independently selected from a bond, H, substituted or unsubstituted alkyl, substituted or unsubstituted heteroalkyl, halogen, CN, —CF₃, —C(O)R¹⁰, —SO₂NR¹⁰R¹¹, —NR¹⁰R¹¹, —OR¹⁰, —S(O)₂R¹⁰, —COOR¹⁰, —S(O)₂OR¹⁰, —OC(O)R¹⁰, —C(O)NR¹⁰R¹¹, —(CH₂)ₘC(O)NR¹⁰R¹¹, —O(CH₂)ₘC(O)NR¹⁰R¹¹, —NR¹⁰C(O)R¹¹, —NR¹⁰SO₂R¹¹, and —NO₂. R⁵ is SH, S—, OH or O—. R¹⁰ and R¹¹ and are independently selected from H, substituted or unsubstituted alkyl, substituted or unsubstituted heteroalkyl, substituted or unsubstituted aryl, substituted or unsubstituted heteroaryl and substituted or unsubstituted heterocycloalkyl; and R¹⁰ and R¹¹, together with the atoms to which they are attached, are optionally joined to form a 5-, 6- or 7-membered ring selected from substituted or unsubstituted cycloalkyl, substituted or unsubstituted heterocycloalkyl, substituted or unsubstituted aryl and substituted or unsubstituted heteroaryl. The index m is selected from 1, 2, 3, 4, 5, 6, 7, 8, 9, and 10.

In various embodiments, the chelating agents of the invention include 2, 3, 4, 5, 6 or more of the chelating moieites according to Formula I. The chelating moieties may be a component of a linear or a macrocyclic chelating agent. The substituents on each of the chelating moieties may be the same or different, and it is within the ability of one of ordinary skill in the art to select an appropriate combination of substituents to achieve the desired properties for the chelating agent.

It is understood that heteroatoms, displayed as protonated equally encompass these atoms in their deprotonated form and vice versa.

In an exemplary embodiment, one of R¹-R⁴ is a linker to a second chelating moiety according to Formula I. In an exemplary embodiment, the linker is substituted or unsubstituted alkyl or substituted or unsubstituted heteroalkyl.

In some embodiments, a compound of the invention has the structure according to Formula II:

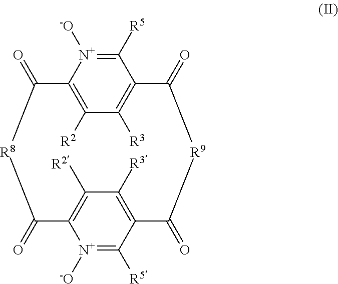

in which each R², R³, R⁵, R^{2′}, R^{3′} and R^{5═} are as described above for R¹ to R⁵. R⁸ and R⁹ are independently members selected from substituted or unsubstituted alkyl, and substituted or unsubstituted heteroalkyl. In an exemplary embodiment, one or more of R², R³, R^{1′}, R^{2′}, R^{3′}, R⁸ or R⁹ includes a linker to a reactive functional group or to a targeting moiety.

In various embodiments, the invention provides a compound having a structure according to Formula III:

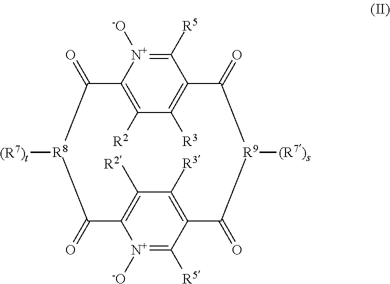

in which exemplary R⁷ and R^{7′} include 1, 2, 3, 4, 5, 6, 7, 8, 9, or 10 non-hydrogen atoms forming a linker linking a first Formula III to a second Formula III. The indices s and t are 0 or 1, with the proviso that at least one of s and t is 1 and, in an exemplary embodiment, s and t are not both 1. In various embodiments, R⁷ or R^{7′} includes a reactive functional group or targeting moiety pendent from the terminus or an interior position of the hydrocarbyl moiety. In various embodiments, R⁷ or R^{7′} is hydrocarbyl with a reactive functional group pendent from the terminus or an interior position of the hydrocarbyl moiety. In various embodiments, a member selected from R⁷ and R^{7′} includes one or more than one heteroatom. In various embodiments, each more than one heteroatom is the same heteroatom. In various embodiments, each more than one heteroatom is a different heteroatom.

In an exemplary embodiment, the compound of the invention has a structure according to Formula IV:

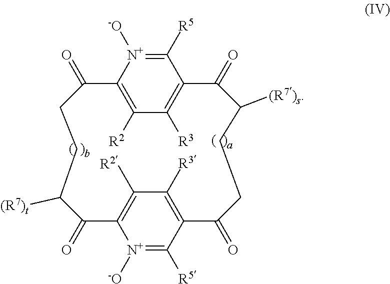

in which each previously described R group is as described above. R^{8′} and R^{9′} are independently members selected from substituted or unsubstituted alkyl, and substituted or unsubstituted heteroalkyl. The indices s and t are 0 or 1, with the proviso that at least one of s and t is 1 and, in an exemplary embodiment, s and t are not both 1. The indices a and b are independently selected from 1, 2, 3, 4, 5, and 6. In various embodiments, R⁷ or R^{7′} includes a reactive functional group or targeting moiety pendent from the terminus or an interior position of the hydrocarbyl moiety. In various embodiments, R⁷ or R^{7′} is hydrocarbyl with a reactive functional group pendent from the terminus or an interior position of the hydrocarbyl moiety. In various embodiments, a member selected from R⁷ and R^{7′} includes one or more than one heteroatom. In various embodiments, each more than one heteroatom is the same heteroatom. In various embodiments, each more than one heteroatom is a different heteroatom.

In various embodiments, a compound of the invention is a macrocycle as described above, and it has the structure according to Formula V:

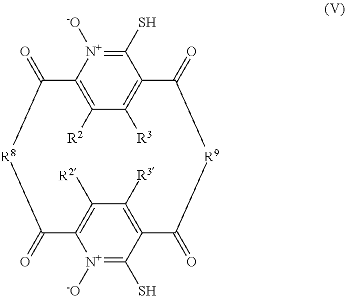

in which each R² and R³ is as described above, R⁸ and R⁹ are independently selected from substituted or unsubstituted alkyl or substituted or unsubstituted heteroalkyl. In an exemplary embodiment, one or more of R², R³, R⁸ or R⁹ includes a linker to a reactive functional group or to a targeting moiety. In various embodiments, R⁸ and R⁹ are independently selected diaminyl linkers. In various embodiments, R⁸ and R⁹ optionally include one or more than one heteroatom. In various embodiments, the more than one heteroatoms are the same heteroatom. In various embodiments, the more than one heteroatom are different heteroatoms.

In various embodiments, a compound of the invention has the structure according for Formula VI:

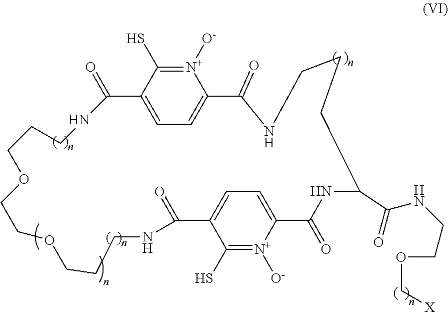

in which each index n is independently selected from the integers 0, 1, 2, 3, 4, 5, 6, 7, 8, 9, and 10; and X is a reactive functional group or a targeting moiety.

Higher order chelating agents are also provided by the invention. Thus, in exemplary embodiments, the invention provides chelating agents with 3, 4, 5, 6, 7, or 8 chelating moieties, which may be the same or different within the chelating agent.

In an exemplary embodiment, the chelating agent of the invention has a structure according to Formula VII:

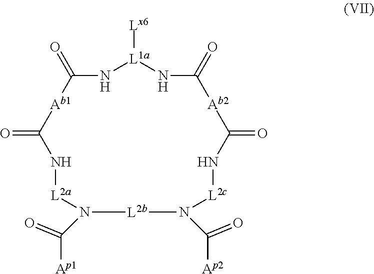

in which L^{1a}, L^{2a}, L^{2b}, L^{2c}, and L^{x6} are independently selected linkers as these groups are defined herein. The moieties A^{b1}, A^{b2}, A^{p1} and A^{p2} are independently selected chelating moieties as these groups are defined herein. These and other structures into which the chelating moieties set forth herein are readily incorporated are set forth in WO2019173639, the content of which is incorporated herein by reference in its entirety for all purposes.

In an exemplary embodiment, the invention provides a chelating agent according to Formula VIII:

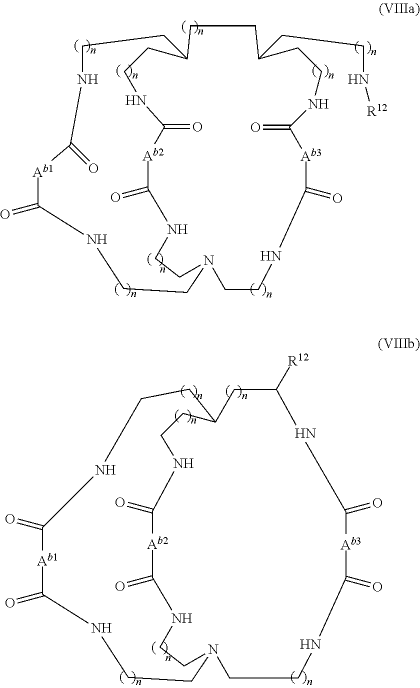

in which A^{b1}, A^{b2}, and A^{b3} are chelating moieties as described herein, and R¹² is a linker to a reactive functional group, a targeting moiety or a chelating moiety as described herein. In this exemplary Formula VIIIa, the scaffold is shown as comprising methylene and ethylene moieties bridging the various groups within the scaffold, however, as will be apparent to those of ordinary skill in the art from the specification and the examples, these bridging moieties are variable across Formula VIII and they can be zero-order, 1, 2, 3, 4, 5,or 6 member alkyl or heteroalyl moieties, e.g., C₁, C₂, C₃, C₄, C₅, or C₆ moieties, without limitation on their variation and permutations across the macrocycle structure. In an exemplary embodiment according to Formula VIIIb, each n is independently selected and is 0, 1, 2, 3, 4, 5, or 6. One or more of each of these bridging moieties is optionally substituted with a linker to a reactive functional group, a targeting moiety or another chelaing moiety (e.g., R¹²), and this linker can be substituted or unsubstituted alkyl or heteroalkyl. These and other structures into which the chelating moieties set forth herein are readily incorporated are set forth in US20170183365, the content of which is incorporated herein by reference in its entirety for all purposes.

In various embodiments, a compound of the invention is a linear chelating agent. In an exemplary embodiment, the chelating agent has the structure according to Formula IX:

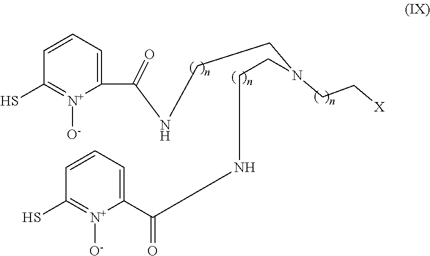

in which each index n is independently selected from 0, 1, 2, 3, 4, 5, 6, 7, 8, 9, and 10; and X is a reactive functional group or a targeting moiety.

In an exemplary embodiment, the chelating agent is chelated to a metal ion selected from an ion of Cu(II), Zn(II), Pb(II), Ga(III), ⁶⁴Cu(II), ⁶⁷Cu(II), ²¹²Pb(II), ⁶⁷Ga(III), ⁶⁸Ga(III), ⁶⁴Cu, ⁶⁷Cu, ²¹²Pb, ⁶⁷Ga, and ⁶⁸Ga.

As will be apparent to those of skill in the art, each of the chelating agents set forth herein can exist as a chelate of a metal ion, e.g., a soft Lewis acid metal ion.

### Linker to Functional/Targeting Moiety

A “linker”, “linking member”, or “linking moiety” as used herein is a moiety that joins or potentially joins, covalently or noncovalently, a first moiety to a second moiety. In particular, a linker attaches or can potentially attach a ligand described herein to another molecule, such as a targeting moiety. In some embodiments, a linker attaches or could potentially attach a ligand described herein to a solid support. A linker comprising a reactive functional group that can be further reacted with a reactive functional group on a structure of interest in order to attach the structure of interest to the linker is referred to as a “functionalized linker”. In exemplary embodiments, a linker is a functionalized linker. In exemplary embodiments, a ligand comprises one or more functionalized linkers. In some embodiments, a linker comprises a targeting moiety. In some embodiments, a linker to a targeting moiety comprises a bond to the targeting moiety.

A linker can be any useful structure for that joins a ligand to a reactive functional group or a targeting moiety, such as an antibody. Examples of a linker include 0-order linkers (i.e., a bond), substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. Further exemplary linkers include substituted or unsubstituted (C₁, C₂, C₃, C₄, C₅, C₆, C₇, C₈, C₉ or C₁₀) alkyl, substituted or unsubstituted heteroalkyl, —C(O)NR′—, —C(O)O—, —C(O)S—, and —C(O)CR′R″, wherein R′ and R″ are members independently selected from H, substituted or unsubstituted alkyl, substituted or unsubstituted heteroalkyl, substituted or unsubstituted aryl, substituted or unsubstituted heteroaryl and substituted or unsubstituted heterocycloalkyl. In some embodiments, a linker includes at least one heteroatom. Exemplary linkers also include —C(O)NH—, —C(O), —NH—, —S—, —O—, and the like. In an exemplary embodiment, a linker is a heteroalkyl substituted with a reactive functional group.

In some embodiments, a ligand comprises a linker to a solid support. That is, any linker described herein may be a linker comprising a reactive functional group that could react with a reactive functional group on a solid support to join the linker to the solid support. Any linker described herein may be a linker comprising a bond to a solid support. A “solid support” is any material that can be modified to contain discrete individual sites suitable for the attachment or association of a ligand. Suitable substrates include biodegradable beads, non-biodegradable beads, silica beads, magnetic beads, latex beads, glass beads, quartz beads, metal beads, gold beads, mica beads, plastic beads, ceramic beads, or combinations thereof. Of particular use are biocompatible polymers, including biodegradable polymers that are slowly removed from the system by enzymatic degradation. Example biodegradable materials include starch, cross-linked starch, poly(ethylene glycol), polyvinylpyrrolidine, polylactides (PLA), polyglycolides (PGA), poly(lactide-co-glycolides) (PLGA), polyanhydrides, polyorthoesters, poly(DTH iminocarbonate), poly(bisphenol A iminocarbonate), polycyanoacrylate, polyphosphazene, mixtures thereof and combinations thereof. Other suitable substances for forming the particles exist and can be used. In some embodiments, a solid support is a bead comprising a cross-linked starch, for example, cross-linked potato starch. Beads made from starch are completely biodegradable in the body, typically by serum amylase, a naturally occurring enzyme found in the body. In these embodiments, the ligand optionally further comprises a targeting moiety or a linker to a targeting moeity. In cases where a ligand that is attached to a solid support does not comprise a targeting moiety, the ligand can be localized directly by the practitioner, for example, by direct surgical implantation.

In some embodiments, a linker has the structure -L-X, wherein L is selected from a bond, acyl, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl; and X is selected from a reactive functional group, a protected functional group, or a targeting moiety.

In some embodiments, L is selected from substituted or unsubstituted alkyl and substituted or unsubstituted heteroalkyl. In some embodiments, L is heteroalkyl. In some embodiments, L is (C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉ or C₂₀) alkyl in which 1, 2 or 3 atoms are replaced with a heteroatom, such as nitrogen or oxygen. In some embodiments, L comprises a modifying moiety.

In some embodiments, X is selected from —NH₂, —C(O)OH, alkyl ester (e.g., methyl ester), N-hydroxysuccinimide (NETS) ester, sulfo-NHS ester, isothiocyanate, and maleimide. In some embodiments, X is selected from —NH₂ and —C(O)OH.

In some embodiments, -L-X is selected from

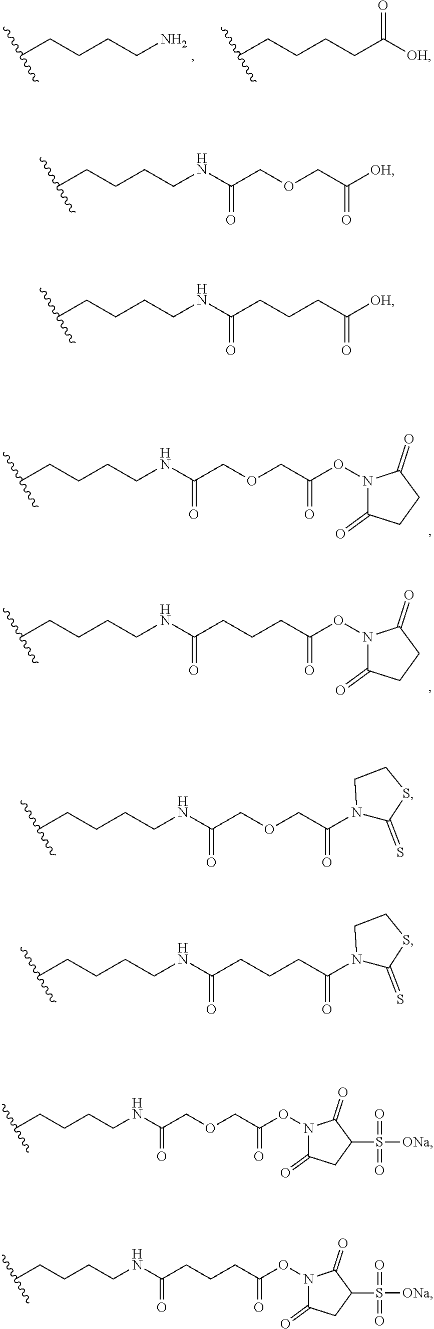

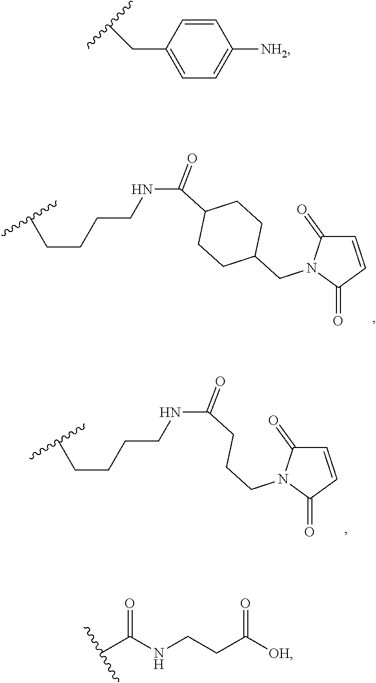 [SMILES: *Cc1ccc(N)cc1 ; *CCCCNC(=O)C1CCC(CN2C(=O)C=CC2=O)CC1 ; *CCCCNC(=O)CCCN1C(=O)C=CC1=O ; *C(=O)NCCC(=O)O]

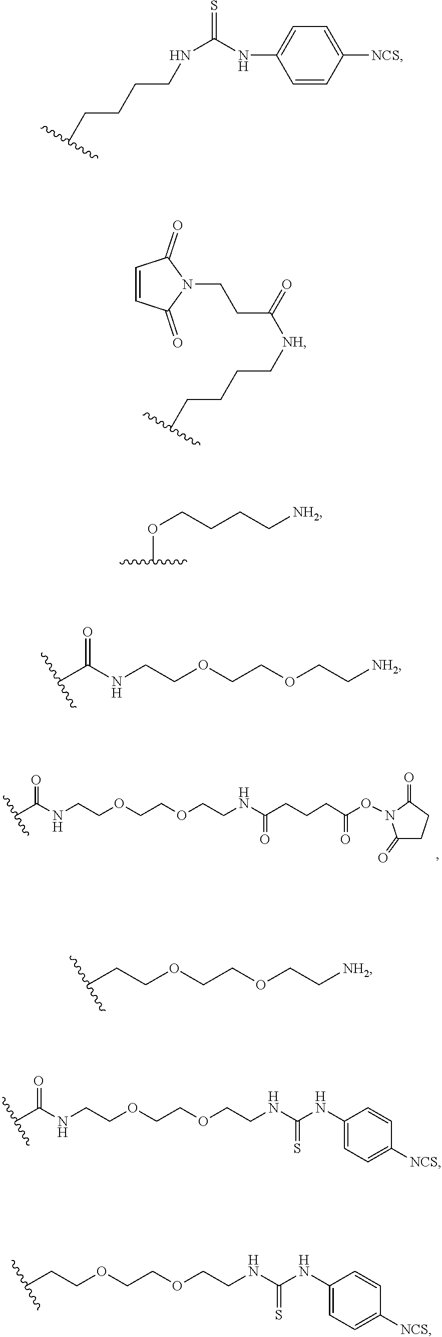

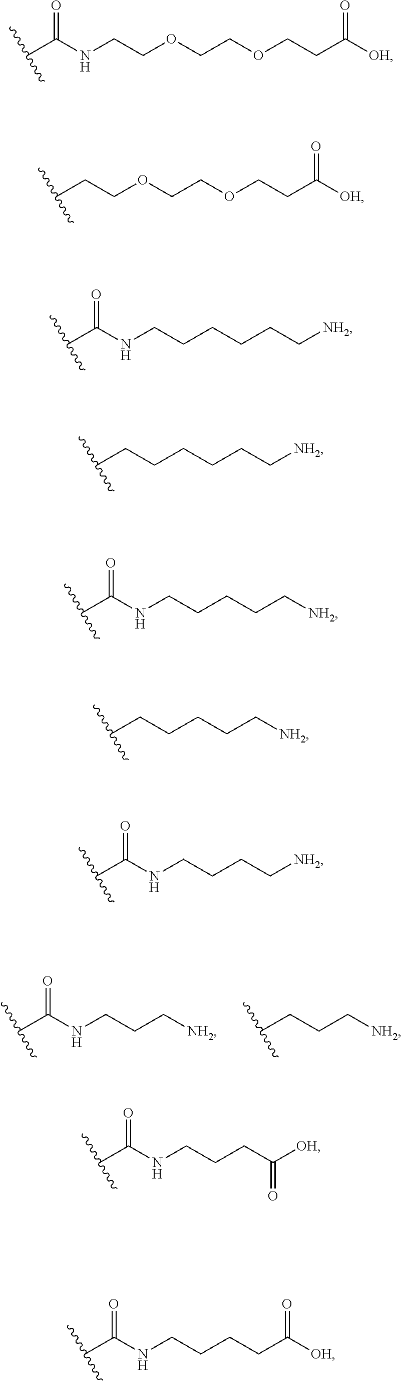

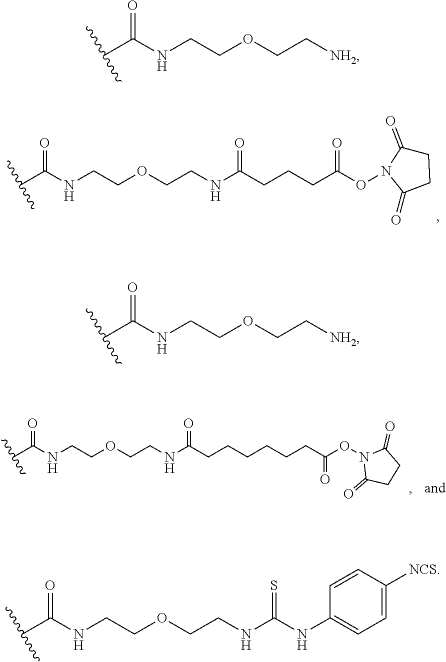

some embodiments, R^{L} comprises a modifying moiety.

In some embodiments, a linker and -L-X has a structure selected from:

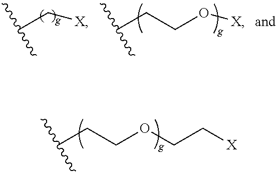

where g is an integer independently selected from 1, 2, 3, 4, 5, or 6, and X is a member selected from a reactive functional group and a targeting moiety.

In some embodiments, a linker has the structure:

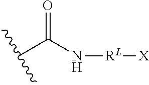

wherein R^{L} is selected from substituted or unsubstituted alkyl, and substituted or unsubstituted heteroalkyl; and X is as defined herein. In some embodiments, R^{L} is a substituted or unsubstituted alkoxyalkyl. In some embodiments, R L is a substituted or unsubstituted monoether. In some embodiments, R^{L} is a substituted or unsubstituted polyether. In some embodiments, the polyether has from 2 to 10 (i.e., 2, 3, 4, 5, 6, 7, 8, 9, or 10) ether groups. In some embodiments, R^{L} comprises a modifying moiety.

In some embodiments, a linker has a structure selected from:

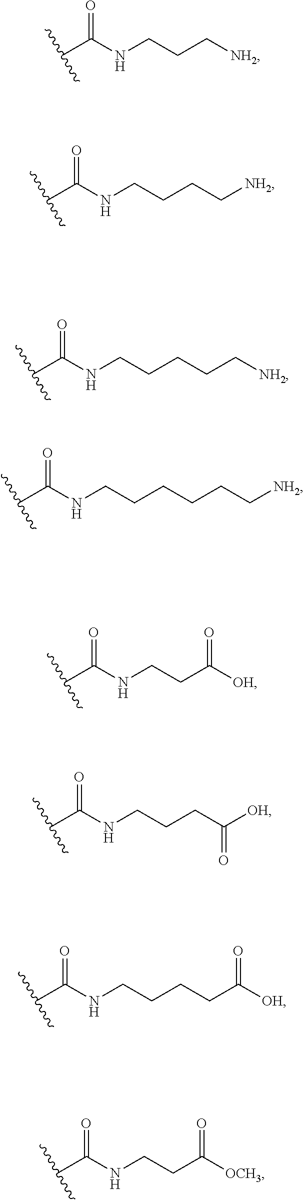

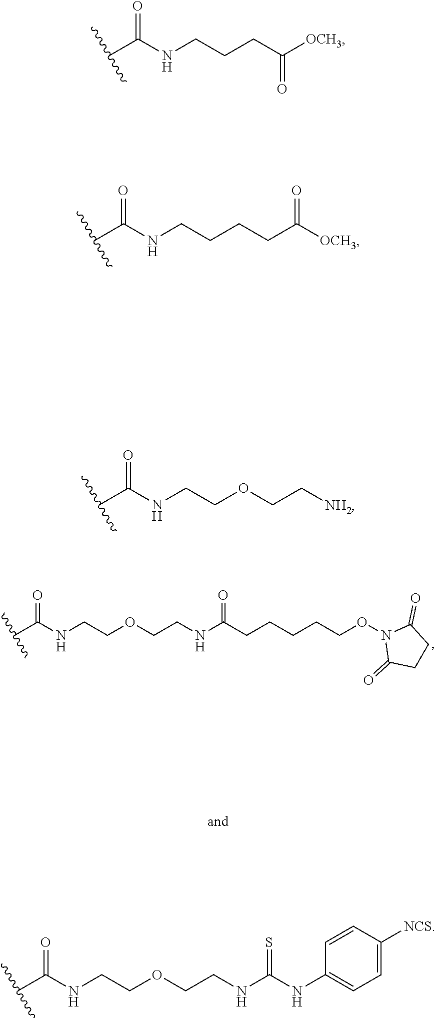

In some embodiments, a linker has a structure selected from:

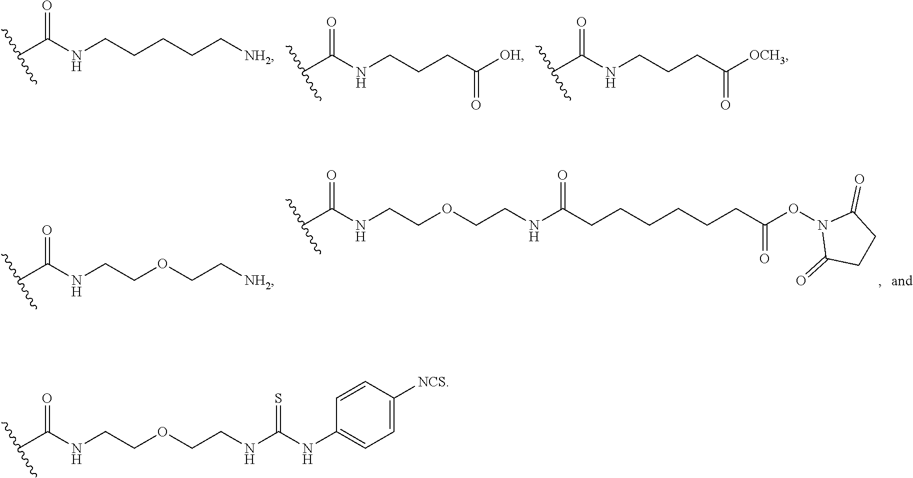

In exemplary embodiments, a linker is a linker to a targeting moiety. In some embodiments, the targeting moiety is selected from a polypeptide, a nucleic acid, a lipid, a polysaccharide, a small molecule, a cofactor and a hormone. In exemplary embodiments, the targeting moiety is an antibody or antibody fragment.

In a linker with multiple reactive functional groups, a particular functional group can be chosen such that it does not participate in, or interfere with, the reaction controlling the attachment of the functionalized spacer component to another ligand component. Alternatively, the reactive functional group can be protected from participating in the reaction by the presence of a protecting group. Those of skill in the art understand how to protect a particular functional group from interfering with a chosen set of reaction conditions. For examples of useful protecting groups, See Greene et al., PROTECTIVE GROUPS IN ORGANIC SYNTHESIS, John Wiley & Sons, New York, 1991.

### Targeting Moiety

In exemplary embodiments, a linker joins a ligand to a targeting moiety. That is, in exemplary embodiments, a linker comprises a targeting moiety. In some embodiments, a ligand comprises a linker to a targeting moiety. Any linker described herein may be a linker comprising a reactive functional group that could react with a reactive functional group on a targeting moiety to join the linker to the targeting moiety. Any linker described herein may be a linker comprising a bond to a targeting moiety. The term “targeting moiety” refers to a moiety serves to target or direct the molecule to which it is attached (e.g., a ligand or a ligand complexed to a metal ion (such as a radionuclide)) to a particular location or molecule. Thus, for example, a targeting moiety may be used to target a molecule to a specific target protein or enzyme, or to a particular cellular location, to a particular cell type or to a diseased tissue. As will be appreciated by those in the art, the localization of proteins within a cell is a simple method for increasing effective concentration. For example, shuttling an imaging agent and/or therapeutic into the nucleus confines them to a smaller space thereby increasing concentration. Finally, the physiological target may simply be localized to a specific compartment, and the agents must be localized appropriately.

The targeting moiety can be a small molecule (e.g., MW<500D), which includes both non-peptides and peptides. Examples of a targeting moiety also include peptides, polypeptides (including proteins, and in particular antibodies, which includes antibody fragments), nucleic acids, oligonucleotides, carbohydrates, lipids, hormones (including proteinaceous and steroid hormones (for instance, estradiol)), growth factors, lectins, receptors, receptor ligands, cofactors and the like. Targets of a targeting moiety can include a complementary nucleic acid, a receptor, an antibody, an antigen or a lectin, for example.

In exemplary embodiments, a targeting moiety can bind to a target with high binding affinity. In other words, a targeting moiety with high binding affinity to a target has a high specificity for or specifically binds to the target. In some embodiments, a high binding affinity is given by a dissociation constant K_{d} of about 10⁻⁷ M or less. In exemplary embodiments, a high binding affinity is given by a dissociation constant K_{d} of about 10⁻⁸ M or less, about 10⁻⁹ M or less, about 10⁻¹⁰ M or less, about 10⁻¹¹ M or less, about 10⁻¹² M or less, about 10⁻¹³ M or less, about 10⁻¹⁴ M or less or about 10⁻¹⁵ M or less. A compound may have a high binding affinity for a target if the compound comprises a portion, such as a targeting moiety, that has a high binding affinity for the target.

In exemplary embodiments, a targeting moiety is an antibody. An “antibody” refers to a protein comprising one or more polypeptides substantially encoded by all or part of the recognized immunoglobulin genes. The recognized immunoglobulin genes, for example in humans, include the kappa (κ), lambda (λ) and heavy chain genetic loci, which together compose the myriad variable region genes, and the constant region genes mu (μ), delta (δ), gamma (γ), epsilon (ε) and alpha (α), which encode the IgM, IgD, IgG, IgE, and IgA isotypes respectively. Antibody herein is meant to include full length antibodies and antibody fragments, and may refer to a natural antibody from any organism, an engineered antibody or an antibody generated recombinantly for experimental, therapeutic or other purposes as further defined below. Antibody fragments include Fab, Fab′, F(ab′)₂, Fv, scFv or other antigen-binding subsequences of antibodies and can include those produced by the modification of whole antibodies or those synthesized de novo using recombinant DNA technologies. The term “antibody” refers to both monoclonal and polyclonal antibodies. Antibodies can be antagonists, agonists, neutralizing, inhibitory or stimulatory.

In an exemplary embodiment, the targeting moiety is a peptide. An exemplary peptide is the decameric peptide for D-Lys6-gonadotropin releasing hormone (Glp-His-Trp-Ser-Tyr-Lys-Leu-Arg-Pro-Gly-NH₂).

While a targeting moiety may be appended to a ligand in order to localize the compound to a specific region in an animal, certain ligands have a natural affinity for cells, tissue, organs or some other part of the animal. For example, a ligand disclosed herein might have a natural or intrinsic affinity for bone. Thus, in some embodiments, a ligand does not comprise a targeting moiety or a linker to a targeting moiety. A ligand lacking a targeting moiety can be used in any method that does not require specific targeting.

### Reactive Functional Groups

In one embodiment, a linker comprises a reactive functional group (or a “reactive functional moiety”, used synonymously), which can be further reacted to covalently attach the linker to a targeting moiety. Reactive functional groups and classes of reactions useful in practicing the present invention are generally those that are well known in the art of bioconjugate chemistry. Currently favored classes of reactions available with reactive functional groups of the invention are those which proceed under relatively mild conditions. These include, but are not limited to nucleophilic substitutions (e.g., reactions of amines and alcohols with acyl halides and activated esters), electrophilic substitutions (e.g., enamine reactions) and additions to carbon-carbon and carbon-heteroatom multiple bonds (e.g., Michael reactions and Diels-Alder reactions). These and other useful reactions are discussed, for example, in March, Advanced Organic Chemistry (3rd Ed., John Wiley & Sons, New York, 1985); Hermanson, Bioconjugate Techniques (Academic Press, San Diego, 1996); and Feeney et al., Modification of Proteins, Advances in Chemistry Series, Vol. 198 (American Chemical Society, Washington, D.C., 1982).

In some embodiments, a reactive functional group refers to a group selected from olefins, acetylenes, alcohols, phenols, ethers, oxides, halides, aldehydes, ketones, carboxylic acids, esters, amides, cyanates, isocyanates, thiocyanates, isothiocyanates, amines, hydrazines, hydrazones, hydrazides, diazo, diazonium, nitro, nitriles, mercaptans, sulfides, disulfides, sulfoxides, sulfones, sulfonic acids, sulfinic acids, acetals, ketals, anhydrides, sulfates, sulfenic acids isonitriles, amidines, imides, imidates, nitrones, hydroxylamines, oximes, hydroxamic acids thiohydroxamic acids, allenes, ortho esters, sulfites, enamines, ynamines, ureas, pseudoureas, semicarbazides, carbodiimides, carbamates, imines, azides, azo compounds, azoxy compounds, and nitroso compounds. Reactive functional groups also include those used to prepare bioconjugates, e.g., N-hydroxysuccinimide esters, maleimides and the like. Methods to prepare each of these functional groups are well known in the art and their application or modification for a particular purpose is within the ability of one of skill in the art (see, for example, Sandler and Karo, eds., Organic Functional Group Preparations, (Academic Press, San Diego, 1989)).

A reactive functional group can be chosen according to a selected reaction partner. As an example, an activated ester, such as an NHS ester will be useful to label a protein via lysine residues. Sulfhydryl reactive groups, such as maleimides can be used to label proteins via amino acid residues carrying an SH-group (e.g., cysteine). Antibodies may be labeled by first oxidizing their carbohydrate moieties (e.g., with periodate) and reacting resulting aldehyde groups with a hydrazine containing ligand.

The reactive functional groups can be chosen such that they do not participate in, or interfere with, the reactions necessary to assemble the reactive ligand. Alternatively, a reactive functional group can be protected from participating in the reaction by means of a protecting group. Those of skill in the art understand how to protect a particular functional group so that it does not interfere with a chosen set of reaction conditions. For examples of useful protecting groups, see, for example, Greene et al., PROTECTIVE GROUPS IN ORGANIC SYNTHESIS, John Wiley & Sons, New York, 1991.

### Amines and Amino-Reactive Groups

In one embodiment, a reactive functional group is selected from an amine, (such as a primary or secondary amine), hydrazine, hydrazide and sulfonylhydrazide. Amines can, for example, be acylated, alkylated or oxidized. Useful non-limiting examples of amino-reactive groups include N-hydroxysuccinimide (NETS) esters, sulfur-NETS esters, imidoesters, isocyanates, isothiocyanates, acylhalides, arylazides, p-nitrophenyl esters, aldehydes, sulfonyl chlorides, thiazolides and carboxyl groups.

NETS esters and sulfo-NHS esters react preferentially with a primary (including aromatic) amino groups of a reaction partner. The imidazole groups of histidines are known to compete with primary amines for reaction, but the reaction products are unstable and readily hydrolyzed. The reaction involves the nucleophilic attack of an amine on the acid carboxyl of an NETS ester to form an amide, releasing the N-hydroxysuccinimide.

Imidoesters are the most specific acylating reagents for reaction with amine groups of a molecule such as a protein. At a pH between 7 and 10, imidoesters react only with primary amines. Primary amines attack imidates nucleophilically to produce an intermediate that breaks down to amidine at high pH or to a new imidate at low pH. The new imidate can react with another primary amine, thus crosslinking two amino groups, a case of a putatively monofunctional imidate reacting bifunctionally. The principal product of reaction with primary amines is an amidine that is a stronger base than the original amine. The positive charge of the original amino group is therefore retained. As a result, imidoesters do not affect the overall charge of the conjugate.

Isocyanates (and isothiocyanates) react with the primary amines of the conjugate components to form stable bonds. Their reactions with sulfhydryl, imidazole, and tyrosyl groups give relatively unstable products.

Acylazides are also used as amino-specific reagents in which nucleophilic amines of the reaction partner attack acidic carboxyl groups under slightly alkaline conditions, e.g. pH 8.5.

Arylhalides such as 1,5-difluoro-2,4-dinitrobenzene react preferentially with the amino groups and tyrosine phenolic groups of the conjugate components, but also with its sulfhydryl and imidazole groups.

p-Nitrophenyl esters of carboxylic acids are also useful amino-reactive groups. Although the reagent specificity is not very high, α- and ε-amino groups appear to react most rapidly.

Aldehydes react with primary amines of the conjugate components (e.g., ε-amino group of lysine residues). Although unstable, Schiff bases are formed upon reaction of the protein amino groups with the aldehyde. Schiff bases, however, are stable, when conjugated to another double bond. The resonant interaction of both double bonds prevents hydrolysis of the Schiff linkage. Furthermore, amines at high local concentrations can attack the ethylenic double bond to form a stable Michael addition product. Alternatively, a stable bond may be formed by reductive amination.

Aromatic sulfonyl chlorides react with a variety of sites of the conjugate components, but reaction with the amino groups is the most important, resulting in a stable sulfonamide linkage.

Free carboxyl groups react with carbodiimides, soluble in both water and organic solvents, forming pseudoureas that can then couple to available amines yielding an amide linkage. Yamada et al., Biochemistry, 1981, 20: 4836-4842, e.g., teach how to modify a protein with carbodiimides.

### Sulfhydryl and Sulfhydryl-Reactive Groups

In another embodiment, a reactive functional group is selected from a sulfhydryl group (which can be converted to disulfides) and sulfhydryl-reactive group. Useful non-limiting examples of sulfhydryl-reactive groups include maleimides, alkyl halides, acyl halides (including bromoacetamide or chloroacetamide), pyridyl disulfides, and thiophthalimides.

Maleimides react preferentially with the sulfhydryl group of the conjugate components to form stable thioether bonds. They also react at a much slower rate with primary amino groups and the imidazole groups of histidines. However, at pH 7 the maleimide group can be considered a sulfhydryl-specific group, since at this pH the reaction rate of simple thiols is 1000-fold greater than that of the corresponding amine.

Alkyl halides react with sulfhydryl groups, sulfides, imidazoles, and amino groups. At neutral to slightly alkaline pH, however, alkyl halides react primarily with sulfhydryl groups to form stable thioether bonds. At higher pH, reaction with amino groups is favored.

Pyridyl disulfides react with free sulfhydryl groups via disulfide exchange to give mixed disulfides. As a result, pyridyl disulfides are relatively specific sulfhydryl-reactive groups.

Thiophthalimides react with free sulfhydryl groups to also form disulfides.

### Other Reactive Functional Groups

Other exemplary reactive functional groups include:
(i) carboxyl groups and various derivatives thereof including, but not limited to, N-hydroxybenztriazole esters, acid halides, acyl imidazoles, thioesters, p-nitrophenyl esters, alkyl, alkenyl, alkynyl and aromatic esters;
(ii) hydroxyl groups, which can be converted to esters, ethers, aldehydes, etc.;
(iii) haloalkyl groups, wherein the halide can be displaced with a nucleophilic group such as, for example, an amine, a carboxylate anion, thiol anion, carbanion, or an alkoxide ion, thereby resulting in the covalent attachment of a new group at the site of the halogen atom;
(iv) dienophile groups, which are capable of participating in Diels-Alder reactions such as, for example, maleimido groups;
(v) aldehyde or ketone groups, such that subsequent derivatization is possible via formation of carbonyl derivatives such as, for example, imines, hydrazones, semicarbazones or oximes, or via such mechanisms as Grignard addition or alkyllithium addition;
(vi) alkenes, which can undergo, for example, cycloadditions, acylation, Michael addition, etc;
(vii) epoxides, which can react with, for example, amines and hydroxyl groups;
(ix) phosphoramidites and other standard functional groups useful in nucleic acid synthesis and
(x) any other functional group useful to form a covalent bond between the functionalized ligand and a molecular entity or a surface.

### Functional Groups with Non-specific Reactivities

In addition to the use of site-specific reactive moieties, the present invention contemplates the use of non-specific reactive groups to link a ligand to a targeting moiety. Non- specific groups include photoactivatable groups, for example.

Photoactivatable groups are ideally inert in the dark and are converted to reactive species in the presence of light. In one embodiment, photoactivatable groups are selected from precursors of nitrenes generated upon heating or photolysis of azides. Electron-deficient nitrenes are extremely reactive and can react with a variety of chemical bonds including N—H, O—H, C—H, and C═C. Although three types of azides (aryl, alkyl, and acyl derivatives) may be employed, arylazides are presently preferrred. The reactivity of arylazides upon photolysis is better with N—H and O—H than C—H bonds. Electron-deficient arylnitrenes rapidly ring-expand to form dehydroazepines, which tend to react with nucleophiles, rather than form C—H insertion products. The reactivity of arylazides can be increased by the presence of electron-withdrawing substituents such as nitro or hydroxyl groups in the ring. Such substituents push the absorption maximum of arylazides to longer wavelength. Unsubstituted arylazides have an absorption maximum in the range of 260-280 nm, while hydroxy and nitroarylazides absorb significant light beyond 305 nm. Therefore, hydroxy and nitroarylazides are most preferable since they allow to employ less harmful photolysis conditions for the affinity component than unsubstituted arylazides.

In another preferred embodiment, photoactivatable groups are selected from fluorinated arylazides. The photolysis products of fluorinated arylazides are arylnitrenes, all of which undergo the characteristic reactions of this group, including C—H bond insertion, with high efficiency (Keana et al., J. Org. Chem. 55: 3640-3647, 1990).

In another embodiment, photoactivatable groups are selected from benzophenone residues. Benzophenone reagents generally give higher crosslinking yields than arylazide reagents.

In another embodiment, photoactivatable groups are selected from diazo compounds, which form an electron-deficient carbene upon photolysis. These carbenes undergo a variety of reactions including insertion into C—H bonds, addition to double bonds (including aromatic systems), hydrogen attraction and coordination to nucleophilic centers to give carbon ions.

In still another embodiment, photoactivatable groups are selected from diazopyruvates. For example, the p-nitrophenyl ester of p-nitrophenyl diazopyruvate reacts with aliphatic amines to give diazopyruvic acid amides that undergo ultraviolet photolysis to form aldehydes. The photolyzed diazopyruvate-modified affinity component will react like formaldehyde or glutaraldehyde forming intraprotein crosslinks.

### Complexes

In one aspect, the invention provides a complex of a compound (ligand) disclosed herein with a metal ion.

Any of the combinations of compounds (ligands) disclosed herein and a metal ion disclosed herein are encompassed by this disclosure and specifically provided by the invention.

In some embodiments, the complex is luminescent.

In another aspect, the invention provides a complex of a compound (ligand) disclosed herein with an element, or ion thereof, from periods 4, 5, 6 and 7 and/or from groups 3, 4, 10, 11, 12, 13, 14, 15, 16.

### Metals

In some embodiments, the metal ion is an actinide. In some embodiments, the actinide is thorium (Th). In some embodiments, the metal ion is a lanthanide. In some embodiments, the lanthanide is terbium (Tb). In some embodiments, the lanthanide is europium (Eu). In some embodiments, the lanthanide is dysprosium (Dy). In some embodiments, the lanthanide is lutetium (Lu). In some embodiments, the lanthanide is gadolinium (Gd). In some embodiments the metal is yttrium (Y). In some embodiments, the metal is zirconium (Zr). In some embodiments, the metal ion is yttrium(III). In some embodiments, the metal ion is gallium (Ga). In some embodiments, the metal ion is europium(III). In some embodiments, the metal ion is terbium(III). In some embodiments, the metal ion is zirconium(IV). In some embodiments, the metal ion is thorium(IV). In some embodiments, the metal ion is selected from Th^{4′}, Zr⁴⁺, Eu³⁺, Dy³⁺, Tb³⁺, Lu³⁺, and Y³⁺. In some embodiments, the metal ion is Cu. In some embodiments, the metal ion is Ni. In some embodiments, the metal ion is Zn. In some embodiments, the metal ion is a radionuclide. In some embodiments, the metal ion is ²²⁷Th(IV). In some embodiments, the metal ion is ⁸⁹Zr(IV).

In some embodiments, the metal is ¹⁷⁷Lu. In some embodiments, the metal is ¹⁶⁶Ho. In some embodiments, the metal is ¹⁵³Sm. In some embodiments, the metal is ⁹⁰Y. In some embodiments, the metal is ⁸⁶Y. In some embodiments, the metal is ¹⁶⁶Dy. In some embodiments, the metal is ¹⁶⁵Dy. In some embodiments, the metal is ¹⁶⁹Er. In some embodiments, the metal is ¹⁷⁵yb. In some embodiments, the metal is ²²⁵AC. In some embodiments, the metal is ¹⁴⁹Tb. In some embodiments, the metal is ¹⁵³Gd. In some embodiments, the metal is ²³⁰U.

In some embodiments, the metal is ¹¹¹In. In some embodiments, the metal is ⁶⁷Ga. In some embodiments, the metal is ⁶⁷Cu. In some embodiments, the metal is 64 Cu. In some embodiments, the metal is ¹⁸⁶Re. In some embodiments, the metal is ¹⁸⁸Re. In some embodiments, the metal is ¹¹¹Ag. In some embodiments, the metal is ¹⁰⁹Pd. In some embodiments, the metal is ²¹²Pb. In some embodiments, the metal is ²⁰³Pb. In some embodiments, the metal is ²¹²Bi. In some embodiments, the metal is ²¹³Bi. In some embodiments, the metal is ^{195m}Pt. In some embodiments, the metal is ²⁰¹Tl. In some embodiments, the metal is ⁵⁵Co. In some embodiments, the metal is ^{99m}Tc.

### Radionuclides

The chelating moieties disclosed herein can be used to bind metal ions, in particular, a radionuclide. The term “radionuclide” or “radioisotope” refers to a radioactive isotope or element with an unstable nucleus that tends to undergo radioactive decay. Numerous decay modes are known in the art and include alpha decay, proton emission, neutron emission, double proton emission, spontaneous fission, cluster decay, β⁻ decay, positron emission (β⁺ decay), electron capture, bound state beta decay, double beta decay, double electron capture, electron capture with positron emission, double positron emission, isomeric transition and internal conversion.

Exemplary radionuclides include alpha-emitters, which emit alpha particles during decay. In some embodiments, a radionuclide is an emitter of a gamma ray or a particle selected from an alpha particle, an electron and a positron.

In some embodiments, the radionuclide is an actinide. In some embodiments, the radionuclide is a lanthanide. In some embodiments, the radionuclide is a 3+ ion. In some embodiments, the radionuclide is a 4⁺ ion. In some embodiments the radionuclide is a 2 +ion.

Of particular use in the complexes provided herein are radionuclides selected from isotopes of U, Pu, Fe, Cu, Sm, Gd, Tb, Dy, Ho, Er, Yb, Lu, Y, Th, Zr, In, Ga, Bi, Ra, At and Ac. In some embodiments, a radionuclide is selected form radium-223, thorium-227, astatine-211, bismuth-213, lutetium-177, and actinium-225. Other useful radioisotopes include bismuth-212, iodine-123, copper-64, copper-67, iridium-192, osmium-194, rhodium-105, samarium-153, and yttrium-88, yttrium-90, and yttrium-91. In exemplary embodiments, the radionuclide is thorium, particularly selected from thorium-227 and thorium-232. In some embodiments, thorium-226 is excluded. In some embodiments, U is excluded. In some embodiments, uranium-230 is excluded. That is, in some embodiments, a radionuclide is not U, or a radionuclide is not uranium-230 or a radionuclide is not thorium-226.

Exemplary embodiments of the invention comprise the metal copper, Cu(II), and radioactive isotopes thereof, such as Cu-64 and Cu-67.

Exemplary embodiments of the invention comprise the metals zinc (Zn), Zn(II), lead (Pb), and Pb(II), and radioactive isotopes thereof. Further embodiments include other metals including gallium, actinium, cobalt, manganese, iron, indium, and other lanthanides, ions such as Ga(III), Ac(III), Co(II), Mn(III), Fe(II), In(III) and radioactive active isotopes of these metals.

Exemplary radionuclides include, without limitation, Cu(II), Zn(II), Pb(II), Ga(III), ⁶⁴Cu(II), ⁶⁷Cu(II), ²¹²Pb(II), ⁶⁷Ga(III), ⁶⁸Ga(III), ⁶⁴Cu, ⁶⁷Cu, ²¹²Pb, ⁶⁷Ga, ⁶⁸Ga, Pt(II), Pd(II), Ni(II), and Cd(II).

### 3. Uses

The ligands and complexes disclosed herein can be used in a wide variety of therapeutic and diagnostic settings.

In one aspect, the invention provides a method of treating a disease in an animal comprising administering a complex disclosed herein to the animal, whereby the disease is ameliorated or eliminated.

In one aspect, the invention provides a method of diagnosing a disease in an animal comprising (a) administering a complex disclosed herein to the animal and (b) detecting the presence or absence of a signal emitted by the complex. In some embodiments, the detecting step comprises obtaining an image based on the signal.

In some embodiments, the disease is cancer.

In some embodiments, the complex comprises a linker to a targeting moiety and the method further comprises localizing the complex to a targeting site in the animal by binding the targeting moiety to the targeting site.

The compounds disclosed herein are particularly well suited for the preparation of stable, pre-labeled antibodies for use in the diagnosis and treatment of cancer and other diseases. For example, antibodies expressing affinity for specific tumors or tumor-associated antigens are labeled with a diagnostic radionuclide-complexed chelate, and the labeled antibodies can be further stabilized through lyophilization. Where a chelate is used, it generally is covalently attached to the antibody. The antibodies used can be polyclonal or monoclonal, and the radionuclide-labeled antibodies can be prepared according to methods known in the art. The method of preparation will depend upon the type of radionuclide and antibody used. A stable, lyophilized, radiolabeled antibody can be reconstituted with suitable diluent at the time of intended use, thus greatly simplifying the on site preparation process. The methods of the invention can be applied to stabilize many types of pre-labeled antibodies, including, but not limited to, polyclonal and monoclonal antibodies to tumors associated with melanoma, colon cancer, breast cancer, prostate cancer, etc. Such antibodies are known in the art and are readily available.

### EXAMPLES

### Introduction

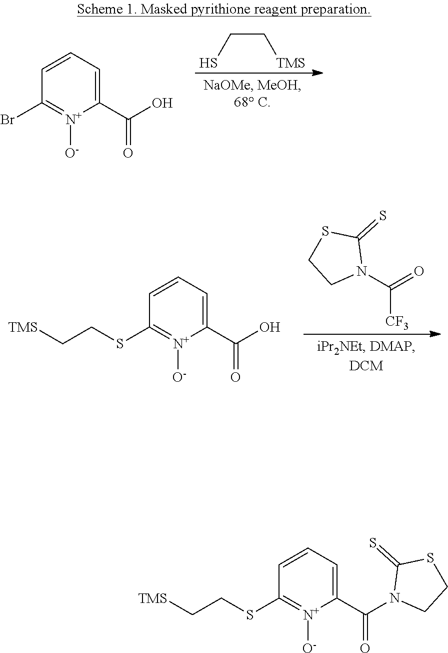 [SMILES: O=C(O)c1cccc(Br)[n+]1[O-] ; C[Si](C)(C)CCS ; C[Si](C)(C)CCSc1cccc(C(=O)O)[n+]1[O-] ; O=C(N1CCSC1=S)C(F)(F)F ; C[Si](C)(C)CCSc1cccc(C(=O)N2CCSC2=S)[n+]1[O-]]

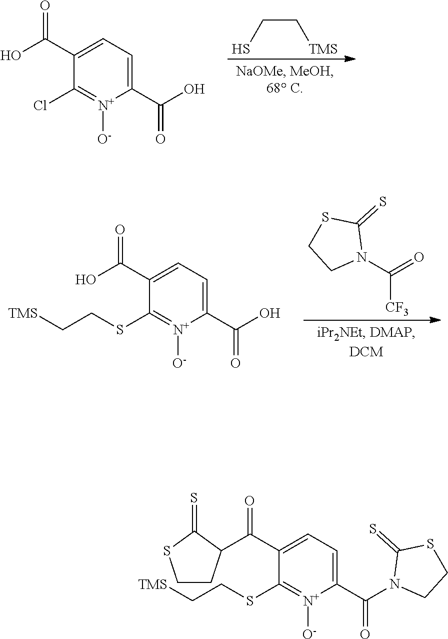 [SMILES: O=C(O)c1ccc(C(=O)O)[n+]([O-])c1Cl ; C[Si](C)(C)CCS ; C[Si](C)(C)CCSc1c(C(=O)O)ccc(C(=O)O)[n+]1[O-] ; O=C(N1CCSC1=S)C(F)(F)F ; C[Si](C)(C)CCSc1c(C(=O)C2CCSC2=S)ccc(C(=O)N2CCSC2=S)[n+]1[O-]]

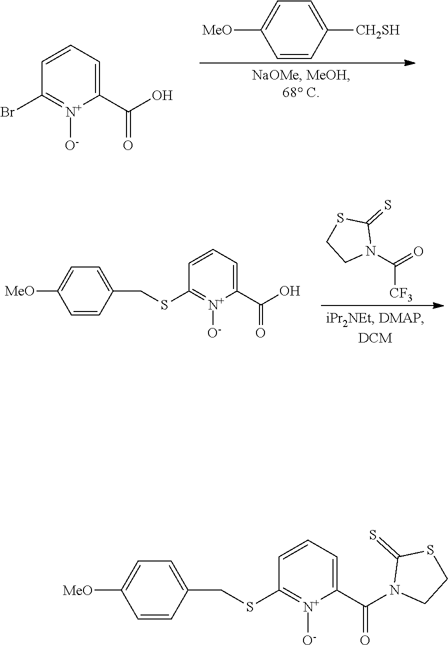

Since metal ions such as copper(II) typically form four-coordinate complexes, initial work has focused on the preparation of chelators containing two pyrithione groups. Model compounds containing only one pyrithione group have also been prepared. Initial work has involved the preparation of an open chain bis-pyrithione bi-functional chelator similar to those chelators reported at University of California, Berkeley (e.g., Abu-Dari, K., et al. references cited above) but also containing functionality for forming derivatives with, for example, a site directing molecule such as an antibody. In the example shown in FIG. 2, the bis-pyrithione chelator is treated with copper acetate to form the copper chelate, which is activated by forming the NHS ester for conjugation with a ligand for prostate specific membrane antigen (PSMA).

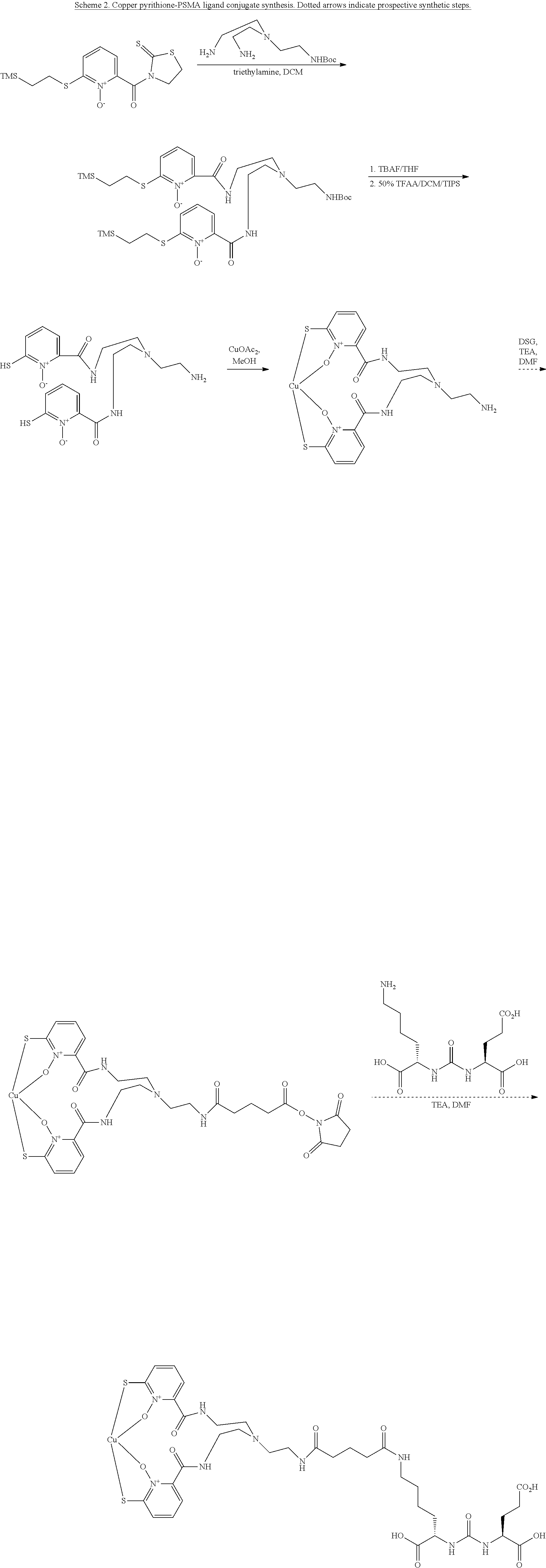

The copper in this example serves as a protective group to prevent reaction of the pyrithione moieties with the NHS ester reagent or product. This is appropriate if the copper complex is the final product of synthesis. The described conjugate might act as an ionophore to deliver copper site-specifically to prostate cancer and thereby inhibit its growth. Alternatively, another metal ion that is less well bound by the chelator, such as magnesium or zinc, may permit the synthesis of an NHS ester or isothiocyanate intermediate. The magnesium or zinc may be displaced from the chelate in the final conjugate by other metal ions, e.g., radioactive copper isotopes, for use in radioisotope therapy or imaging applications.

Preparation of a mono-macrocyclic bis-pyrithione bi-functional chelator is shown in Scheme 3. Macrocycle formation increases the stability of the metal ion chelate, which might be essential for in vivo applications. However, due to the two possible orientations of the di-carboxamide moiety in this macrocycle, multiple regioisomers can be present in the final product. Regioisomeric products can be made selectively. One approach, similar to that employed in preparation of 1,2-HOPO macrocyclic systems, involves isolation and elaboration of an intermediate monoester. However, it has been discovered that the two activated carbonyl groups present in the di-thiazolide intermediate display sufficiently different reactivity so as to allow selective preparation of individual isomeric products. Alternative macrocyclic systems containing pyrithione coordinating groups are also shown in Scheme 4. Finally, the preparation of a related mono-macrocyclic 1,2-HOPO system is contemplated (Scheme 5). This system may be used for comparison with the corresponding pyrithione containing analogue, to determine, for example, which chelator displays more attractive properties for copper-64 chelation for PET imaging and copper-67 for therapy. Synthetic examples of work conducted so far or prospective examples follow. Please note that several tautomeric forms may be used to depict pyrithione systems, Cf, e.g.: Scheme 6. Particular pyrithione tautomers are drawn for convenience and are not intended to depict the most prevalent form present in solution.

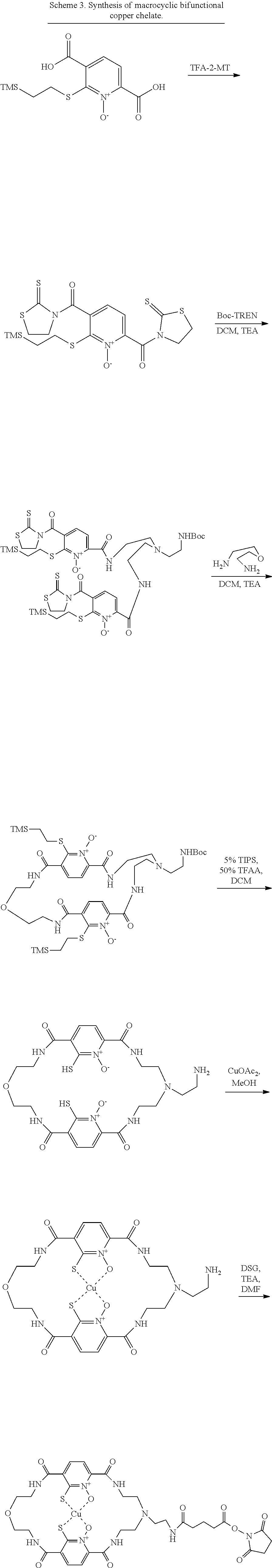

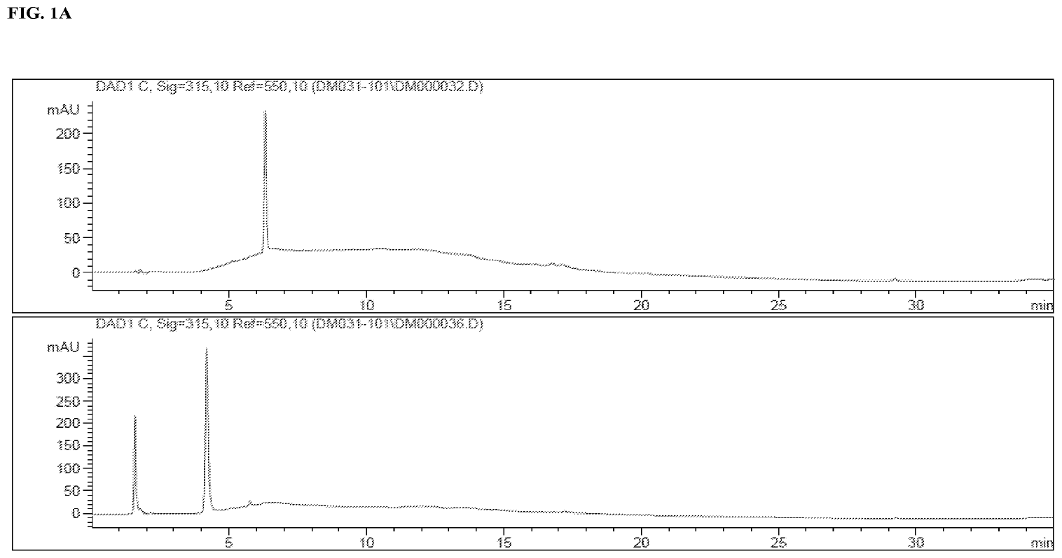

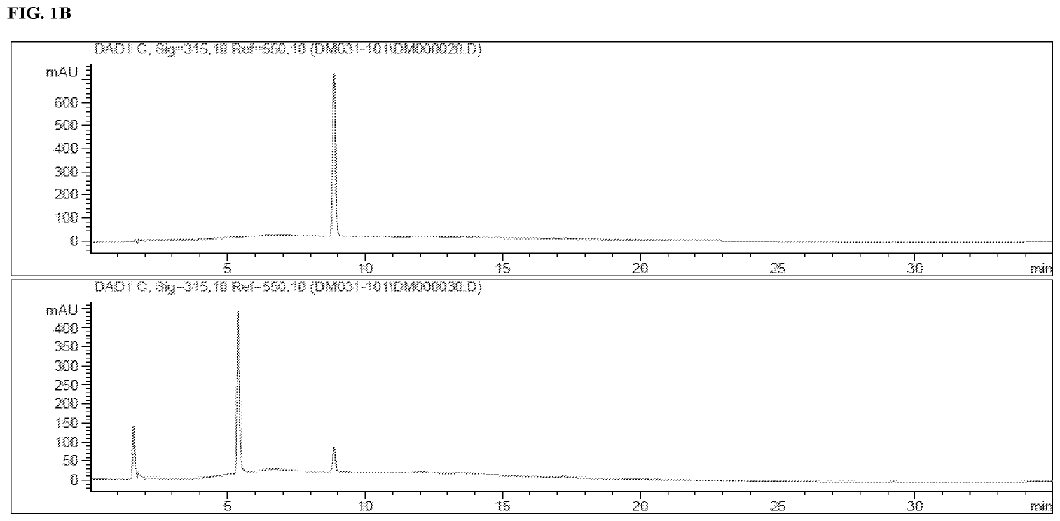

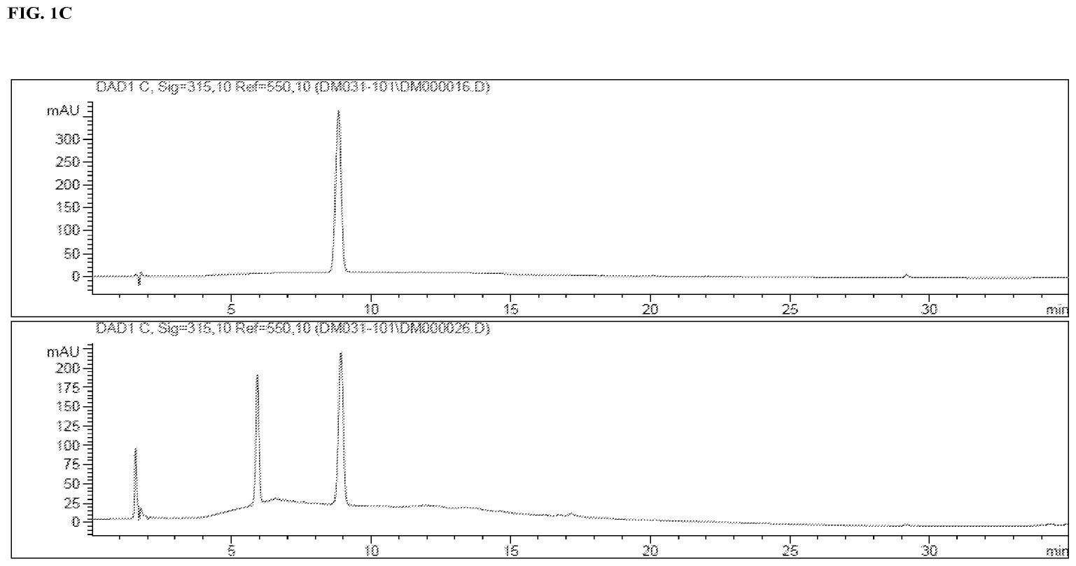

The structures provided in FIGS. 1-6 are not limited to the metal-chelate combination, additional metals that may also have useful binding properties within the provided structures.

Preferred embodiments of the invention comprise the metal copper, Cu(II), and radioactive isotopes thereof, such as Cu-64 and Cu-67.

Other embodiments include zinc and lead, Zn(II), Pb(II), and radioactive isotopes thereof.

Further embodiments include other metals including gallium, actinium, cobalt, manganese, iron, indium, and other lanthanides, ions such as Ga(III), Ac(III), Co(II), Mn(III), Fe(II), In(III) and radioactive active isotopes of these metals.

Certain structures in FIG. 4 have higher stability, faster chelation, or other beneficial characteristics for some of the metals above, compared to others. Thus this disclosure provides a wide range of chelate structures based on representative examples of FIGS. 1-6, that can chelate a wide range of metals with novel and useful characteristics including treatment and diagnosis of diseases.

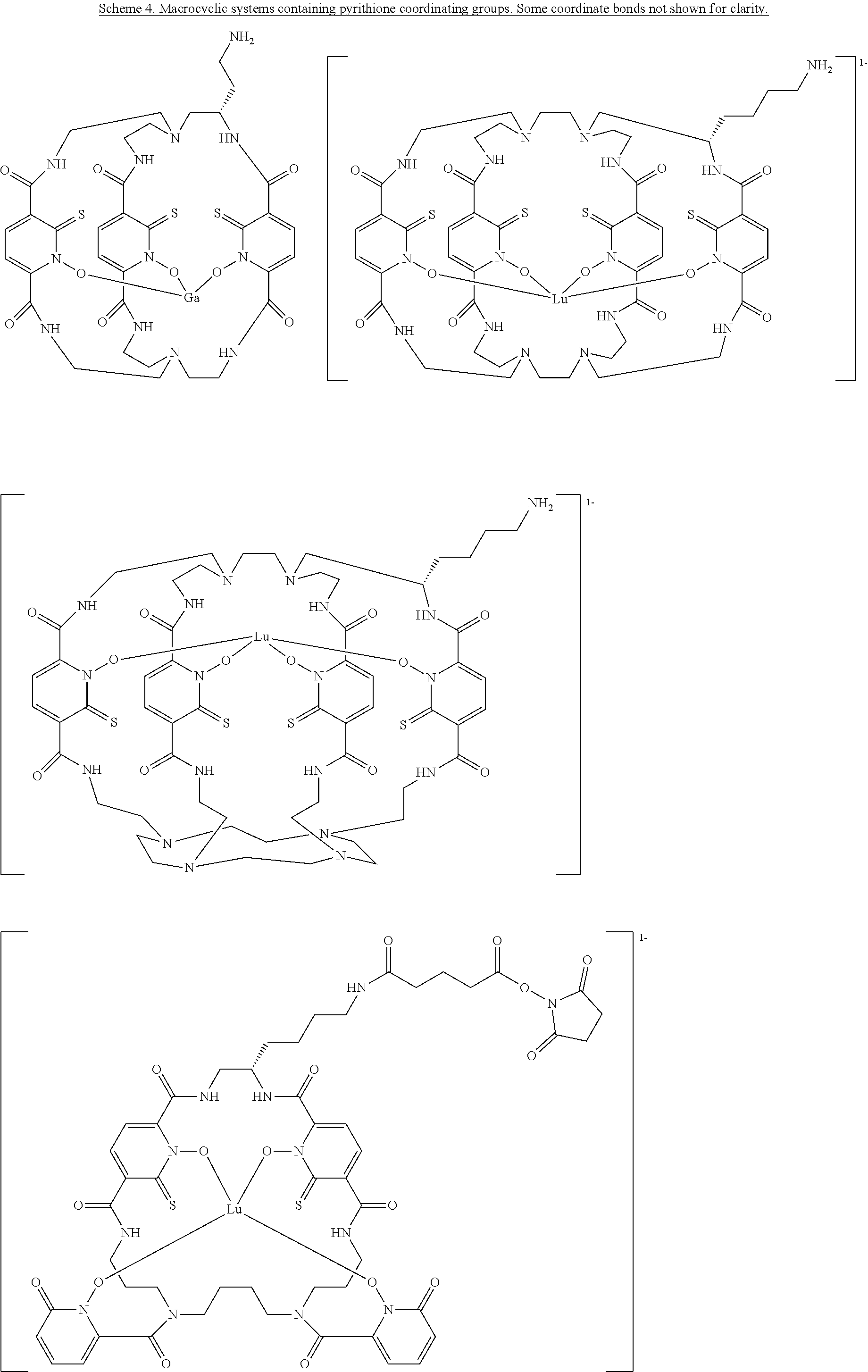

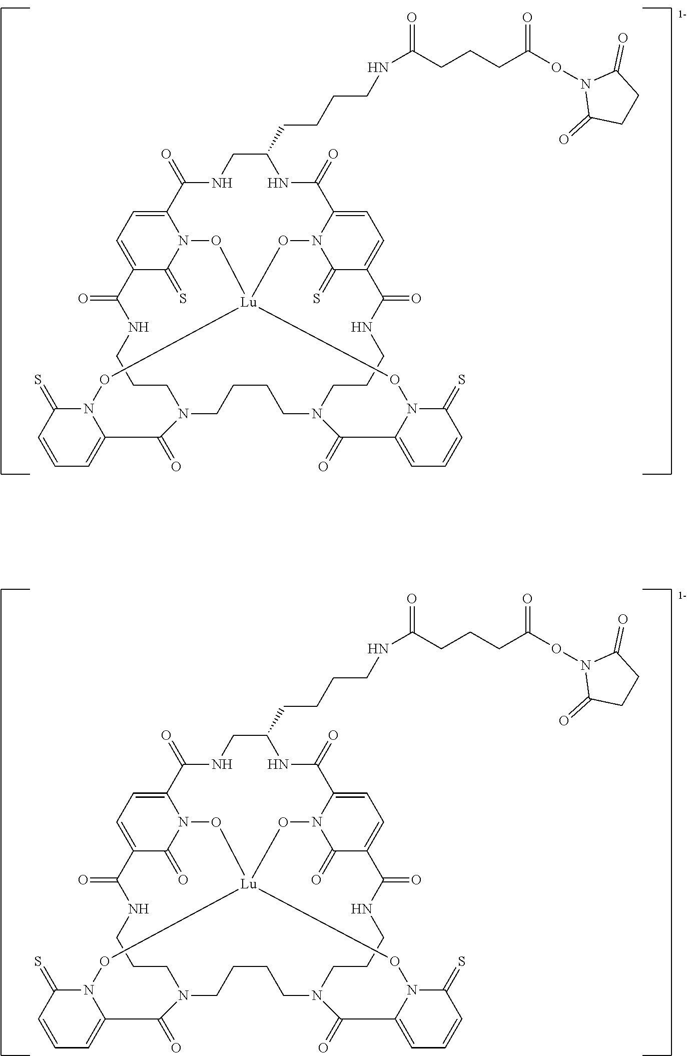

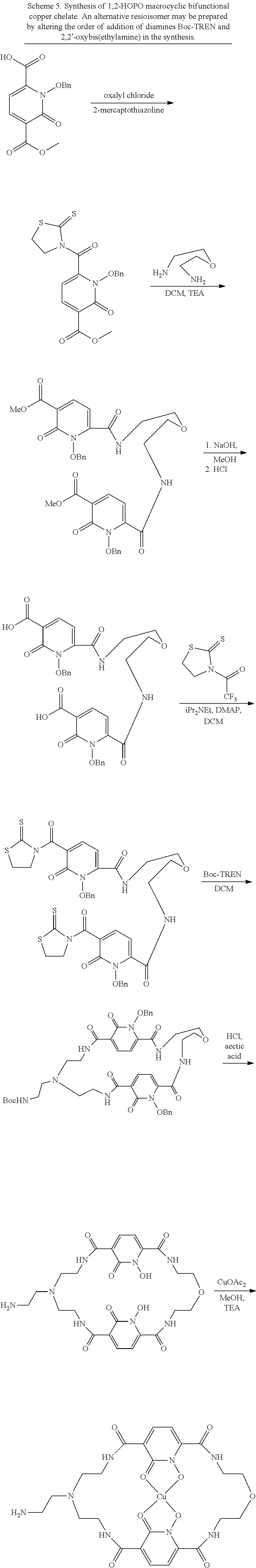

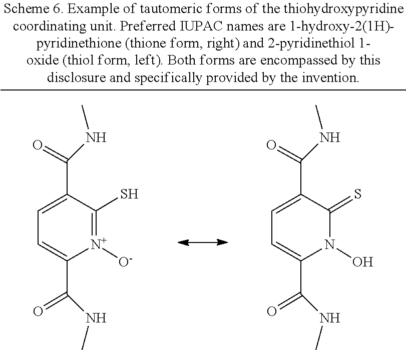 [SMILES: CNC(=O)c1ccc(C(=O)NC)[n+]([O-])c1S ; CNC(=O)c1ccc(C(=O)NC)n(O)c1=S]

### EXAMPLES

### Example 1

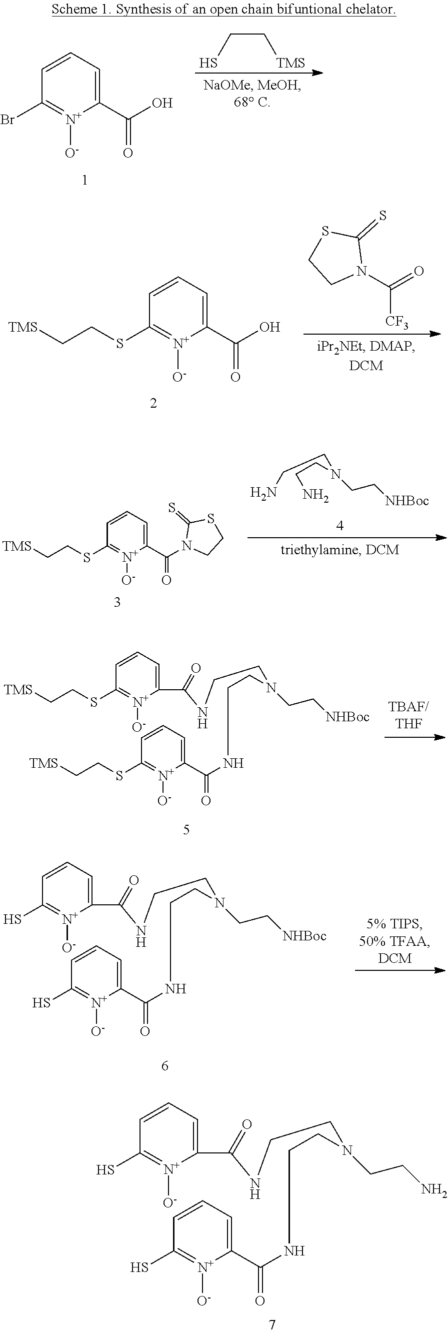

2-Bromo-6-carboxypyridine 1-oxide 1 was prepared as previously described (Scarrow, R. C.; Riley, P. E.; Abu-Dari, K.; White, D. L.; Raymond, K. N. Inorg. Chem. 1985, 24, 954.). Reagents were purchased from Sigma-Aldrich Chemicals unless noted otherwise. 2,2,2-Trifluoro-1-(2-thioxothiazolidin-3-yl)ethan-1-one was prepared according to literature protocol (Nagao, Y., et al., J. Organometal. Chem. 2000, 611, 172-177.). tert-Butyl (2-(bis(2-aminoethyl)amino)ethyl)carbamate 4 was prepared as described (Pham, T. A., Xu, J., Raymond, K. N. J. Am. Chem. Soc. 2014, 136, 25, 9106-9115.) or purchased from Aldrich.

2-Carboxy-6-((2-(trimethylsilyl)ethyl)thio)pyridine 1-oxide 2. 2-Bromo-6-carboxypyridine 1-oxide 1 (500 mg, 2.29 mmol) was dried overnight in a 100 mL flask equipped with a condenser and stir bar. Methanol (20 mL) was added to form a solution, and 2-(trimethylsilyl)ethanethiol (459 μL, 2.86 mmol) was added via syringe under an inert atmosphere. Sodium methoxide (30% by weight in methanol, 1.24 mL, 6.87 mmol) was added, and the solution was heated at 68° C. for 4.5 hr. Solvents were removed under reduced pressure, and the residue was dried overnight in vacuo. The residue was dissolved in 20 mL water, and 1N HCl (ca. 10 mL) was added to form a precipitate. The precipitate was filtered, washed with water (ca. 10 mL) and dried in vacuo to provide compound 2 (576 mg, 92.7%) as a dusty powder. HRMS-pESI: Calc. for C₁₁H₁₆NO₃SSi (M−H)⁻, 270.0626; found, 270.0626.

2-(2-Thioxothiazolidine-3-carbonyl)-6-((2-(trimethylsilyl)ethyl)thio)pyridine 1-oxide 3. Compound 2 (500 mg, 1.84 mmol) and 4-(dimethylamino)pyridine (90 mg, 734 μmol) were dried together overnight in a 100 mL flask. The solids were dissolved in dichloromethane (anhydrous, 20 mL), N, N-diispropylethylamine (963 μL, 5.53 mmol) was added, and then 2,2,2-trifluoro-1-(2-thioxothiazolidin-3-yl)ethan-1-one (630 mg, ca. 396 μL, 2.93 mmol) was added using a pipette. The solution, which gradually turns from orange to yellow color, was allowed to stir for 4.5 hr. Solvents were removed and the residue was dried overnight. The residue was purified using silica gel chromatography, using 2% isopropyl alcohol in dichloromethane to elute compound 3, which formed a yellow solid upon removal of solvent and that was dried in vacuo (582 mg, 84.9%). FTMS+pESI: Calc. for C₁₄H₂₁N₂O₂S₃Si (M+H)⁺, 373.0529; found, 373.0528.

2-((2-((2-((Tert-butoxycarbonyl)amino)ethyl)(2-(1-oxido-6-((2-(trimethylsilyl)ethyl)thio)pyridine-2-carboxamido)ethyl)amino)ethyl)carbamoyl)-6-((2- (trimethylsilyl)ethyl)thio)pyridine 1-oxide 5. tert-Butyl (2-(bis(2-aminoethyl)amino)ethyl)carbamate 4 (146 mg, 593 μmol) was dissolved in anhydrous dichloromethane (15 mL) and triethylamine (248 μL, 1.779 mmol). Compound 3 (486 mg, 1.304 mmol) was added all at once, and the resulting solution was stirred under inert atmosphere for 23 hr. Solvents were removed under reduced pressure, and the residue was purified using silica gel chromatography, using 2-3.5% methanol in dichloromethane supplemented with 0.1% triethylamine to elute compound 5, which formed a yellow solid that was dried in vacuo (389 mg, 87.1%). FTMS+pESI: Calc. for C₃₃H₅₇N₆O₆S₂Si₂ (M+H)⁺, 753.3314; found, 753.3308.

2-((2-((2-((tert-butoxycarbonyl)amino)ethyl)(2-(6-mercapto-1-oxidopyridine-2-carboxamido)ethyl)amino)ethyl)carbamoyl)-6-mercaptopyridine 1-oxide 6. Compound 5 (100 mg, 133 μmol) was dried overnight in a 10 mL flask. Anhydrous tetrahydrofuran (0.5 mL) was added to form a solution. Tetra-n-butyl ammonium fluoride (1M solution in tetrahydrofuran, 399 μL, 399 μmol) was added, and the resulting red solution was stirred under inert atmosphere for 5 hr. A solution of 1M ammonium acetate, pH 4.4 (20 mL) was added, and the resulting yellow solution was applied to a reverse phase column (Sep-pak tC18, 20 cc/5 g, Waters Corp.). The column was washed with 0.1M ammonium acetate, pH 4.4 (50 mL), with 2.5% acetonitrile in 0.1M ammonium acetate (20 mL), with 5% acetonitrile in 0.1M ammonium acetate (20 mL), with 10% acetonitrile in 0.1M ammonium acetate (40 mL), and with water (45 mL). The yellow product was eluted with acetonitrile (15 mL) and the compound was dried under reduced pressure to provide crude compound 6 (107 mg, 146%) as a yellow solid. Crude compound 6 was used without further purification.

6,6′-(((((2-aminoethyl)azanediyl)bis(ethane-2,1-diyl))bis(azanediyl))bis(carbonyl))bis(2-mercaptopyridine 1-oxide) 7. A mixture of 5% triisopropylsilane (AK Scientific), 50% trifluoroacetic acid and 45% dichloromethane (200 μL) was added to crude compound 6 (16 mg, 29 μmol) in a 10 mL flask and the solution was stirred for 1 hr. Solvents were removed in vacuo to provide crude compound 7.

### Example 2

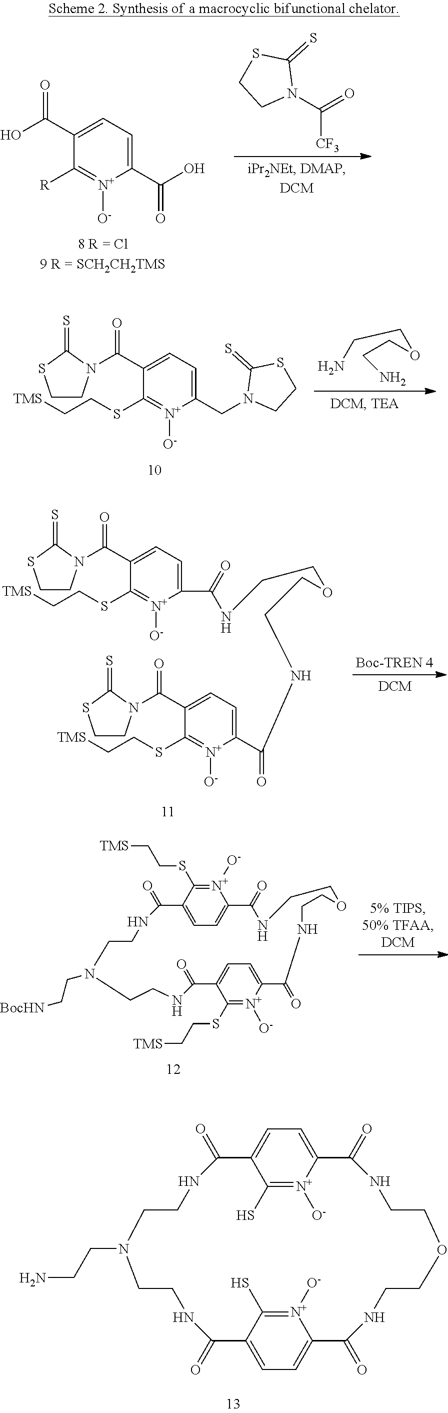

3,6-Dicarboxy-2-chloropyridine 1-oxide 8 was prepared as described (WO/2019/173639).

3,6-Dicarboxy-2-((2-(trimethylsilyl)ethyl)thio)pyridine 1-oxide 9. 3,6-Dicarboxy-2-chloropyridine 1-oxide 8 (500 mg, 2.30 mmol) was dried overnight in a 100 mL flask equipped with a condenser and stir bar. Methanol (20 mL) was added to form a solution, and 2-(trimethylsilyl)ethanethiol (460 μL, 2.87 mmol) was added via syringe under an inert atmosphere. Sodium methoxide (30% by weight in methanol, 1.66 mL, 9.2 mmol) was added, and the solution was heated at 68° C. for 3.5 hr. Solvents were removed under reduced pressure, and the residue was dried overnight in vacuo. The residue was dissolved in 20 mL water, and 1N HCl (ca. 10 mL) was added to form a precipitate. The precipitate was filtered, washed with water (ca. 20 mL) and dried in vacuo to provide compound 9 (480 mg, 66.2%) as a dusty powder. HRMS-pESI: Calc. for C₁₂H₁₆NO₅SSi (M−H)⁻, 314.0524; found, 314.0524.

3,6-Bis(2-thioxothiazolidine-3-carbonyl)-2-((2-(trimethylsilyl)ethyl)thio)pyridine 1-oxide 10. Compound 9 (300 mg, 951 μmol) and 4-(dimethylamino)pyridine (46 mg, 376 μmol) were dried together overnight in a 50 mL flask. The solids were dissolved in dichloromethane (anhydrous, 12 mL), N,N-diispropylethylamine (994 μL, 5.71 mmol) was added, and then 2,2,2-trifluoro-1-(2-thioxothiazolidin-3-yl)ethan-1-one (651 mg, ca. 409 μL, 3.02 mmol) was added using a pipette. The solution, which gradually turns from orange to yellow color, was allowed to stir for 6.75 hr. Solvents were removed and the residue was dried overnight. The residue was purified using silica gel chromatography, using 1% isopropyl alcohol in dichloromethane to elute crude compound 10, which formed a yellow solid upon removal of solvent and that was dried in vacuo (473 mg, 96.1%). The compound was further purified by dissolving the residue in dichloromethane and forming a precipitate by adding tert-butyl methyl ether to provide purified 10 (390 mg, 79.2%). FTMS+pESI: Calc. for C₁₈H₂₄H₃O₃S₅Si (M+H)⁺, 518.0185; found, 518.0187.

6,6′-(((Oxybis(ethane-2,1-diyl))bis(azanediyl))bis(carbonyl))bis(3-(2-thioxothiazolidine-3-carbonyl)-2-((2-(trimethylsilyl)ethyl)thio)pyridine 1-oxide) 11. Compound 10 (1.500 g, 2.90 mmol) was dissolved in anhydrous dichloromethane (60 mL) and cooled on an ice bath. 2,2′-Oxybis(ethylamine), dihydrochloride (Tokyo Chemicals, 231 mg, 1.30 mmol) then triethylamine (545 μL, 3.91 mmol) were added and the solution was allowed to slowly warm to ambient temperature overnight. After 20 hours, solvents were removed and the residue was dried overnight. The residue was purified using silica gel chromatography, using 1-3.5% isopropyl alcohol in dichloromethane to elute compound 11 that formed a yellow solid upon removal of solvent and that was dried in vacuo (1.094 g, 93.4%). FTMS+pESI: Calc. for C₃₄H₄₉N₆O₇S₆Si₂ (M+H)⁺, 901.1520; found, 901.1513.

16-(2-((Tert-butoxycarbonyl)amino)ethyl)-2,10,12,20-tetraoxo-1⁶,11⁶-bis((2-(trimethylsilyl)ethyl)thio)-6-oxa-3,9,13,16,19-pentaaza-1,11(2,5)-dipyridin-1- iumacycloicosaphane 1¹,11¹-dioxide 12. Compound 11 (511 mg, 567 μmol) was dissolved in anhydrous dichloromethane and anhydrous acetonitrile (1:1 mixture, 27 mL) and transferred to a mL Hamilton syringe. tert-Butyl (2-(bis(2-aminoethyl)amino)ethyl)carbamate 4 (140 mg, 567 μmol) was dissolved in anhydrous acetonitrile (27 mL) and transferred to another syringe. The two reactants were added to a stirring pot of dichloromethane (500 mL) at a rate of 0.18-0.50 mL/hr over 4 days using two syringe pumps. An additional amount of compound 4 (47.7 mg, 194 μmol) was dissolved in 11 mL acetonitrile and added at a rate of 0.50 mL hour over an additional day. Solvents were removed and the residue was dried overnight. The residue was purified using silica gel chromatography, using 0.1% triethylamine, 2-5% methanol in dichloromethane to elute compound 11 as an off white solid that was dried in vacuo (441 mg, FTMS+pESI: Calc. for C₃₉H₆₅N₈O₉S₂Si₂ (M+H)⁺, 909.3849; found, 909.3827.

16-(2-Aminoethyl)-1⁶,11⁶-dimercapto-2,10,12,20-tetraoxo-6-oxa-3,9,13,16,19-pentaaza-1,11(2,5)-dipyridin-1-iumacycloicosaphane 1¹,11¹-dioxide 13. Compound 12 (100 mg, 110 μmol) was dissolved in a mixture of 5% triisopropylsilane, 50% trifluoroacetic acid in dichloromethane (2.0 mL). The resulting solution was stirred under a nitrogen atmosphere for 9 hours. Solvents were removed under reduced pressure and the product was dried in vacuo overnight. The residue was dissolved in methanol (1 mL) and transferred to 3 o-ring type 2 mL microcentrifuge tubes. Diethyl ether (1.5 mL per tube) was added to form a precipitate. The tubes were centrifuged at 13,000 rpm for 3 minutes, the supernatant removed, and the pellets were washed with diethyl ether (1.5 mL per tube). The tubes were centrifuged at 13,000 rpm for 3 minutes, the supernatant removed, and the pellets were air dried 10 minutes then dried in vacuo to provide compound 13 as the trifluoroacetate salt (64.2 mg, 87.8%). FTMS+pESI: Calc. for C₂₄H₃₃N₈O₇S₂ (M+H)⁺, 609.1908; found, 609.1907.

### Example 3

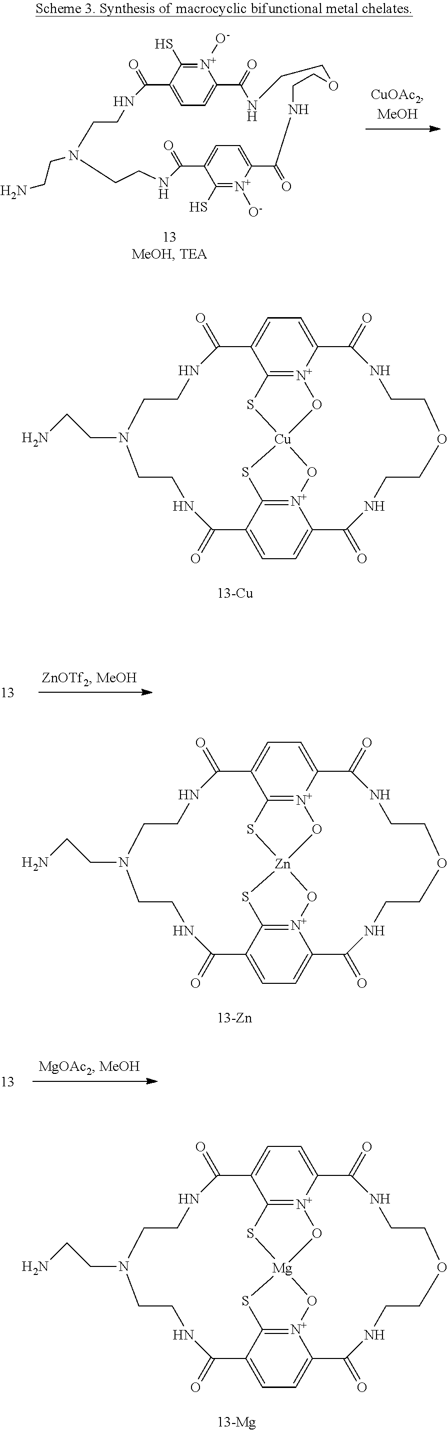

16-(2-Aminoethyl)-1⁶,11⁶-dimercapto-2,10,12,20-tetraoxo-6-oxa-3,9,13,16,19-pentaaza-1,11(2,5)-dipyridin-1-iumacycloicosaphane 1¹,11¹-dioxide 13 (2.32 mg, 2.80 μmol) was dissolved in methanol (Aldrich Trace Select, 130.9 μL) and triethylamine (7.7 μL). Copper(II) acetate monohydrate (Oakwood Chemicals, 2.23 mg) was dissolved in methanol (Aldrich Trace Select, 1228 μL). Magnesium acetate (ChemImpex Chemicals, 0.97 mg) was dissolved in methanol (Aldrich Trace Select, 452.3 μL). Zinc triflate (Aldrich Chemicals, 1.65 mg) was dissolved in methanol (Aldrich Trace Select, 453.9 μL). The above solution of compound 13 (40 μL) was added to an aliquot of each stock metal ion solution (100 μL) in 2 mL microcentrifuge tubes and mixed by vortexing for 5 minutes. A color change (less yellow) was noted in the reaction containing copper, and a precipitate was formed in both copper and zinc containing reactions. Isopropyl alcohol (500 μL/tube) was added to the copper and zinc containing reactions. The tubes were centrifuged for 3 minutes at 13,000 rpm to form pellets which were washed with isopropyl alcohol (500 μL) and dried in vacuo. Diethyl ether (500 μL) was added to the magnesium containing reaction to form a precipitate. The tube was centrifuged for 3 minutes at 13,000 rpm to form a pellet which was dissolved in isopropyl alcohol (500 μL). Diethyl ether (1 mL) was added to form a precipitate that was isolated by centrifugation and dried in vacuo. Copper complex of compound 13: FTMS+pESI: Calc. for C₂₄H₃₁N₈O₇CuS₂ (M+H)⁺, 670.1048; found, 670.1045. Zinc complex of compound 13: FTMS+pESI: Calc. for C₂₄H₃₁N8O₇S₂Zn (M+H)⁺, 671.1043; found, 671.1040. Magnesium complex of compound 13: FTMS+pESI: Calc. for C₂₄H₃₁N₈O₇MgS₂ (M+H)⁺, 631.1602; found, 631.1604.

### Example 4

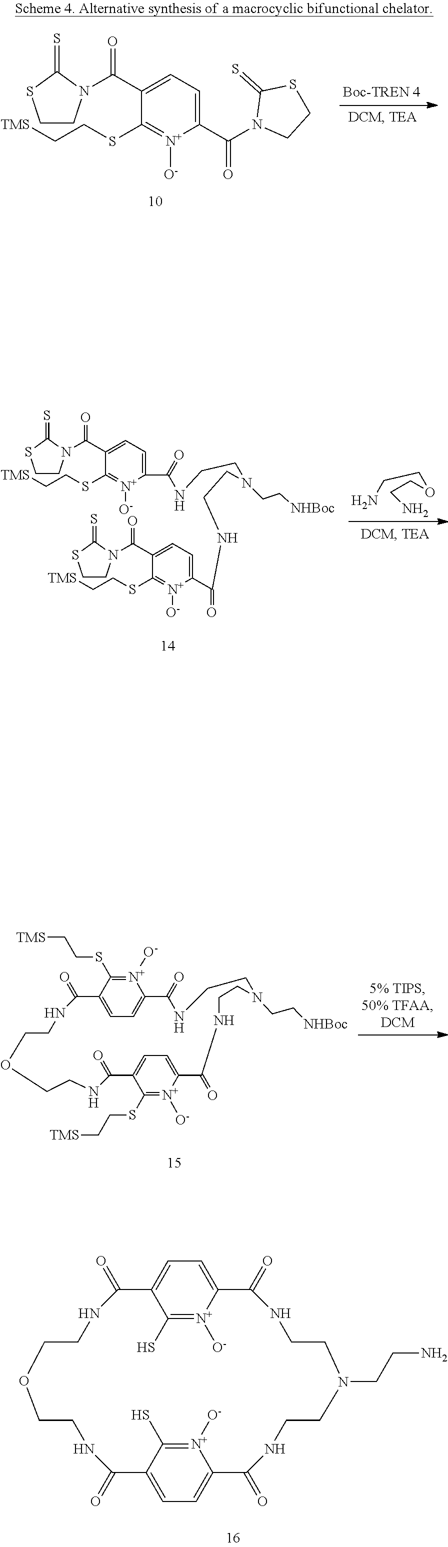 [SMILES: C[Si](C)(C)CCSc1c(C(=O)N2CCSC2=S)ccc(C(=O)N2CCSC2=S)[n+]1[O-] ; CC(C)(C)OC(=O)NCCN(CCNC(=O)c1ccc(C(=O)N2CCSC2=S)c(SCC[Si](C)(C)C)[n+]1[O-])CCNC(=O)c1ccc(C(=O)N2CCSC2=S)c(SCC[Si](C)(C)C)[n+]1[O-] ; NCCOCCN ; CC(C)(C)OC(=O)NCCN1CCNC(=O)c2ccc(c(SCC[Si](C)(C)C)[n+]2[O-])C(=O)NCCOCCNC(=O)c2ccc([n+]([O-])c2SCC[Si](C)(C)C)C(=O)NCC1 ; NCCN1CCNC(=O)c2ccc(c(S)[n+]2[O-])C(=O)NCCOCCNC(=O)c2ccc([n+]([O-])c2S)C(=O)NCC1]

The synthesis is conducted as in Example 2 above, except that the diamine reagents are added in opposite order.

### Example 5

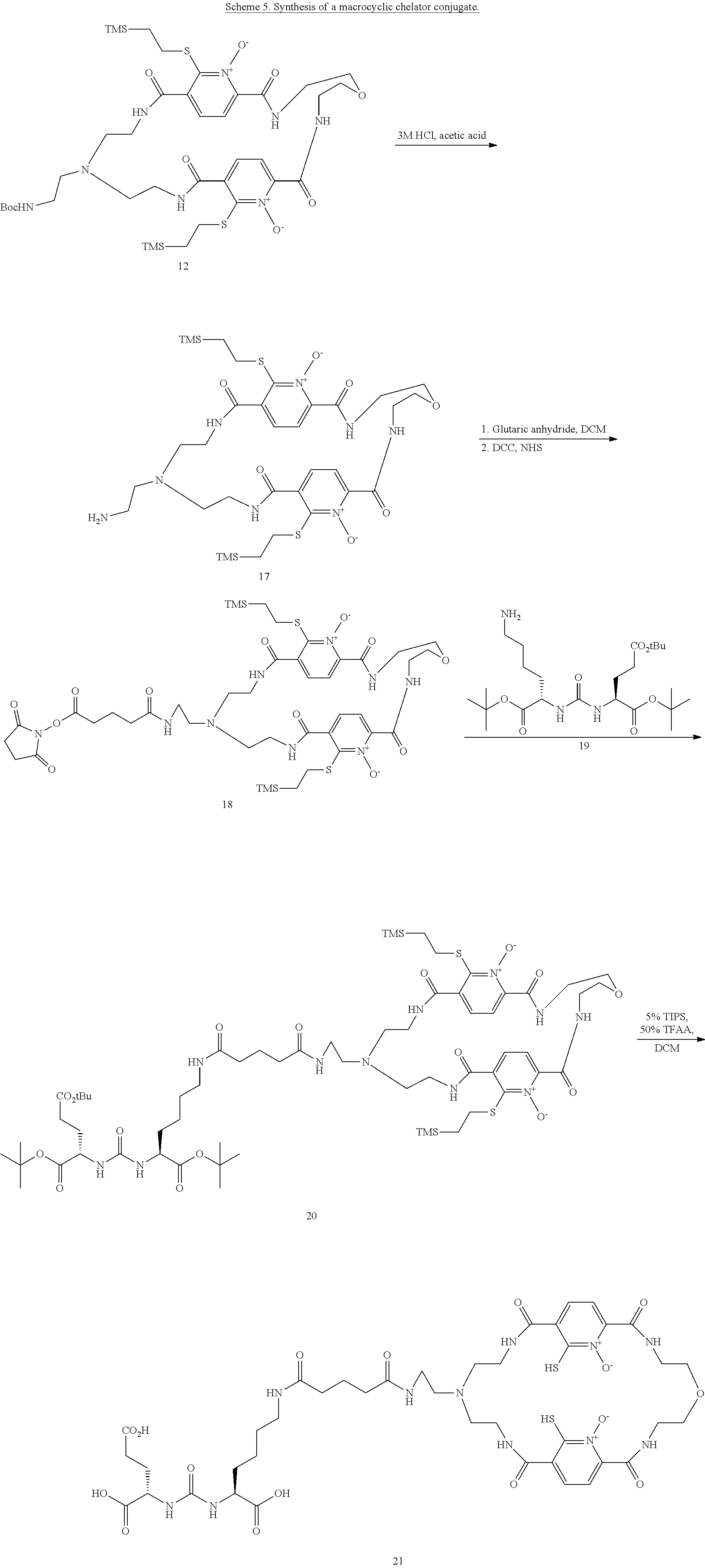

16-(2-Aminoethyl)-2,10,12,20-tetraoxo-1⁶,11⁶-bis((2-(trimethylsilyl)ethyl)thio)-6-oxa-3,9,13,16,19-pentaaza-1,11(2,5)-dipyridin-1-iumacycloicosaphane 1¹,11¹-dioxide 17. Compound 12 (50 mg, 55 μmol) was dissolved in acetic acid (0.5 mL). Hydrochloric acid (6N, mL) was added, and the reaction was stirred under a nitrogen atmosphere for 75 minutes. Hydrochloric acid was removed using a stream of nitrogen gas, and solvents were removed in vacuo. The residue was dissolved in methanol (1 mL) and transferred to 4 2-mL microcentrifuge tubes. Ether (1.7 mL per tube) was added to form a precipitate. The tubes were centrifuged for 1 minute at 12,000 rpm, the supernatant removed, and the precipitate was washed with ether (1.5 mL per tube). The tubes were centrifuged, the supernatant decanted and the precipitate allowed to dry in air briefly, then dried overnight in vacuo to provide the hydrochloride salt of compound 17 as an off white foam. FTMS+pESI: Calc. for C₃₄H₅₇N₈O₇S₂Si₂ (M+H)⁺, 809.3325; found, 809.3324.

### Example 6

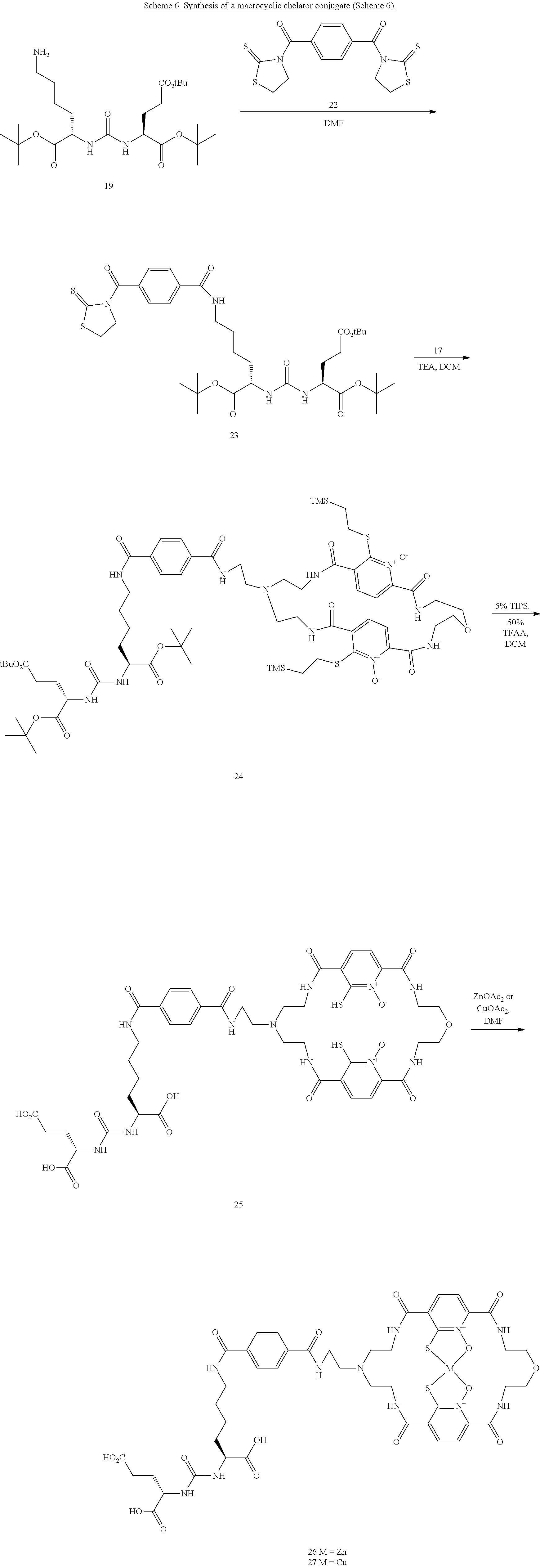

1,4-Phenylenebis((2-thioxothiazolidin-3-yl)methanone) 22. Terephthalic acid (2.00 g, 12.0 mmol) and 4-dimethylaminopyridine (294 mg, 2.41 mmol) were dried overnight in vacuo. The solids were dissolved in dichloromethane (anhydrous, 60 mL), N,N-diispropylethylamine (5.24 mL, 30.1 mmol) was added, and then 2,2,2-trifluoro-1-(2-thioxothiazolidin-3-yl)ethan-1-one (4.77 g, ca. 3.0 mL, 22.2 mmol) was added using a pipette. After 4 hr, additional dichloromethane (60 mL) and N,N-diispropylethylamine (ca. 5 mL, 28.7 mmol) were added to the suspension, which was allowed to stir overnight. Solvents were removed under reduced pressure, and the residue was suspended in dichloromethane (50 mL) and filtered using a medium fritted funnel. The solids were washed with dichloromethane (3×10 mL) and then dried in vacuo to provide compound 22 (2.477 g, 56%).

Di-tert-butyl (((S)-1-(tert-butoxy)-1-oxo-6-(4-(2-thioxothiazolidine-3-carbonyl)benzamido)hexan-2-yl)carbamoyl)-L-glutamate 23. Compound 22 (676 mg, 1.835 mmol) was warmed to 40° C. in DMF (40 mL) to form a solution. Di-tert-butyl (((S)-6-amino-1-(tert-butoxy)-1-oxohexan-2-yl)carbamoyl)-L-glutamate 19 (179 mg, 367 μmol) was dissolved in DMF (3 mL) and added to the solution of compound 23. The solution was allowed to cool to ambient temperature and after 2 hr solvents were removed in vacuo. The residue was suspended in dichloromethane (25 mL) and filtered using a medium fritted funnel. The solids were washed with dichloromethane (3×5 mL). Solvent was removed from the filtrate to a volume of ca. 20 mL and the suspension was filtered and washed with dichloromethane (2×2 mL). Solvents were removed and the residue was dried in vacuo. The residue was purified by silica gel chromatography using 2-3.5% isopropyl alcohol in dichloromethane. Product containing fractions were combined and solvents were removed under reduced pressure to provide compound 23 (107 mg, 39.6%).

16-(2-(4-(((S)-6-(tert-Butoxy)-5-(3-((S)-1,5-di-tert-butoxy-1,5-dioxopentan-2-yl)ureido)-6-oxohexyl)carbamoyl)benzamido)ethyl)-2,10,12,20-tetraoxo-1⁶,11⁶-bis((2-(trimethylsilyl)ethyl)thio)-6-oxa-3,9,13,16,19-pentaaza-1,11(2,5)-dipyridin-1-iumacycloicosaphane 1¹,11¹-dioxide 24. Compound 17 (12.86 mg, 14.6 umol) was dissolved in dry DCM (600 μL) and triethylamine (12.2 μL). Compound 23 (21 mg, 28.5 μmol) was dissolved in DCM (400 μL). The solution of compound 17 was added to the solution of compound 23 and allowed to stir for 2.5 hr. Solvents were removed under reduced pressure and the residue was dried overnight in vacuo. The residue was purified by silica gel chromatography using 2-3.5% isopropyl alcohol in dichloromethane to remove unreacted compound 23. The solvent was changed to 5% isopropyl alcohol in dichloromethane and then 5% methanol in dichloromethane to elute product. Product containing fractions were combined and solvents were removed under reduced pressure to provide compound 24 (20 mg, 96%). FTMS+pESI: Calc. for C₆₆H₁₀₄N₁₁O₁₆S₂Si₂ (M+H)⁺, 1426.6648; found, 1426.6648.

16-(2-(4-(((S)-5-carboxy-5-(3-((S)-1,3-Dicarboxypropyl)ureido)pentyl)carbamoyl)benzamido)ethyl)-1⁶,11⁶-dimercapto-2,10,12,20-tetraoxo-6-oxa-3,9,13,16,19-pentaaza-1,11(2,5)-dipyridin-1-iumacycloicosaphane 1¹,11¹-dioxide Compound 24 (17 mg, 12 μmol) was dissolved in a mixture of 5% triisopropylsilane, 50% trifluoroacetic acid in dichloromethane (1.0 mL). The resulting solution was stirred under a nitrogen atmosphere for 20 hours. Solvents were removed under reduced pressure and the product was dried in vacuo overnight. The residue was dissolved in methanol (400 μL) and transferred to an o-ring type 2 mL microcentrifuge tube. Diethyl ether (1.5 mL) was added to form a precipitate. The tube was centrifuged at 13,000 rpm for 3 minutes, the supernatant removed, and the pellet was washed with diethyl ether (1.5 mL). The tube was centrifuged at 13,000 rpm for 3 minutes, the supernatant removed, and the pellet was air dried 10 minutes then dried in vacuo to provide compound 25 as the trifluoroacetate salt (8.8 mg, 63%). FTMS-pESI: Calc. for C₄₄H₅₄N₁₁O₁₆S₂ (M−H)⁻, 1056.3197; found, 1056.3188.

Zinc complex of 16-(2-(4-(((S)-5-carboxy-5-(3-((S)-1,3-dicarboxypropyl)ureido)pentyl)carbamoyl)benzamido)ethyl)-1⁶,11⁶ -dimercapto-2,10,12,20-tetraoxo-6- oxa-3,9,13,16,19-pentaaza-1,11(2,5)-dipyridin-1-iumacycloicosaphane 1¹,11¹-dioxide 26. Compound 24 (18 mg, 13 μmol) was dissolved in a mixture of 5% triisopropylsilane, 50% trifluoroacetic acid in dichloromethane (1.0 mL). The resulting solution was stirred under a nitrogen atmosphere for 21 hours. Solvents were removed under reduced pressure and the product was dried in vacuo overnight. The residue was dissolved in methanol (500 μL) and transferred to an o-ring type 2 mL microcentrifuge tube. A solution of zinc(II) acetate in dimethylformamide (40 mM, 357 μL) was added and the resulting solution was divided into 2 o-ring type 2 mL microcentrifuge tubes. Diethyl ether (1.7 mL per tube) was added to form a precipitate. The tubes were centrifuged at 13,000 rpm for 3 minutes, the supernatant removed, and the pellets were washed with diethyl ether (1.5 mL). The tubes were centrifuged at 13,000 rpm for 3 minutes, the supernatant removed, and the pellets were air dried 10 minutes then dried in vacuo to provide the zinc complex 26 (11.9 mg, 76%). FTMS+pESI: Calc. for C₄₄H₅₄N₁₁O₁₆S₂Zn (M+H)⁺, 1120.2488; found, 1120.2500.

Copper complex of 16-(2-(4-(((S)-5-carboxy-5-(3-((S)-1,3-dicarboxypropyl)ureido)pentyl)carbamoyl)benzamido)ethyl)-1⁶,11⁶-dimercapto-2,10,12,20-tetraoxo-6- oxa-3,9,13,16,19-pentaaza-1,11(2,5)-dipyridin-1-iumacycloicosaphane 1¹,11¹-dioxide 27. Compound 26 (5.14 mg, 4.16 μmol) in a centrifuge tube was dissolved using a solution of copper acetate in dimethylformamide (40 mM, 104 μL, 4.16 μmol) and shaken to form a suspension. The tube was centrifuged at 13,000 rpm for 1 minute, the supernatant removed, and the pellet was washed with methanol (0.5 mL). The resulting pellet was dissolved in dimethylsulfoxide (40 uL) and triethylammonium bicarbonate buffer (0.1M, pH 7.6, 2 mL) and purified by HPLC using a gradient (15 — 35%) of acetonitrile in triethylammonium bicarbonate buffer. Product fractions were collected, combined, and lyophilized to provide copper complex 27. FTMS-pESI: Calc. for C₄₄H₅₂N₁₁O₁₆S₂Cu (M−H)⁻, 1117.2336; found, 1117.2336.

### Example 7

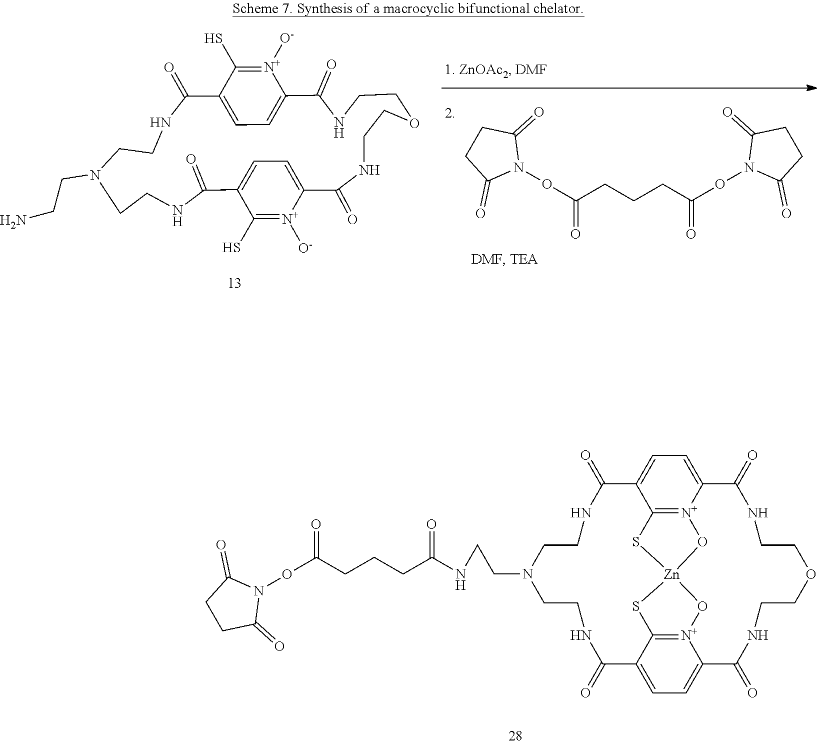

Zinc complex of 16-(2-(5-((2,5-dioxopyrrolidin-1-yl)oxy)-5-oxopentanamido)ethyl)-1⁶,1¹-6 -dimercapto-2,10,12,20-tetraoxo-6-oxa-3,9,13,16,19-pentaaza- 1,11(2,5)-dipyridin-1-iumacycloicosaphane 1¹,1¹¹-dioxide 28. Compound 13 (11.4 mg, 15.8 μmol) was dissolved in dimethylformamide (250 μL). A solution of zinc acetate in dimethylformamide (40 mM, 17.4 μmol, 434 μL) was added, and a solution of disuccinimidyl glutarate (51.5 mg, 158 μmol) in dimethylformamide (250 μL) and triethylamine (158 μmol, 22 μL) were added sequentially to the resulting suspension. The suspension was shaken for 1.5 hr, whereupon the reaction mixture was partitioned into four 2-mL microcentrifuge tubes. Diethyl ether (1.5 mL) was added to each tube, the contents mixed, and centrifuged at 12,000 rpm at once for 3 minutes. The supernatents were removed, the residues were washed with diethyl ether, and the pellets were air dried briefly then dried in vacuo. Each pellet was dissolved in dimethylformamide (250 μL), diethyl ether (1 mL) was added, the resulting suspensions were centrifuged, and the pellets were washed with ethyl ether as above. Each pellet was dissolved again in dimethylformamide (250 μL), diethyl ether (1 mL) was added, the resulting suspensions were centrifuged, and the pellets were washed with ethyl ether as above. The pellets were dried in vacuo to provide compound 28 (13.51 mg, 97.0%). FTMS+pESI: Calc. for C₃₃H₄₀N₉O₁₂S₂Zn (M+H)⁺, 882.1524; found, 882.1525.

### Example 8

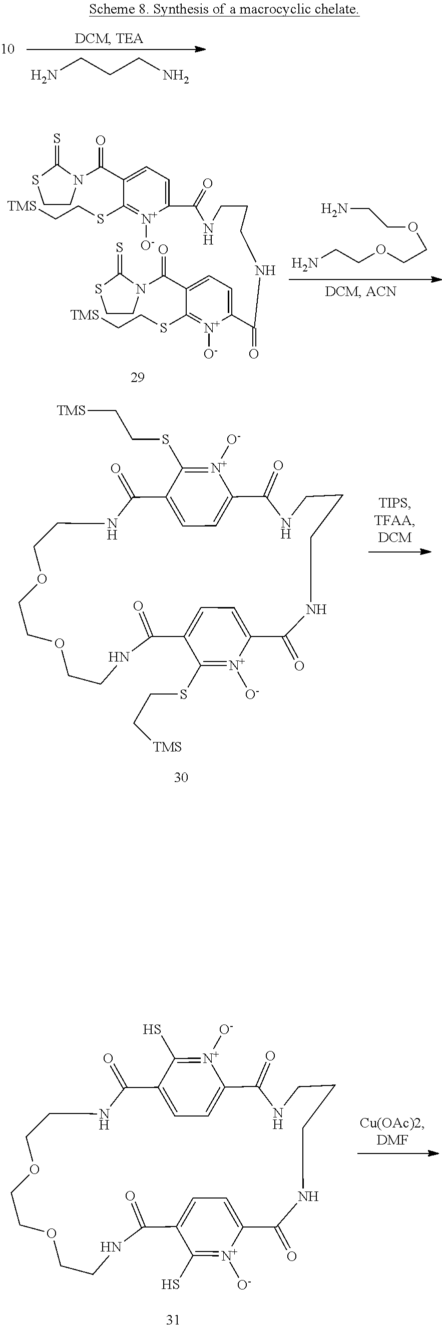

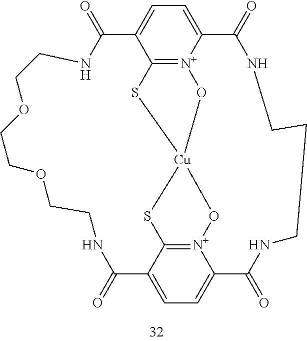

6,6′-((Propane-1,3-diylbis(azanediyl))bis(carbonyl))bis(3-(2-thioxothiazolidine-3-carbonyl)-2-((2-(trimethylsilyl)ethyl)thio)pyridine 1-oxide) 29. Compound 10 (600 mg, 1.159 mmol) was dissolved in anhydrous dichloromethane (25 mL) and cooled on an ice bath. 1,3-diaminopropane (Tokyo Chemicals, 38.7 mg, 521 μmol) and triethylamine (218 μL, 1.56 mmol) were dissolved in dichloromethane (10 mL) and the solution was transferred to a syringe. The solution was added to the cooled solution of compound 10 in portions over 30 minutes. The solution was allowed to slowly warm to ambient temperature overnight. After 18 hours, solvents were reduced to ca. 2 mL. The solution was purified using silica gel chromatography, using 1-3.5% isopropyl alcohol in dichloromethane to elute compound 29 that formed an oily yellow solid upon removal of solvent and that was dried in vacuo (508 mg, 101%). FTMS+pESI: Calc. for C₃₃H₄₇N₆O₆S₆Si₂ (M+H)⁺, 871.1414; found, 871.1422.

2,8,10,21-tetraoxo-1⁶,9⁶-bis((2-(trimethylsilyl)ethyl)thio)-14,17-dioxa-3,7,11,20-tetraaza-1,9(2,5)-dipyridin-1-iumacyclohenicosaphane 1¹,9¹-dioxide 30. Compound 29 (432 mg, 496 μmol) was dissolved in anhydrous dichloromethane and anhydrous acetonitrile (1:1 mixture, mL) and transferred to a 50 mL Hamilton syringe. 1,2-Bis(2-aminoethoxy)ethane (Tokyo Chemical, 73 mg, 496 μmol) was dissolved in anhydrous acetonitrile (30 mL) and transferred to another syringe. The two reactants were added to a stirring pot of dichloromethane (500 mL) at a rate of 0.50 mL/hr over 30 hours using two syringe pumps. Triethylamine (207 μL, 150 mg, 1.49 mmol) was added to the reaction mixture at this time. Unreacted compound 29 present in the syringe (ca. 15 mL) was recovered and the reaction mixture was allowed to stir for an additional four days. Solvents were removed and the residue was dried overnight. The residue was purified using silica gel chromatography, using 0.1% triethylamine, 2-5% methanol in dichloromethane to elute compound 30 that formed an off white solid upon removal of solvent and that was dried in vacuo (97 mg, 50%). FTMS+pESI: Calc. for C₃₃H₅₃N₆O₈S₂Si₂ (M+H)⁺, 781.2899; found, 781.2910.

1⁶,9⁶-Dimercapto-2,8,10,21-tetraoxo-14,17-dioxa-3,7,11,20-tetraaza-1,9(2,5)-dipyridin-1-iumacyclohenicosaphane 1¹,9¹-dioxide 31. Compound 30 (97 mg, 124 μmol) was dissolved in a mixture of 5% triisopropylsilane, 50% trifluoroacetic acid in dichloromethane (2.0 mL). The resulting solution was stirred under a nitrogen atmosphere for 42 hours. Solvents were removed under reduced pressure and the product was dried in vacuo overnight. The residue was dissolved in methanol (1.4 mL) and dichloromethane (2.4 mL) and transferred to a 50 mL centrifuge tube using additional dichloromethane (0.6 mL) to complete the transfer. Diethyl ether (40 mL) was added to form a precipitate. The tube was centrifuged at 1,500 rpm for 5 minutes, the supernatant removed, and the pellet was suspended in diethyl ether (8 mL). The suspension was transferred to four 2 mL microcentrifuge tubes. The tubes were centrifuged at 13,000 rpm for 3 minutes, the supernatant removed, and the pellets were air dried 10 minutes then dried in vacuo to provide compound 31 (56.9 mg, 79.1%). FTMS+pESI: Calc. for C₂₃H₂₉N₆O₈S₂ (M+H)⁺, 581.1483; found, 581.1480.

1⁶,9⁶-Dimercapto-2,8,10,21-tetraoxo-14,17-dioxa-3,7,11,20-tetraaza-1,9(2,5)-dipyridin-1-iumacyclohenicosaphane 1¹,9¹-dioxide, copper complex 32. Compound 31 is dissolved in dimethylformamide and a solution of copper(II) acetate (40 mM in dimethylformamide) is added. The resulting precipitate of compound 32 that forms is dark green in color.

### Example 9

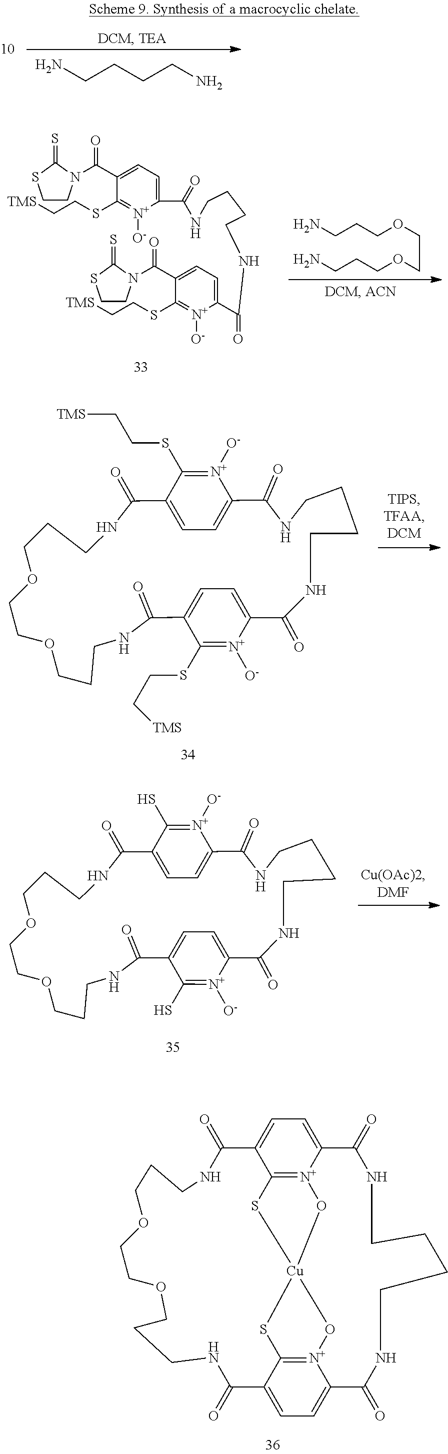

6,6′-((Butane-1,4-diylbis(azanediyl))bis(carbonyl))bis(3-(2-thioxothiazolidine-3-carbonyl)-2-((2-(trimethylsilyl)ethyl)thio)pyridine 1-oxide) 33. Compound 10 (1000 mg, 1.931 mmol) was dissolved in anhydrous dichloromethane (42 mL) and cooled on an ice bath. 1,4-diaminobutane (Tokyo Chemicals, 76.6 mg, 869 μmol) and triethylamine (363 μL, 2.60 mmol) were dissolved in dichloromethane (17 mL) and the solution was transferred to a syringe. The solution was added to the cooled solution of compound 10 in portions over 30 minutes. The solution was allowed to slowly warm to ambient temperature overnight. After 23 hours, solvents were removed under reduced pressure. The residue was purified using silica gel chromatography, using 1-3.5% isopropyl alcohol in dichloromethane to elute compound 33 to form an oily yellow solid upon removal of solvent and that was dried in vacuo (710 mg, 92.3%). FTMS+pESI: Calc. for C₃₄H₄₉N₆O₆S₆Si₂ (M+H)⁺, 885.1571; found, 885.1588.

2,9,11,24-Tetraoxo-1⁶,10⁶-bis((2-(trimethylsilyl)ethyl)thio)-16,19-dioxa-3,8,12,23-tetraaza-1,10(2,5)-dipyridin-1-iumacyclotetracosaphane 1¹,10¹-dioxide 34. Compound 33 (368 mg, 416 μmol) was dissolved in anhydrous dichloromethane (50 mL) and transferred to a 50 mL Hamilton syringe. Ethylene glycol bis(3-aminopropyl) ether (Tokyo Chemical, 73 mg, 416 μmol) was dissolved in anhydrous acetonitrile (50 mL) and transferred to another syringe. The two reactants were added to a stirring pot of dichloromethane (500 mL) at a rate of 0.50 mL/hr over 100 hours using two syringe pumps. The reaction mixture was allowed to stir for a total of five days after the start of addition. Solvents were removed and the residue was dried overnight. The residue was purified using silica gel chromatography, using 0.1% triethylamine, 2-5% methanol in dichloromethane to elute compound 34 to form an off white solid upon removal of solvent and that was dried in vacuo (227 mg, 66.3%). FTMS+pESI: Calc. for C₃₆H₅₉N₆O₈S₂Si₂ (M+H)⁺, 823.3369; found, 823.3362.

1⁶,10⁶-dimercapto-2,9,11,24-tetraoxo-16,19-dioxa-3,8,12,23-tetraaza-1,10(2,5)-dipyridin-1-iumacyclotetracosaphane 1¹,10¹-dioxide 35. Compound 34 (121 mg, 147 μmol) was dissolved in a mixture of 5% triisopropylsilane, 50% trifluoroacetic acid in dichloromethane (2.0 mL). The resulting solution was stirred under a nitrogen atmosphere for 48 hours. Solvents were removed under reduced pressure and the product was dried in vacuo overnight. The residue was suspended in methanol (4 mL) and dichloromethane (4 mL) and transferred to 8-2 mL centrifuge tubes. Diethyl ether (ca. 1.0 mL/tube) was added to form precipitate. The tubes were centrifuged at 13,000 rpm for 1 minute, the supernatant removed and the pellets were washed with diethyl ether (1.5 mL/tube). The tubes were centrifuged at 13,000 rpm for 1 minute, the supernatants removed, and the pellets were air dried 10 minutes then dried in vacuo to provide compound 35 (67.3 mg, 73.5%). FTMS+pESI: Calc. for C₂₆H₅₃N₆O₈S₂ (M+H)⁺, 623.1952; found, 623.1953.

1⁶,10⁶-dimercapto-2,9,11,24-tetraoxo-16,19-dioxa-3,8,12,23-tetraaza-1,10(2,5)-dipyridin-1-iumacyclotetracosaphane 1¹,10¹-dioxide, copper complex 36. Compound 35 (12.52 mg, 20.1 μmol) was dissolved in dimethylformamide (600 μL) and a solution of copper(II) acetate (40 mM in dimethylformamide, 553 μL) was added. The resulting suspension of compound 36 that formed was dark green/brown in color. The suspension was divided between two 2 mL microtubes, and water (1000 μL) was added to each tube. The tubes were centrifuged for one minute at 13,000 rpm, the supernatant decanted, and the pellets were washed with water (1.5 mL per tube). The tubes were centrifuged, the supernatant decanted, and the pellets were dried in vacuo to provide compound 36 as a green solid (12.3 mg, 89.6%). FTMS+pESI: Calc. for C₂₆H₃₃N₆O₈S₂Cu (M+H)⁺, 684.1092; found, 684.1088.

### Example 10

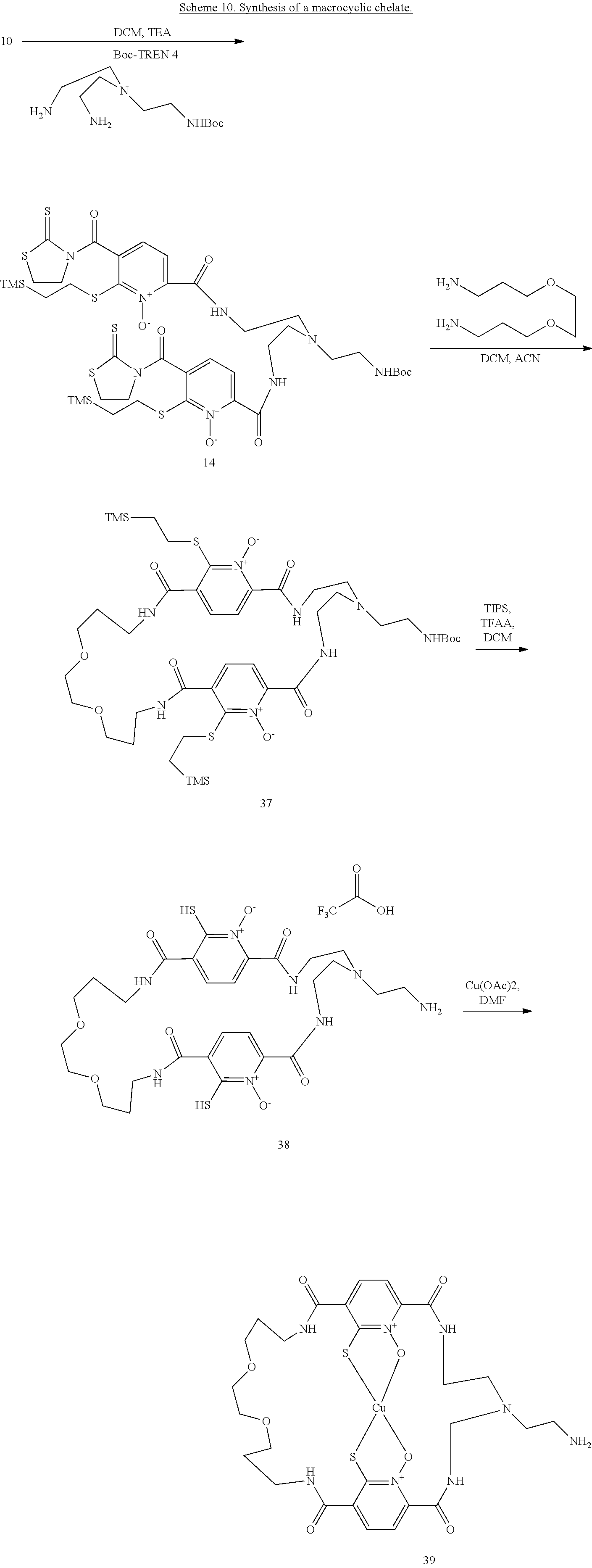

6-(2-((tert-Butoxycarbonyl)amino)ethyl)-2,10,12,25-tetraoxo-1⁶,11⁶-bis((2-(trimethylsilyl)ethyl)thio)-17,20-dioxa-3,6,9,13,24-pentaaza-1,11(2,5)-dipyridin-1-iumacyclopentacosaphane 1¹,11¹-dioxide 37. Compound 14 (301 mg, 288 μmol) was dissolved in anhydrous dichloromethane (50 mL) and transferred to a 50 mL Hamilton syringe. Ethylene glycol bis(3-aminopropyl) ether (Tokyo Chemical, 51 mg, 288 μmol) was dissolved in anhydrous acetonitrile (50 mL) and transferred to another syringe. The two reactants were added to a stirring pot of dichloromethane (500 mL) at a rate of 0.50 mL/hr over 100 hours using two syringe pumps. The reaction mixture was allowed to stir for a total of six days after the start of addition. Solvents were removed and the residue was dried overnight. The residue was purified using silica gel chromatography, using 3.5% isopropyl alcohol in dichloromethane initially, then changing to 0.1% triethylamine, 3.5-5% methanol in dichloromethane to elute compound 37 to form an off white solid upon removal of solvent and that was dried in vacuo (218 mg, 77.1%). FTMS+pESI: Calc. for C₄₃H₇₃N₈O₁₀S₂Si₂ (M+H)⁺, 981.4424; found, 981.4419.

6-(2-Aminoethyl)-1⁶,11⁶-dimercapto-2,10,12,25-tetraoxo-17,20-dioxa-3,6,9,13,24-pentaaza-1,11(2,5)-dipyridin-1-iumacyclopentacosaphane 1¹,11¹-dioxide, trifluoroacetate salt 38. Compound 37 (109 mg, 111 μmol) was dissolved in a mixture of 5% triisopropylsilane, 50% trifluoroacetic acid in dichloromethane (2.0 mL). The resulting solution was stirred under a nitrogen atmosphere for 7 days. Solvents were removed under reduced pressure and the product was dried in vacuo overnight. The residue was suspended in methanol (Trace Select, 1.7 mL) and transferred to four 2 mL centrifuge tubes. Diethyl ether (ca. 1.5 mL/tube) was added to form precipitate. The tubes were centrifuged at 13,000 rpm for 1 minute, the supernatant removed and the pellets were washed with diethyl ether (1.5 mL/tube). The tubes were centrifuged at 13,000 rpm for 1 minute, the supernatants removed, and the pellets were air dried 10 minutes then dried in vacuo to provide compound 38 as the trifluoroacetate salt (71.6 mg, 81.1%). FTMS+pESI: Calc. for C₂₈H₄₁N₈O₈S₂ (M+H)⁺, 681.2483; found, 681.2479.

Copper(II) complex of 6-(2-aminoethyl)-1⁶,11⁶-dimercapto-2,10,12,25-tetraoxo-17,20-dioxa-3,6,9,13,24-pentaaza-1,11(2,5)-dipyridin-1-iumacyclopentacosaphane 1¹,11¹-dioxide 39. Compound 38 (5.00 mg, 6.29 μmol) was dissolved in dimethylformamide (100 μL) and a solution of copper(II) acetate (40 mM in dimethylformamide, 188.7 μL, 7.55 μmol) was added. The resulting suspension that formed (dark green in color) formed a solution upon addition of trifluoroacetic acid (1.5 mL). The solution was filtered using a 0.45 μm nylon centrifugal filter and semiprep HPLC was used to purify the complex. Product containing fractions were combined and the resulting solution was lyophilized to provide the trifluoroacetate salt of compound 39 (4.4 mg, 82%). FTMS+pESI: Calc. for C₂₈H₃₉N₈O₈S₂Cu (M+H)⁺, 742.1623; found, 742.1624.

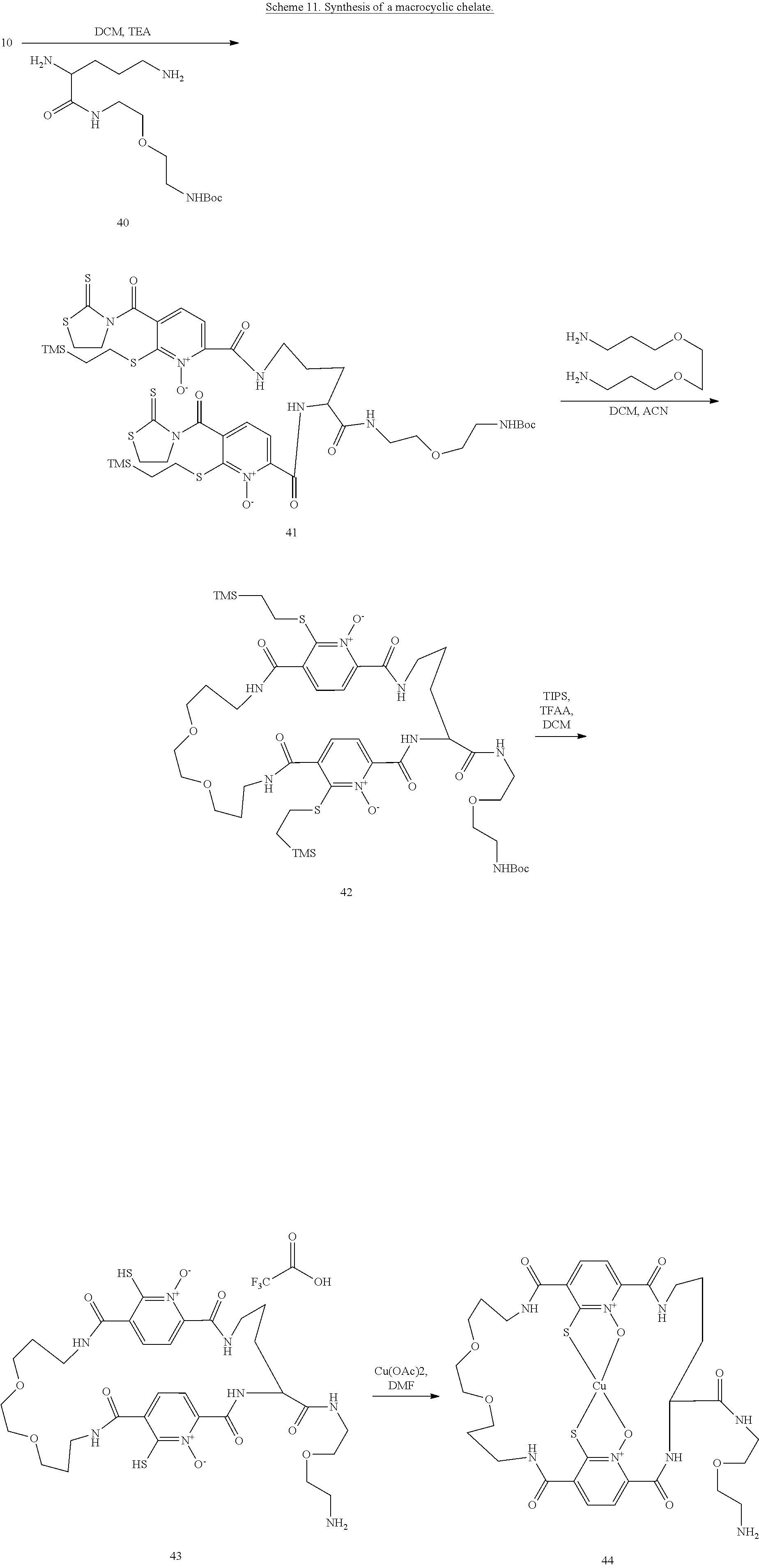

### Example 11

tert-Butyl (2-(2-(2,5-diaminopentanamido)ethoxy)ethyl)carbamate 40. A solution of (S)-2,5-bis(((benzyloxy)carbonyl)-amino)pentanoic acid (1.0 g, 2.5 mmol) in tetrahydrofuran (15 mL) was cooled to −25° C. and N-methyl morpholine (0.30 mL) was added. One minute later, isobutyl chloroformate (0.33 mL, 2.5 mmol) was added. About two minutes later, a solution of N-(tert-butoxycarbonyl)-2-(2-aminoethoxy)ethylamine (0.60 g, 2.5 mmol in 0.35 mL triethylamine and 5 mL dimethylformamide) was added slowly via a syringe to avoid a sudden temperature change. The reaction mixture was allowed to warm up to ambient temperature, whereupon solvents were removed under reduced pressure. The residue was treated with a mixture of water and dichloromethane (1:1, 25 mL), and the water phase was further extracted with dichloromethane (2×10 mL). The combined organic extracts were washed with brine (15 mL) then purified by silica gel column chromatography. Product containing fractions were combined, solvent removed under reduced pressure and the residue dried in vacuo to provide dibenzyl (2,2-dimethyl-4,12-dioxo-3,8-dioxa-5,11-diazahexadecane-13,16-diyl)dicarbamate as a white solid (1.05 g, 71.6%). FTMS+pESI: Calc. for C₃₀H₄₃N₄O₈ (M+H)⁺, 587.3075; found, 587.3076. Dibenzyl (2,2-dimethyl-4,12-dioxo-3,8-dioxa-5,11-diazahexadecane-13,16-diyl)dicarbamate (500 mg, 852 umol) was dissolved in methanol (15 mL). Palladium on carbon (10%, 50 mg) was added, and the atmosphere was exchanged six times for hydrogen using a balloon. After stirring overnight, the catalyst was removed by filtration using Celite 545. The Celite filter aid was washed with methanol (4 times with 20 mL) and the combined filtrate and methanol washes were dried under reduced pressure to provide compound 40 (267 mg, 98.4%). Compound 40 was used without further purification.

6-((17,17-Dimethyl-1-(1-oxido-5-(2-thioxothiazolidine-3-carbonyl)-6-((2-(trimethylsilyl)ethyl)thio)pyridin-2-yl)-1,7,15-trioxo-11,16-dioxa-2,8,14- triazaoctadecan-6-yl)carbamoyl)-3-(2-thioxothiazolidine-3-carbonyl)-2-((2-(trimethylsilyl)ethyl)thio)pyridine 1-oxide 41. Compound 10 (868 mg, 1.677 mmol) was dissolved in anhydrous dichloromethane (30 mL) and cooled on an ice bath. tert-Butyl (2-(2-(2,5-diaminopentanamido)ethoxy)ethyl)carbamate 40 (267 mg, 839 μmol) and triethylamine (351 μL, 2.52 mmol) were dissolved in dichloromethane (10 mL) and the solution was transferred to a syringe. The solution was added to the cooled solution of compound 10 in portions over 120 minutes. The solution was allowed to slowly warm to ambient temperature overnight. After 23.5 hours, solvents were removed under reduced pressure. The residue was purified twice using silica gel chromatography, using 1-5% isopropyl alcohol in dichloromethane to elute compound 41 that formed an oily yellow solid upon removal of solvent and that was dried in vacuo (572 mg, 61.1%). FTMS+pESI: Calc. for C₄₄H₆₇N₈O₁₀S₆Si₂ (M+H)⁺, 1115.2838; found, 1115.2868.

4-((2-(2-((tert-butoxycarbonyl)amino)ethoxy)ethyl)carbamoyl)-2,9,11,24-tetraoxo-1⁶,10⁶-bis((2-(trimethylsilyl)ethyl)thio)-16,19-dioxa-3,8,12,23-tetraaza-1,10(2,5)-dipyridin-1-iumacyclotetracosaphane 1¹,10¹-dioxide 42. Compound 41 (497 mg, 446 μmol) was dissolved in anhydrous dichloromethane (45 mL) and transferred to a 50 mL Hamilton syringe. Ethylene glycol bis(3-aminopropyl) ether (Tokyo Chemical, 79 mg, 446 μmol) was dissolved in anhydrous acetonitrile (45 mL) and transferred to another syringe. The two reactants were added to a stirring pot of dichloromethane (500 mL) at a rate of 0.50 mL/hr over 90 hours using two syringe pumps. The reaction mixture was allowed to stir for a total of seven days after the start of addition. Solvents were removed and the residue was dried overnight. The residue was purified using silica gel chromatography, using 3.5% isopropyl alcohol in dichloromethane initially, then changing to 0.1% triethylamine, 3.5-5% methanol in dichloromethane to elute compound 42 to form an off white solid upon removal of solvent and that was dried in vacuo (294 mg, 62.6%). FTMS+pESI: Calc. for C₄₆H₇₇N₈O₁₂S₂Si₂ (M+H)⁺, 1053.4635; found, 1053.4638.

4-((2-(2-Aminoethoxy)ethyl)carbamoyl)-1⁶,10⁶-dimercapto-2,9,11,24-tetraoxo-16,19-dioxa-3,8,12,23-tetraaza-1,10(2,5)-dipyridin-1-iumacyclotetracosaphane 1¹,10¹-dioxide, trifluoroacetate salt 43. Compound 42 (98 mg, 93 μmol) was dissolved in a mixture of 5% triisopropylsilane, 50% trifluoroacetic acid in dichloromethane (2.0 mL). The resulting solution was stirred under a nitrogen atmosphere for 4 days. Solvents were removed under reduced pressure and the product was dried in vacuo overnight. The residue was suspended in methanol (Trace Select, 2.0 mL) and transferred to four 2 mL centrifuge tubes. Diethyl ether (ca. 1.5 mL/tube) was added to form precipitate. The tubes were centrifuged at 13,000 rpm for 1 minute, the supernatant removed and the pellets were washed with diethyl ether (1.5 mL/tube). The tubes were centrifuged at 13,000 rpm for 1 minute, the supernatants removed, and the pellets were air dried 10 minutes then dried in vacuo to provide compound 43 as the trifluoroacetate salt (41.5 mg, 51.4%). FTMS+pESI: Calc. for C₃₁H₄₅(M+H)⁺, 753.2695; found, 753.2687.

Copper(II) complex of 4-((2-(2-aminoethoxy)ethyl)carbamoyl)-1⁶,10⁶-dimercapto-2,9,11,24-tetraoxo-16,19-dioxa-3,8,12,23-tetraaza-1,10(2,5)-dipyridin-1- iumacyclotetracosaphane 1¹,10¹-dioxide, trifluoroacetate salt 44. Compound 43 (3.11 mg, 3.59 μmol) was dissolved in dimethylformamide (74 μL) and a solution of copper(II) acetate (40 mM in dimethylformamide, 129 μL, 3.23 μmol) was added. The resulting suspension that formed (dark green in color) formed a solution upon addition of 0.1% trifluoroacetic acid (1.058 mL). The solution was filtered using a 0.45 μm nylon centrifugal filter and semiprep HPLC was used to purify the complex. Product containing fractions were combined and the resulting solution was lyophilized to provide the trifluoroacetate salt of compound 44 (3.12 mg, 93.7%). FTMS+pESI: Calc. for C₃₁H₄₃N₈O₁₀S₂Cu (M+H)⁺, 814.1834; found, 814.1823.

### Example 12

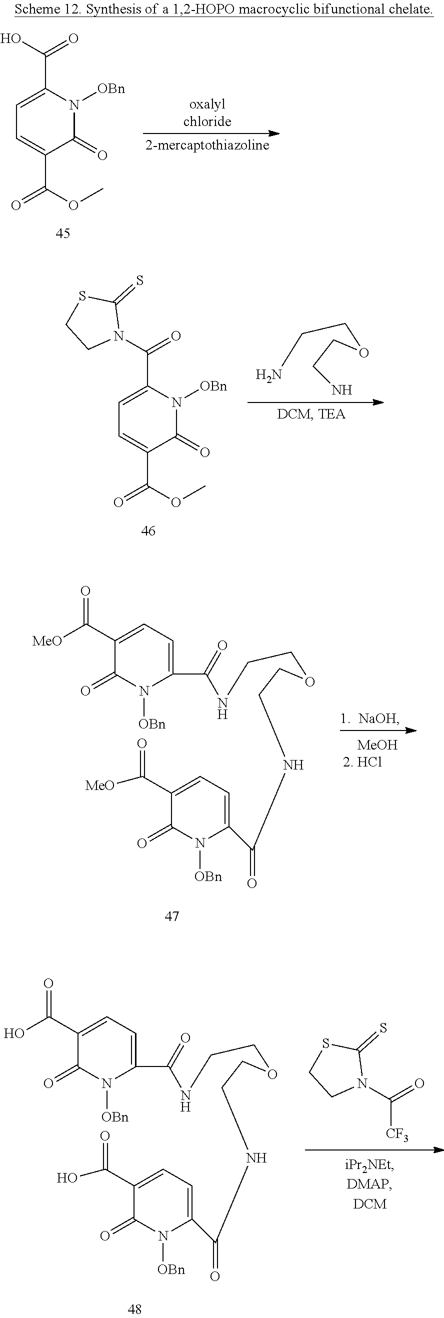

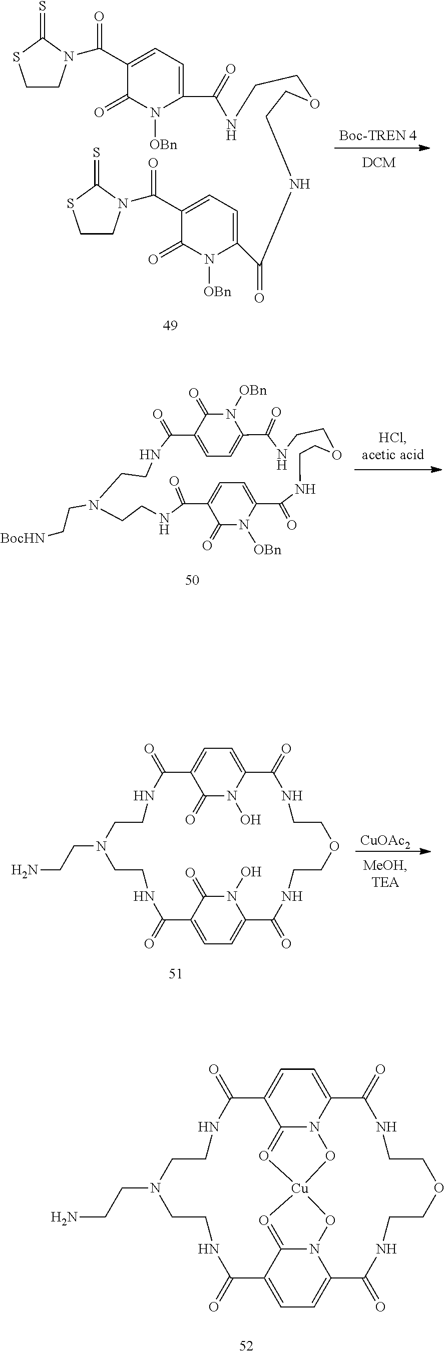

Methyl 1-(benzyloxy)-2-oxo-6-(2-thioxothiazolidine-3-carbonyl)-1,2-dihydropyridine-3-carboxylate 46. 1-(Benzyloxy)-5-(methoxycarbonyl)-6-oxo-1,6-dihydropyridine-2-carboxylic acid (9.203 g, 30.3 mmol) was suspended in dry dichloromethane (90 mL) and oxalyl chloride (8.47 g, 66.7 mmol) was added under an inert atmosphere. Dimethylformamide (25 μL) was added, causing the liberation of carbon dioxide gas. After 20 minutes, additional dimethylformamide (25 μL) was added. After a total time of 75 minutes, solvents were removed from the resulting solution under reduced pressure and the yellow residue was dried overnight in vacuo. Sodium bicarbonate (4.0 g) and potassium carbonate (8.0 g) were dissolved in water (100 mL) in a 500 mL round bottom flask. 2-Mercaptothiazoline (Acros Chemical, 5.43 g, 45.5 mmol) and dichloromethane (50 mL) were added to the flask and the mixture was stirred until the 2-mercaptothiazoline was mostly in solution. The mixture was cooled on an ice bath. The residue from above was dissolved in dry dichloromethane (90 mL) and transferred to a 125 mL addition funnel. This solution was added dropwise to the cooled mixture over the course of 75 minutes and allowed to stir overnight. The reaction mixture was transferred to a separatory funnel, the layers separated, and the dichloromethane layer was reduced in volume to ca. 75 mL. The solution was applied to a silica gel column and washed with dichloromethane until excess 2-mercaptothiazoline was removed. The product was eluted using 1% isopropyl alcohol in dichloromethane. Solvent was removed under reduced pressure to a volume of ca. 100 mL and tert-butyl methyl ether (ca. 300 mL) was added to form a yellow precipitate. The precipitate was filtered the following day and dried under reduced pressure to provide compound 46 (10.722 g, 87.5%) as a fluffy yellow solid. FTMS+pESI: Calc. for C₁₈H₁₇N₂O₅S₂ (M+H)⁺, 405.0573; found, 405.0572.

Dimethyl 6,6′-(((oxybis(ethane-2,1-diyl))bis(azanediyl))bis(carbonyl))bis(1-(benzyloxy)-2-oxo-1,2-dihydropyridine-3-carboxylate) 47. Compound 46 (1.80 g, 4.45 mmol) was dissolved in dry dichloromethane (75 mL). The solution was cooled on an ice bath. Oxybis(ethylamine), 2,2′-dihydrochloride (Tokyo Chemicals, 394 mg, 2.23 mmol) and triethylamine (675 mg, 6.67 mmol) were added sequentially, and the resulting solution was allowed to stir overnight. Solvents were removed under reduced pressure, and the residue was purified by silica gel chromatography using 1-2% isopropyl alcohol in dichloromethane to remove excess starting material, followed by 2-5% methanol in dichloromethane, 0.1% triethylamine, to elute product. Solvent was removed under reduced pressure and the residue was dried in vacuo overnight to provide compound 47 (1.484 g, 98.9%). FTMS+pESI: Calc. for C₃₄H₃₅N₄O₁₁ (M+H)⁺, 675.2297; found, 675.2299.

6,6′4(Oxybis(ethane-2,1-diyl))bis(azanediyl))bis(carbonyl))bis(1-(benzyloxy)-2-oxo-1,2-dihydropyridine-3-carboxylic acid) 48. Compound 47 (1.411 g, 2.09 mmol) was dissolved in tetrahydrofuran (10 mL). Water (2.5 mL) was added, and then 1M sodium hydroxide (4.60 mL, 4.6 mmol) was added. After ca. 3 hours, volatile solvent was removed from the resulting solution under reduced pressure. Water (10 mL) was added and 6N hydrochloric acid solution was added dropwise until the resulting suspension was acidic to pH paper. The precipitate was filtered, washed with water (5 mL), and dried in vacuo to provide compound 48 (1.260 g, 93.2%). FTMS-pESI: Calc. for C₃₂H₂₉N₄O₁₁ (M−H)⁻, 645.1838; found, 645.1843.

N,N′-(Oxybis(ethane-2,1-diyl))bis(1-(benzyloxy)-6-oxo-5-(2-thioxothiazolidine-3-carbonyl)-1,6-dihydropyridine-2-carboxamide) 49. Compound 48 (1.204 g, 1.86 mmol) and 4-dimethylaminopyridine (91 mg, 745 μmol) were dried in vacuo for 2 hours in a 100 mL round bottom flask. Dry dichloromethane (30 mL) was added to form a suspension, then N,N-diisopropylethylamine (1.45 g, 11.2 mmol) was added to form a solution. 2,2,2-Trifluoro-1-(2-thioxothiazolidin-3-yl)ethan-1-one (902 mg, ca. 567 μL, 4.19 mmol) was added using a pipette. The solution, which gradually turns from orange to yellow color, was allowed to stir for 4.5 hr. Solvents were removed and the residue was dried overnight. The residue was purified using silica gel chromatography, using 2-5% isopropyl alcohol in dichloromethane to elute product 49 that formed a yellow solid upon removal of solvent and that was dried in vacuo (1.400 mg, 88.6%). FTMS+pESI: Calc. for C₃₈H₃₇N₆O₉S₄ (M+H)⁺, 849.1499; found, 849.1508.

tert-Butyl (2-((1Z,11Z,14Z,24Z)-12,26-bis(benzyloxy)-2,10,13,15,23,25-hexaoxo-6-oxa-3,9,12,16,19,22,26-heptaazatricyclo[22.2.2.2^{11,14}]triaconta-1(27),11(30),14(29),24(28)-tetraen-19-yl)ethyl)carbamate 50. Compound 49 (657 mg, 774 μmol) was dissolved in anhydrous dichloromethane and anhydrous acetonitrile (1:1 mixture, 27 mL) and transferred to a 50 mL Hamilton syringe. tert-Butyl (2-(bis(2-aminoethyl)amino)ethyl)carbamate 4 (188 mg, 762 μmol) was dissolved in anhydrous acetonitrile (27 mL) and transferred to another syringe. The two reactants were added to a stirring pot of dichloromethane (500 mL) at a rate of 1.00 mL/hr over 27 hours using two syringe pumps. After stirring for an additional 20 hours, solvents were removed under reduced pressure and the residue was dried overnight. The residue was purified using silica gel chromatography, using 0.1% triethylamine, 2-5% methanol in dichloromethane to elute compound 50 that formed an off white solid upon removal of solvent and that was dried in vacuo (372 mg, 56.1%). FTMS+pESI: Calc. for C₄₃H₅₃N₈O₁₁ (M+H)⁺, 857.3828; found, 857.3818.

(1Z,11Z,14Z,24Z)-19-(2-Aminoethyl)-12,26-dihydroxy-6-oxa-3,9,12,16,19,22,26-heptaazatricyclo[22.2.2.211,14]triaconta-1(27),11(30),14(29),24(28)-tetraene-2,10,13,15,23,25-hexaone 51. Compound 50 (319 mg, 372 umol) was dissolved in acetic acid (10 mL). Hydrochloric acid (12N, 10 mL) was added, the flask was sealed and allowed to stand for 46 days. Hydrochloric acid was removed by a stream of air for 45 minutes, then solvent was removed under reduced pressure and dried in vacuo. The residue was dissolved in methanol (5 mL) and diethyl ether (15 mL) was added to form a precipitate. The precipitate was filtered, washed with diethyl ether (5 mL), and dried in vacuo to provide crude product (233 mg, quantitative). FTMS+pESI: Calc. for C₂₄H₃₃N₈O₉ (M+H)⁺, 577.2370; found, 577.2362.

Copper(II) complex of (1Z,11Z,14Z,24Z)-19-(2-aminoethyl)-12,26-dihydroxy-6-oxa-3,9,12,16,19,22,26-heptaazatricyclo[22.2.2.2^{11,14}]triaconta-1(27),11(30),14(29),24(28)-tetraene-2,10,13,15,23,25-hexaone 52. Compound 52 (10.0 mg, 16.3 μmol) was dissolved in 0.1% trifluoroacetic acid (1000 μL) and a solution of copper(II) acetate (40 mM in dimethylformamide, 408 μL, 16.3 μmol) was added. Additional 0.1% trifluoroacetic acid (2.6 mL) was added, the solution was filtered using a 0.45 μm nylon centrifugal filter, and semiprep HPLC was used to purify the complex. Product containing fractions were combined and the resulting solution was lyophilized to provide the trifluoroacetate salt of compound 52 (9.22 mg, FTMS+pESI: Calc. for C₂₄H₃₁N₈O₉Cu (M+H)⁺, 638.1505; found, 638.1504.

### Example 13

Stability testing of macrocyclic copper chelates. (FIG. 1).

Example 13. Copper complexes 27, 39, and 44 were dissolved at approximately 1 mM concentration in 100 mM Tris-HCl buffer, pH 7.5. A solution of 55 mM diethylenetriaminepentaacetic acid (DTPA), pH 7.5 was prepared. Copper complexes were analyzed by reversed phase HPLC using an Agilent 1200 Series instrument equipped with a Zorbax Eclipse XDB-C18 4.6×150 mm 5 μm column. The method used an isocratic gradient of 0.1M triethylammonium bicarbonate, pH 7.5 (Solvent A) and 10-80% acetonitrile (Solvent B) as mobile phases. A solution of copper complex and DTPA solution combined in a ratio of one to one was prepared and mixed at ambient temperature for 30 minutes prior to analysis. Admixture of DTPA and copper complexes led to variable levels of dissociation of the copper complex, as quantified by the elution of the metal free ligand at shorter retention time. Based upon the observed data, one can estimate that complex 44 is more stable in the presence of DTPA competitor than complex 39, and both are more stable than compound 27.

### Example 14

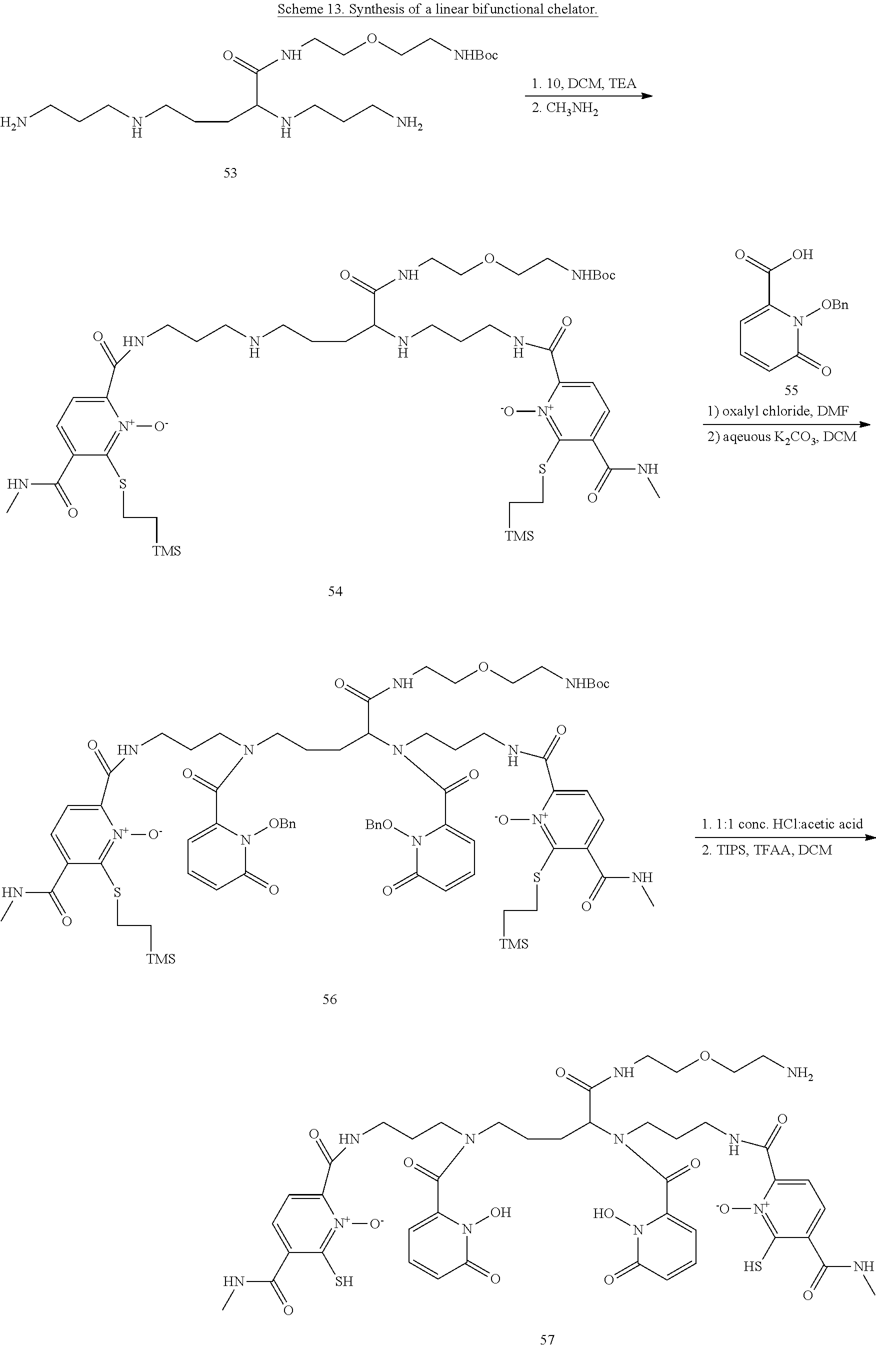

### Example 14. Synthesis of a Linear Bifunctional Chelator (Scheme 13)

Compound 10 is dissolved in dichloromethane and cooled on ice. Compound 53 (Butlin N G, Magda D, Xu J. Functionalized linear ligands and complexes thereof, U.S. Pat. No. 10,434,186, 2019) is dissolved in dichloromethane and triethylamine, and the solution is added gradually to the solution of compound 10. Once the reaction is judged to be complete using HPLC, a solution of 40% aqueous methylamine is added. The intermediate 54 is isolated by silica gel chromatography. Compound 55 is converted to its acid chloride form using thionyl chloride as described (Tatum D, Xu J, Magda D, Butlin N. Macrocyclic ligands with pendant chelating moieties and complexes thereof, WO/2019/173639, 2019: USA). The acid chloride is dissolved in dichloromethane and added dropwise to a stirred mixture of compound 54 in dichloromethane and aqueous potassium carbonate. The organic layer is isolated using a separatory funnel, and intermediate 56 is isolated by silica gel chromatography. Compound 56 is treated sequentially with hydrochloric acid in acetic acid and triispropylsilane, trifluoroacetic acid, dichloromethane mixture to remove protective groups. The product 57 is isolated by precipitation from methanol with diethyl ether.

### Example 15

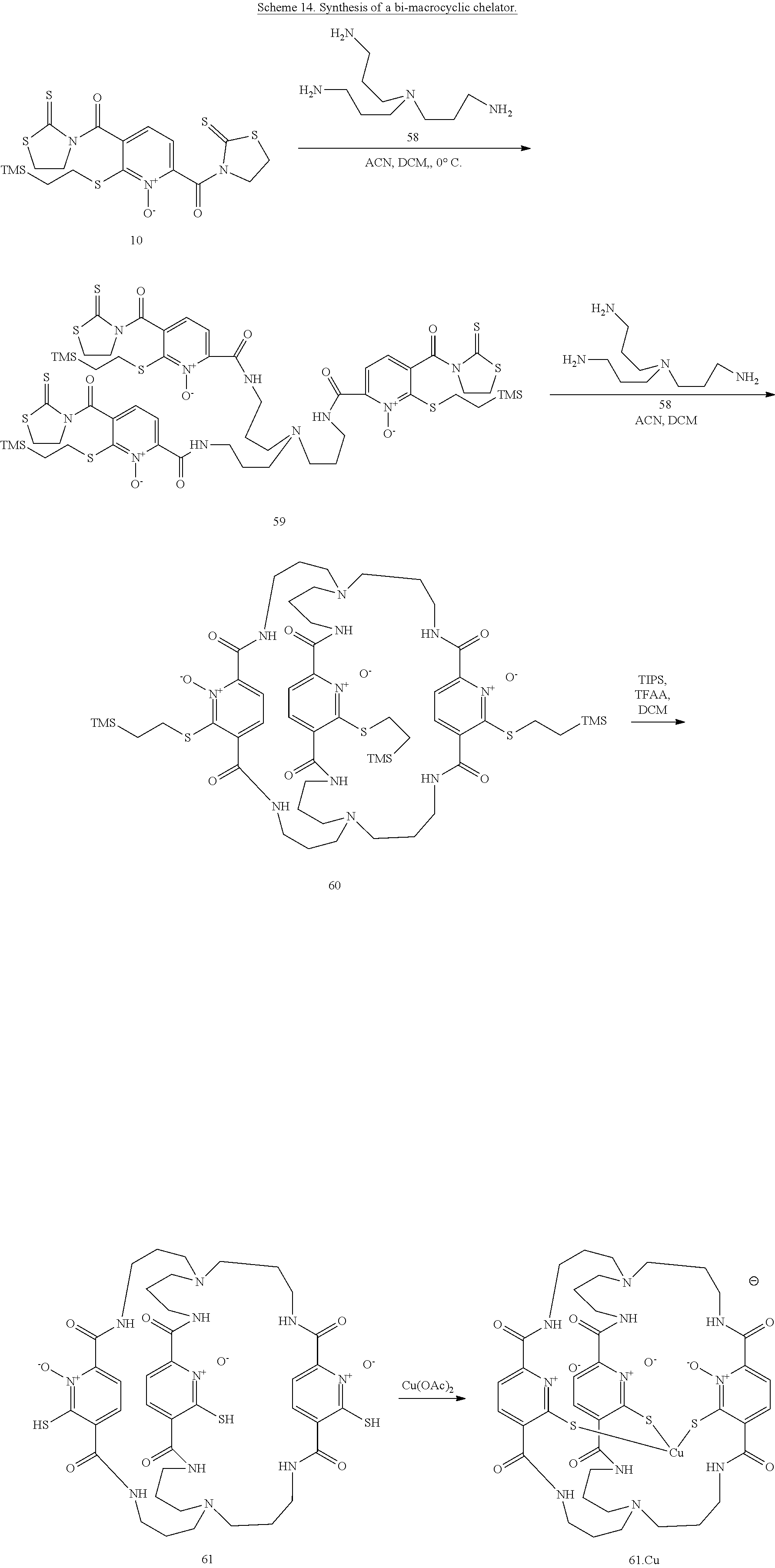

### Example 15. Synthesis of a Bi-Macrocyclic Chelator (Scheme 14)

Compound 10 is dissolved in dichloromethane and cooled on ice. Tris(3-aminopropyl)amine 58 (one third molar equivalent) is dissolved in acetonitrile and triethylamine, and the solution is added gradually to the solution of compound 10. Once the reaction is judged to be complete using HPLC, solvent is removed under reduced pressure and the intermediate 59 is purified by silica gel chromatography using isopropyl alcohol in dichloromethane. A solution of compound 59 in dichloromethane (50 mL) and an equimolar solution of compound 58 in acetonitrile (50 mL) are added to dichloromethane (1 L) dropwise over 100 hours using syringe pumps. Solvents are removed under reduced pressure, and the residue is purified by silica gel chromatography using methanol in dichloromethane with a small amount of triethylamine in the mobile phase to provide intermediate 60. Compound 60 is treated with a mixture of 5% triispropylsilane, 50% trifluoroacetic acid in dichloromethane to remove protective groups. The product 61 is isolated by precipitation from methanol with diethyl ether. Treatment of the methanolic solution of compound 61 with copper acetate converts it to the copper complex 61.Cu.

### Example 16

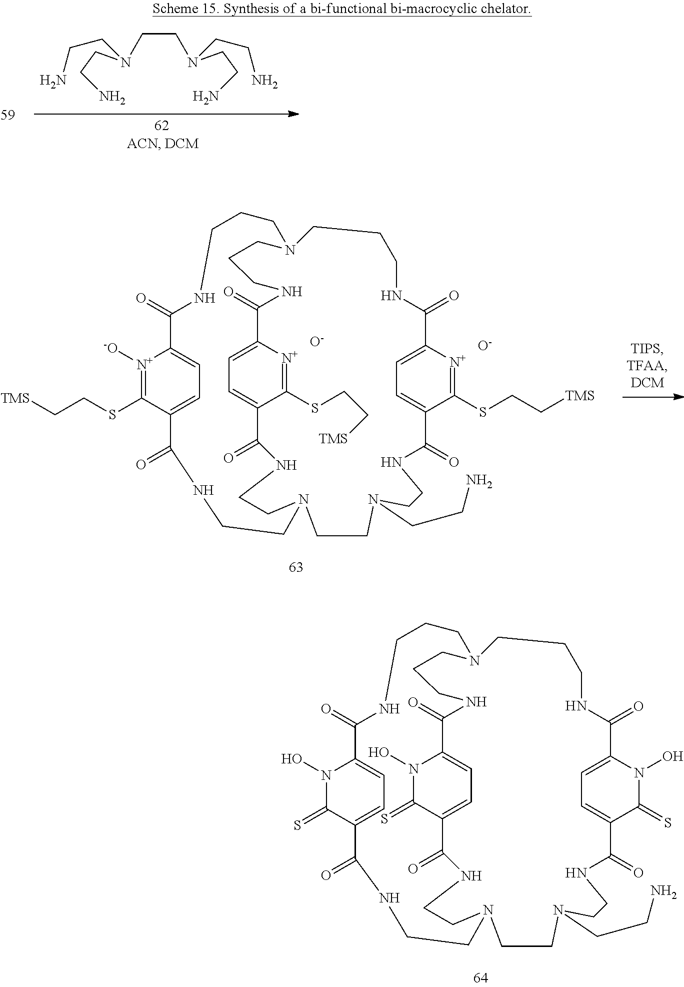

### Example 16. Synthesis of a Bi-Functional Bi-Macrocyclic Chelator (Scheme 15)

A solution of compound 59 in dichloromethane (50 mL) and an equimolar solution of compound 62 in acetonitrile (50 mL) are added to dichloromethane (1L) dropwise over 100 hours using syringe pumps. Solvents are removed under reduced pressure, and the residue is purified by silica gel chromatography using methanol in dichloromethane with a small amount of triethylamine in the mobile phase to provide intermediate 63. Compound 63 is treated with a mixture of 5% triispropylsilane, 50% trifluoroacetic acid in dichloromethane to remove protective groups. The product 64 is isolated by precipitation from methanol with diethyl ether.

### Example 17

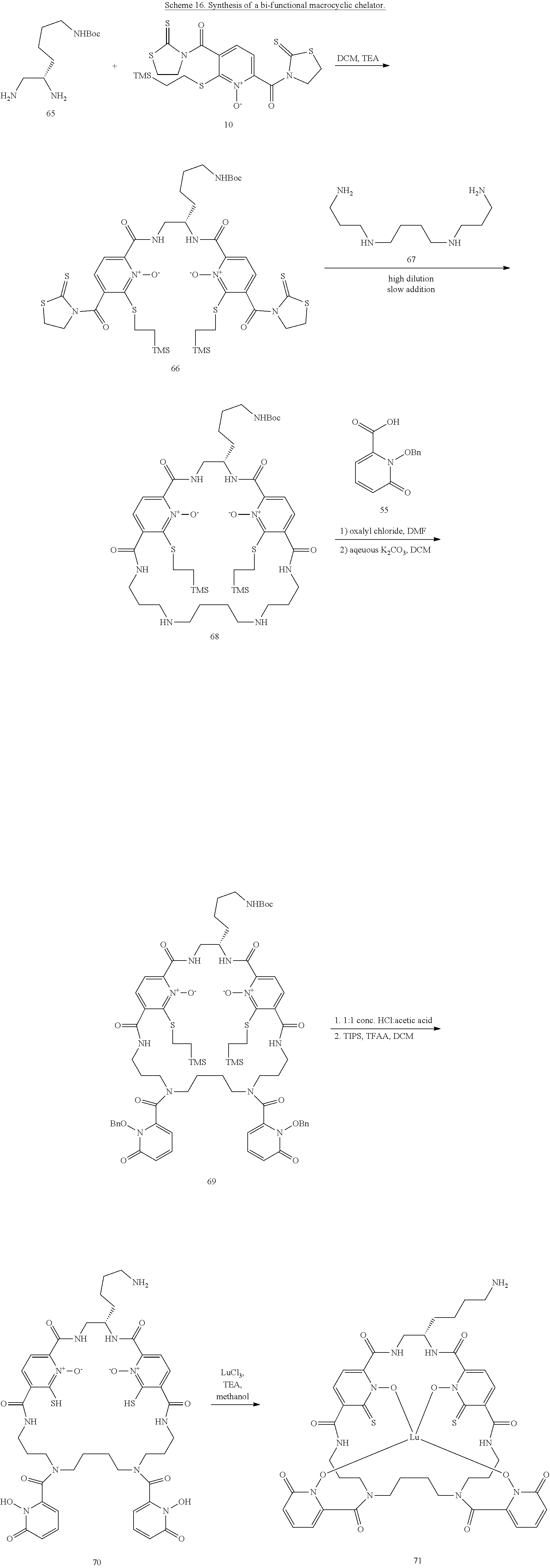

### Example 17. Synthesis of a Bi-Functional Macrocyclic Chelator (Scheme 16)

Compound 10 is dissolved in dichloromethane and cooled on ice. tert-Butyl (S)-(5,6-diaminohexyl)carbamate 65 (one half molar equivalent, prepared as described: Tatum D, Xu J, Magda D, Butlin N. Macrocyclic ligands with pendant chelating moieties and complexes thereof, WO/2019/173639, 2019: USA) is dissolved in acetonitrile and triethylamine, and the solution is added gradually to the solution of compound 10. Once the reaction is judged to be complete using HPLC, solvent is removed under reduced pressure and the intermediate 66 is purified by silica gel chromatography using isopropyl alcohol in dichloromethane. A solution of compound 66 in dichloromethane (50 mL) and an equimolar solution of spermine 67 in acetonitrile (50 mL) are added to dichloromethane (1 L) dropwise over 100 hours using syringe pumps. Solvents are removed under reduced pressure, and the residue containing crude compound 68 is dissolved in dichloromethane and aqueous potassium carbonate, and then treated dropwise with a dichloromethane solution containing eight molar equivalents of the acid chloride prepared from compound 55. The product is purified by silica gel chromatography using methanol in dichloromethane with a small amount of triethylamine in the mobile phase to provide intermediate 69. Compound 69 is treated sequentially with a mixture of hydrochloric acid in acetic acid and with a mixture of 5% triispropylsilane, 50% trifluoroacetic acid in dichloromethane to remove protective groups. The product 70 is isolated by precipitation from methanol with diethyl ether. Complexes with metal cations, such as the lutetium(III) complex 71, may be prepared by treatment of compound 70 with methanolic base and a methanolic solution of lutetium(III) chloride.

### Example 18

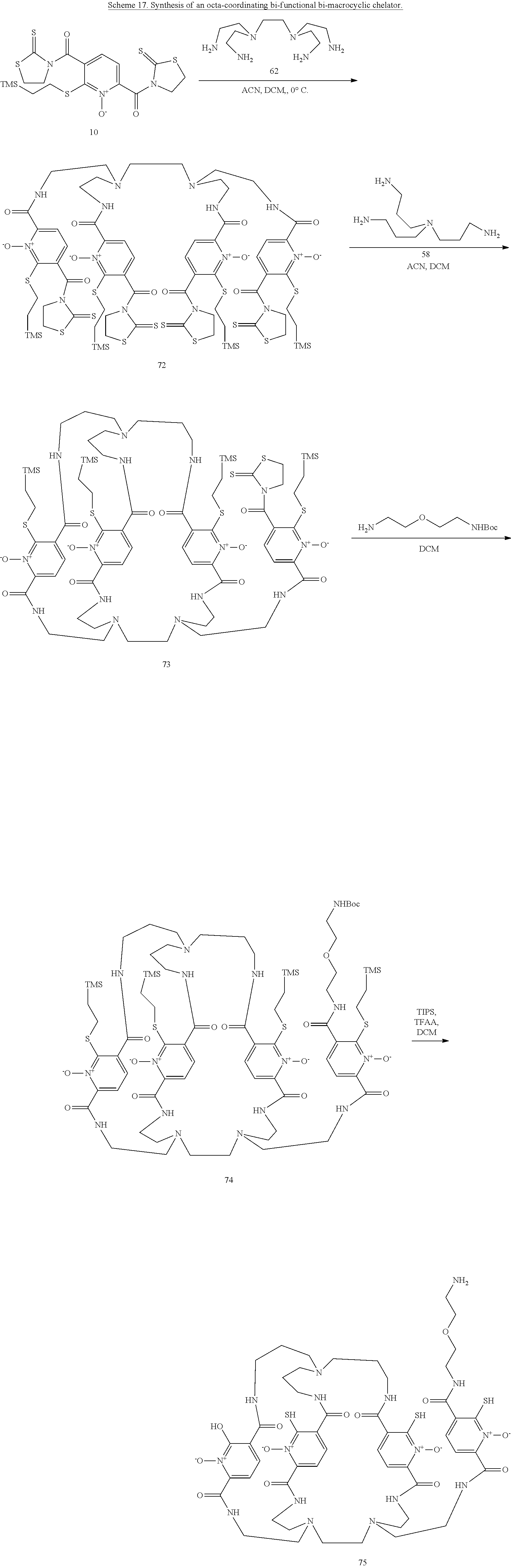

### Example 18. Synthesis of a Bi-Functional Bi-Macrocyclic Chelator (Scheme 17)

Compound 10 is dissolved in dichloromethane and cooled on ice. Amine 62 (one quarter molar equivalent) is dissolved in acetonitrile and triethylamine, and the solution is added gradually to the solution of compound 10. Once the reaction is judged to be complete using HPLC, solvent is removed under reduced pressure and the intermediate 72 is purified by silica gel chromatography using isopropyl alcohol in dichloromethane. A solution of compound 72 in dichloromethane (50 mL) and an equimolar solution of amine 58 in acetonitrile (50 mL) are added to dichloromethane (1L) dropwise over 100 hours using syringe pumps. Solvents are removed under reduced pressure, and the residue containing crude compound 73 is purified by silica gel chromatography using isopropyl alcohol in dichloromethane. Intermediate 73 is dissolved in dichloromethane, and a solution containing one molar equivalent of tert-butyl (2-(2-aminoethoxy)ethyl)carbamate in dichloromethane is added gradually. The product is purified by silica gel chromatography using methanol in dichloromethane with a small amount of triethylamine in the mobile phase to provide intermediate 74. Compound 74 is treated with a mixture of 5% triispropylsilane, 50% trifluoroacetic acid in dichloromethane to remove protective groups. The product 75 is isolated by precipitation from methanol with diethyl ether.

### Example 19

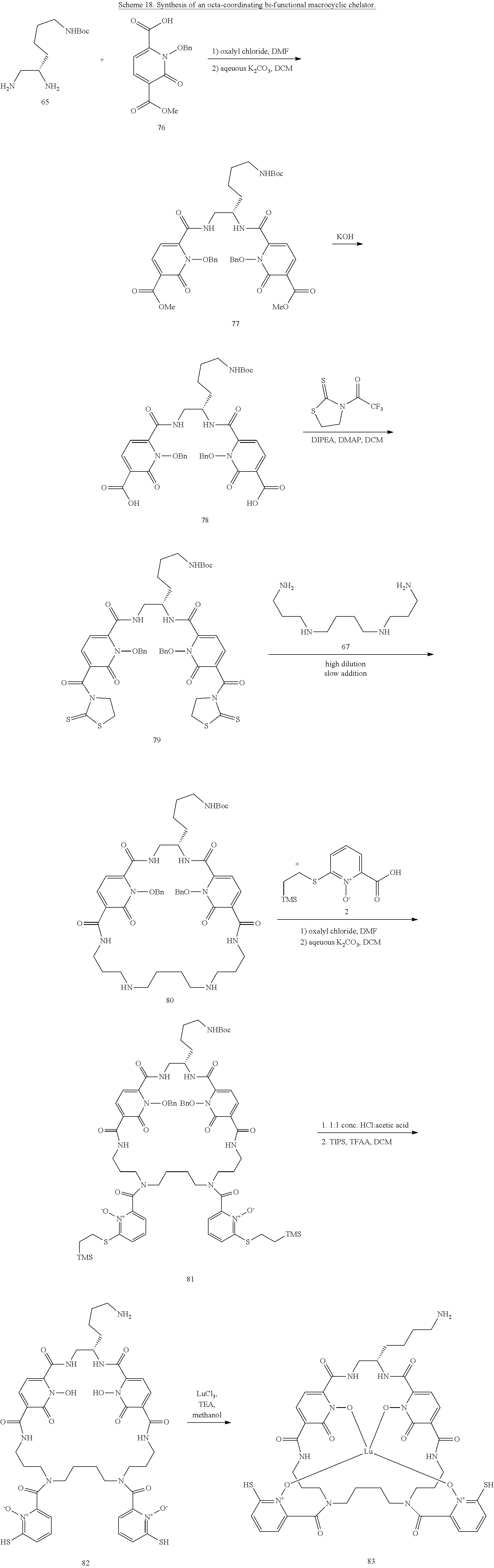

### Example 19. Synthesis of a Bi-Functional Macrocyclic Chelator (Scheme 18)

Crude compound 80 (Tatum D, Xu J, Magda D, Butlin N. Macrocyclic ligands with pendant chelating moieties and complexes thereof, WO/2019/173639, 2019: USA) is treated dropwise with a dichloromethane solution containing eight molar equivalents of the acid chloride prepared from compound 2. The product is purified by silica gel chromatography using methanol in dichloromethane with a small amount of triethylamine in the mobile phase to provide intermediate 81. Compound 81 is treated sequentially with a mixture of hydrochloric acid in acetic acid and with a mixture of 5% triispropylsilane, 50% trifluoroacetic acid in dichloromethane to remove protective groups. The product 82 is isolated by precipitation from methanol with diethyl ether. Complexes with metal cations, such as the lutetium(III) complex 83, may be prepared by treatment of compound 82 with methanolic base and a methanolic solution of lutetium(III) chloride.

### Example 20

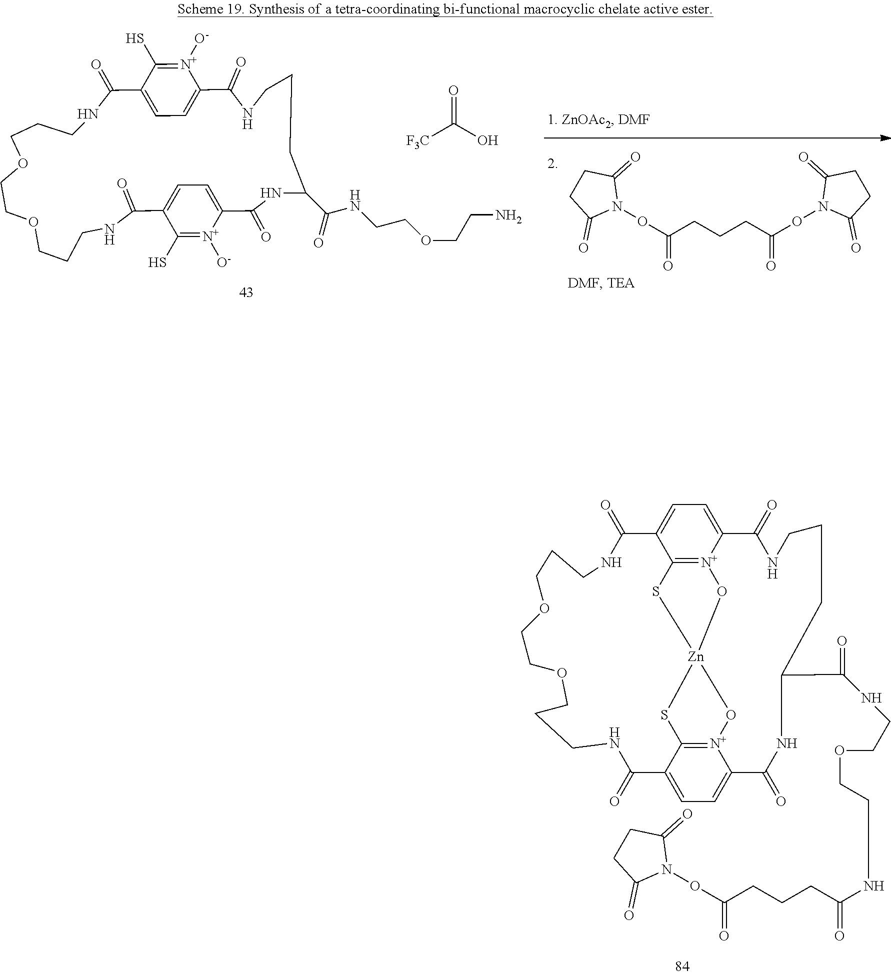

### Example 20. Synthesis of a Bi-Functional Macrocyclic Chelate Active Ester (Scheme 19)

Zinc complex of 4-((2-(2-(5-((2,5-dioxopyrrolidin-1-yl)oxy)-5-oxopentanamido)ethoxy)ethyl)carbamoyl)-1¹,10¹-dihydroxy-1⁶,10⁶-dimercapto-2,9,11,24-tetraoxo-16,19-dioxa-3,8,12,23-tetraaza-1,10(2,5)-dipyridin-1-iumacyclotetracosaphane-1¹,10¹-diium 84. Compound 43 (9.51 mg, 11.0 μmol) was dissolved in dimethylformamide (250 μL). A solution of zinc acetate in dimethylformamide (40 mM, 12.1 μmol, 302 μL) was added, and a solution of disuccinimidyl glutarate (35.8 mg, 110 μmol) in dimethylformamide (175 μL) and triethylamine (110 μmol, 15.3 μL) were added sequentially to the resulting suspension. The suspension was shaken for 2.2 hr, whereupon the reaction mixture was partitioned into four 2-mL microcentrifuge tubes. Diethyl ether (1.5 mL) was added to each tube, the contents mixed, and centrifuged at 11,000 rpm at once for 3 minutes. The supernatents were removed, the residues were washed with diethyl ether, and the pellets were air dried briefly then dried in vacuo. Each pellet was dissolved in dimethylformamide (125 μL), diethyl ether (1 mL) was added, the resulting suspensions were centrifuged, and the pellets were washed with ethyl ether as above. Each pellet was dissolved again in dimethylformamide (125 μL), diethyl ether (1 mL) was added, the resulting suspensions were centrifuged, and the pellets were washed with ethyl ether as above. The pellets were dried in vacuo to provide compound 84 (7.78 mg, 68.8%). FTMS+pESI: Calc. for C₄₀H₅₂N₉O15S₂Zn (M+H)⁺, 1026.2310; found, 1026.2307.

### Example 21

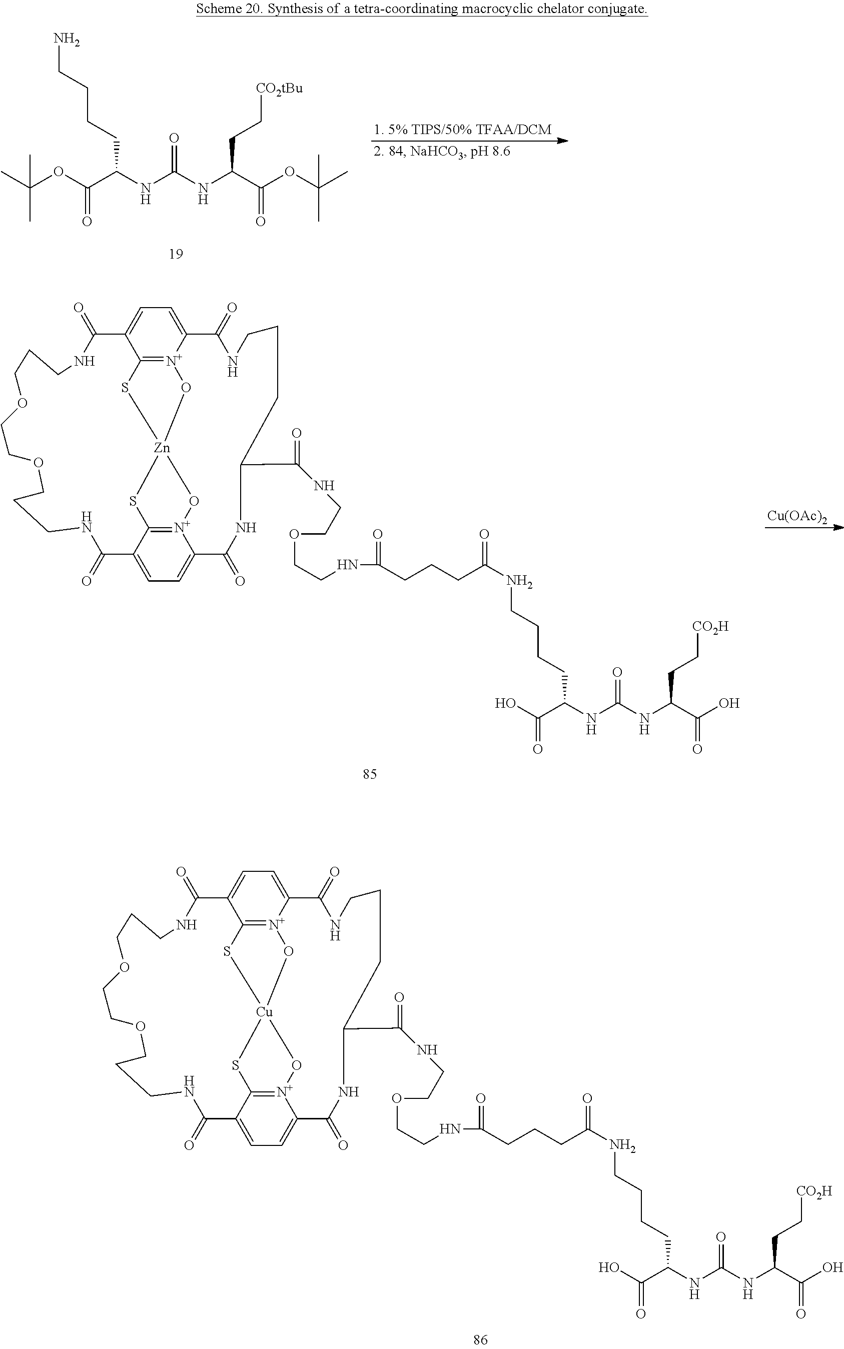

### Example 21. Synthesis of a Tetra-Coordinating Macrocyclic Chelator Conjugate

Compound 19 is treated with a mixture of triisopropylsilane, trifluoroacetic acid and dichloromethane overnight. Solvent is removed, and the residue is dissolve in an aqueous solution of sodium bicarbonate. A solution of compound 84 in dimethylformamide is added to the bicarbonate solution and allowed to mix for at least two hours to provide a solution of intermediate 85. A solution of copper acetate in dimethylformamide is added to convert compound 85 to compound 86. Compound 86 is isolated in purified form using reverse phase HPLC.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A chelating agent having a first chelating moiety having a structure according to Formula I: 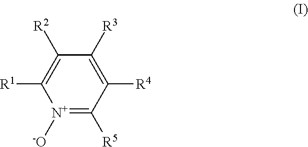
wherein
R¹, R², R³, and R⁴ are independently selected from a bond, H, substituted or unsubstituted alkyl, substituted or unsubstituted heteroalkyl, substituted or unsubstituted aryl, substituted or unsubstituted heteroaryl and substituted or unsubstituted heterocycloalkyl, halogen, CN, —CF₃, —C(O)R¹⁰, —SO₂NR¹⁰R¹¹, —NR¹⁰R¹¹, —OR¹⁰, —S(O)₂R¹⁰, —COOR¹⁰, —S(O)₂OR¹⁰, —OC(O)R¹⁰, —C(O)N R¹⁰R¹¹, —(CH₂)ₘC(O)NR¹⁰R¹¹, —O(CH₂)ₘC(O)NR¹⁰R¹¹, —NR¹⁰C(O)R¹¹, —NR¹⁰SO₂R¹¹, and —NO₂;
R⁵ is selected from SH, S—, OH and O—;
R¹⁰ and R¹¹ are independently selected from H, substituted or unsubstituted alkyl, substituted or unsubstituted heteroalkyl, substituted or unsubstituted aryl, substituted or unsubstituted heteroaryl and substituted or unsubstituted heterocycloalkyl; and R¹⁰ and R¹¹, together with the atoms to which they are attached, are optionally joined to form a 5-, 6- or 7-membered ring selected from substituted or unsubstituted cycloalkyl, substituted or unsubstituted heterocycloalkyl, substituted or unsubstituted aryl and substituted or unsubstituted heteroaryl; and
the index m is selected from 1, 2, 3, 4, 5, 6, 7, 8, 9, and 10.

2. The chelating agent according to claim 1, wherein the chelating agent is selected from linear chelating agents and macrocyclic chelating agents.

3. The chelating agent according to claim 1, wherein the chelating agent includes at least 2, 3, 4, 5, or 6 of the chelating moieties.

4. The chelating agent according to claim 1, wherein the chelating agent comprises a second chelating moiety and a linker linking the first chelating moiety and the second chelating moiety.

5. The chelating agent according to claim 1, having a structure according to Formula II: 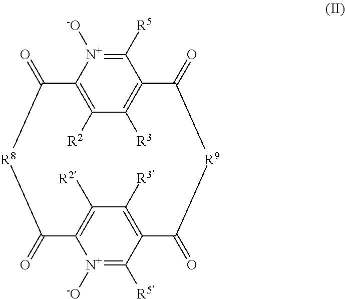
wherein
R^{1′}, and R^{2′} are independently selected from a bond, H, substituted or unsubstituted alkyl, substituted or unsubstituted heteroalkyl, halogen, CN, —CF₃, —C(O)R^{10′}, —SO₂NR^{10′}R^{11′}, —NR^{10′}R^{11′}, —OR^{10′}, —S(O)₂R^{10′}, —COOR^{10′}, —S(O)₂OR^{10′}, —OC(O)R^{10′}, —′C(O)NR^{10′}R^{11′}, —(CH₂)_{m′}C(O)NR^{10′}R^{11′}, —O(CH₂)ₘC(O)NR^{10′}R^{1′1}, —NR^{10′}C(O)R^{11′}, —NR^{10′}SO₂R^{11′}, and —NO₂;
R^{5′} is selected SH, S—, OH and O—;
R^{10′} and R^{11′} are independently selected from H, substituted or unsubstituted alkyl, substituted or unsubstituted heteroalkyl, substituted or unsubstituted aryl, substituted or unsubstituted heteroaryl and substituted or unsubstituted heterocycloalkyl; and R^{10′} and R^{11′}, together with the atoms to which they are attached, are optionally joined to form a 5-, 6- or 7-membered ring selected from substituted or unsubstituted cycloalkyl, substituted or unsubstituted heterocycloalkyl, substituted or unsubstituted aryl and substituted or unsubstituted heteroaryl;
the index m′ is selected from 1, 2, 3, 4, 5, 6, 7, 8, 9, and 10; and
R⁸ and R⁹ are independently members selected from substituted or unsubstituted alkyl, and substituted or unsubstituted heteroalkyl, substituted or unsubstituted aryl, substituted or unsubstituted heteroaryl and substituted or unsubstituted heterocycloalkyl.

6. The chelating agent according to claim 5, wherein one or more of R², R³, R^{5′}, R^{2′}, R^{5′}, R⁸ or R⁹ includes a linker to a reactive functional group or to a targeting moiety.

7. The chelating agent according to claim 1, having a structure according to Formula III: 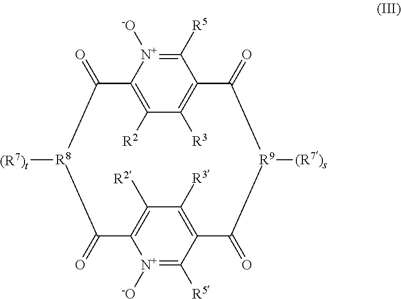
wherein
R⁷ and R^{7′} comprise 1, 2, 3, 4, 5, 6, 7, 8, 9, or 10 non-hydrogen atoms forming a linker linking the first chelating moiety of Formula III to a second chelating moiety of Formula III; and
the indices s and t are independently 0 or 1, with the proviso that at least one of s and t is 1 and s and t are not both 1.

8. The chelating agent according to claim 7, wherein a member selected from R⁷ and R^{7′} includes a reactive functional group or targeting moiety pendent from its terminus or an interior position.

9. The chelating agent according to claim 7, wherein a member selected from R⁷ and R^{7′} is substituted hydrocarbyl.

10. The chelating agent according to claim 7, wherein a member selected from R⁷ and R^{7′} includes one or more than one heteroatom.

11. The chelating agent according to claim 1, having a structure according to Formula (IV): 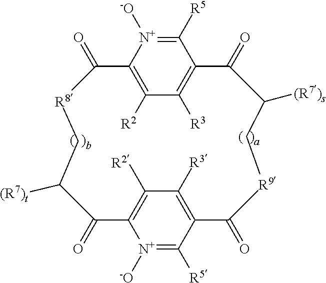
wherein
R^{8′} and R^{9′} are independently members selected from substituted or unsubstituted alkyl, and substituted or unsubstituted heteroalkyl , substituted or unsubstituted aryl, substituted or unsubstituted heteroaryl and substituted or unsubstituted heterocycloalkyl;
the indices s and t are 0 or 1, with the proviso that at least one of s and t is 1 and s and t are not both 1; and
the indices a and b are independently selected from 1, 2, 3, 4, 5, and 6.

12. The chelating agent according to any previous claim, having a structure according to Formula V:

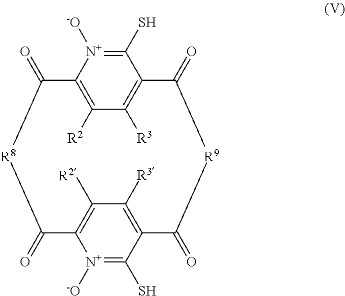

13. The chelating agent according to claim 1, having a structure according to Formula (VI): 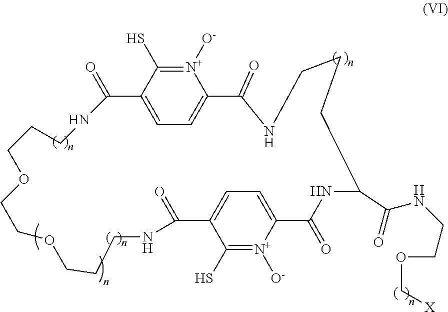
wherein
each index n is independently selected from the integers 0, 1, 2, 3, 4, 5, 6, 7, 8, 9, and 10; and
X is a reactive functional group or a targeting moiety.

14. The chelating agent according to claim 1, having a structure according to Formula IX: 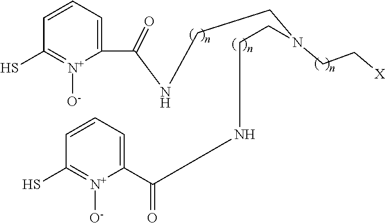
in which each index n is independently selected from 0, 1, 2, 3, 4, 5, 6, 7, 8, 9, and 10; and
X is a reactive functional group or a targeting moiety.

15. A pharmaceutical formulation comprising a chelating agent according to claim 1, and a member selected from pharmaceutically acceptable salt, pharmaceutically acceptable excipient, pharmaceutically acceptable carrier and a combination thereof.

16. The chelating agent, or the pharmaceutical formulation thereof, wherein the metal ion bound thereto is a soft Lewis acid metal ion.

17. The chelating agent, or the pharmaceutical formulation thereof of claim 1, wherein the metal ion bound thereto is a soft Lewis acid metal ion.

18. The chelating agent, or the pharmaceutical formulation thereof of claim 17, wherein the soft Lewis acid metal ion bound thereto is a member selected from Cu(II), Zn(II), Pb(II), Ga(III), ⁶⁴Cu(II), ⁶⁷Cu(II), ²¹²Pb(II), ⁶⁷Ga(III), ⁶⁸Ga(III), ⁶⁴Cu, ⁶⁷Cu, ²¹²Pb, ⁶⁷Ga, ⁶⁸Ga, Pt(II), Ni(II), and Cd(II).
